(19) United States
(12) Patent Application Publication
(10) Pub. No.: US 2024/0228561 A1
(43) Pub. Date: Jul. 11, 2024

(54) Methods Of Treating Non-Arteritic Anterior Ischemic Optic Neuropathy

(71) Applicant: Life Biosciences, Inc., Boston, MA (US)

(72) Inventors: Michel Wathier, Boston, MA (US); Jennifer Cermak, Boston, MA (US); Joan Mannick, Boston, MA (US)

(21) Appl. No.: 18/312,538

(22) Filed: May 4, 2023

Related U.S. Application Data
(60) Provisional application No. 63/499,864, filed on May 3, 2023.
(60) Provisional application No. 63/478,843, filed on Jan. 6, 2023.

(51) Int. Cl. C07K 14/47; A61K 31/65; A61P 25/02; C12N 15/86

(52) U.S. Cl. CPC C07K 14/4702; A61K 31/65; A61P 25/02; C12N 15/86

(57) ABSTRACT

Disclosed herein are methods for preventing or treating non-arteritic anterior ischemic optic neuropathy in a subject by administering to the subject a nucleic acid molecule comprising a nucleic acid sequence encoding OCT4, a nucleic acid sequence encoding SOX2, and a nucleic acid sequence encoding KLF4.

### CROSS-REFERENCE TO RELATED APPLICATIONS

This application claims the right of priority to U.S. Provisional Patent Application No. 63/478,843, filed Jan. 6, 2023, and to U.S. Provisional Patent Application No. 63/499,864, filed May 3, 2023, the contents of each of which are hereby expressly incorporated by reference in their entirety.

### INCORPORATION BY REFERENCE OF SEQUENCE LISTING

The instant application contains a Sequence Listing which has been submitted via EFS-Web. The content of the file named “061189-501001US_SequenceListing_ST26.xml”, which was created on May 3, 2023 and is 86,016 bytes in size, is hereby incorporated by reference in its entirety.

### TECHNICAL FIELD

The present patent application relates to methods of treating non-arteritic anterior ischemic optic neuropathy in a subject in need thereof using gene therapy.

### BACKGROUND

Ischemic optic neuropathy is the most common acute optic nerve disorder in patients over age 50 years. Ischemic optic neuropathy is generally categorized as anterior (affecting the optic disc) versus posterior (retrobulbar), and as arteritic versus nonarteritic. Anterior involvement is usual with both arteritic and nonarteritic ischemic optic neuropathy.

Nonarteritic anterior ischemic optic neuropathy (NAION) is the most common form of ischemic optic neuropathy. It is an idiopathic, ischemic insult of the optic nerve head characterized by acute, monocular, painless visual loss with optic disc swelling. According to the American Academy of Ophthalmology, NAION affects between 2.3 and 10.3 people per 100,000 individuals per year making it the most common cause of acute optic neuropathy in patients over the age of 50. There are approximately 6000 new cases per year. Men and women are nearly equally affected. [www_eyewiki_aao_org/Non-Arteritic_Anterior_Ischemic_Optic_Neuropathy_(NAION)].

There are no known treatments for NAION that are proven to be effective. There have been many clinical trials studying over a dozen different therapies, but none have convincingly improved the visual outcome in patients with NAION. The present disclosure addresses the need for such a treatment.

### SUMMARY

The present disclosure is directed to methods for preventing or treating non-arteritic anterior ischemic optic neuropathy in a subject by administering to the subject a nucleic acid molecule comprising a nucleic acid sequence encoding OCT4, a nucleic acid molecule comprising a nucleic acid sequence encoding SOX2, and a nucleic acid molecule comprising a nucleic acid sequence encoding KLF4. In some embodiments, the nucleic acid sequences encoding OCT4, SOX2, and KLF4 are on a single nucleic acid molecule. In certain aspects, an adeno-associated viral (AAV) vector comprises the nucleic acid molecule encoding OCT4, SOX2, and KLF4. In some embodiments, the nucleic acid molecule does not encode c-Myc and/or another transcription factors, such as Nanog.

In some embodiments, the methods further comprise administering to the subject a nucleic acid molecule comprising a nucleic acid sequence encoding a reverse tetracycline-controlled transactivator (rtTA). According to some embodiments of the methods, an AAV vector comprises the nucleic acid molecule encoding reverse tetracycline-controlled transactivator (rtTA). In some embodiments, the rtTA is rtTA3 or rtTA4. The AAV vector comprising the nucleic acid molecule encoding OCT4, SOX2, and KLF4 and the AAV vector comprising a nucleic acid molecule encoding reverse tetracycline-controlled transactivator (rtTA) may be present in a single AAV composition or as separate AAV compositions.

In some embodiments of the disclosed methods, the nucleic acid molecule comprising a nucleic acid sequence encoding OCT4, a nucleic acid sequence encoding SOX2, and a nucleic acid sequence encoding KLF4 is operably linked to an inducible promoter. In some embodiments, the inducible promoter comprises a tetracycline class antibiotic response element (TRE). In some embodiments, the tetracycline class antibiotic is doxycycline. In some embodiments, the inducible promoter is a TRE2 promoter.

In some embodiments of the disclosed methods, the nucleic acid molecule comprising a nucleic acid sequence encoding a rtTA is operably linked to a CMV promoter.

In some embodiments, the nucleic acid molecule comprising a nucleic acid sequence encoding OCT4, a nucleic acid sequence encoding SOX2, and a nucleic acid sequence encoding KLF4 is an adeno-associated viral (AAV) vector. In some embodiments, the AAV vector is serotype-2 (AAV2).

In some embodiments of the disclosed methods, the nucleic acid molecule comprising a nucleic acid sequence encoding OCT4, a nucleic acid sequence encoding SOX2, and a nucleic acid sequence encoding KLF4 does not comprise a nucleic acid sequence encoding c-Myc.

In some embodiments of the disclosed methods, the nucleic acid molecule comprising a nucleic acid sequence encoding OCT4, a nucleic acid sequence encoding SOX2, and a nucleic acid sequence encoding KLF4comprises a nucleic acid sequence encoding self-cleaving peptide. In some embodiments, the self-cleaving peptide is a 2A peptide.

In some embodiments of the disclosed methods, the nucleic acid molecule encoding OCT4, SOX2, and KLF4 is flanked by inverted terminal repeats (ITRs), and wherein the distance between the ITRs is 4.7 kb or less.

In some embodiments, the methods further comprise administering to the subject an inducing agent.

In some embodiments of the disclosed methods, the nucleic acid molecule encoding reverse tetracycline-controlled transactivator (rtTA) is an AAV vector that does not comprise the nucleic acid molecule encoding OCT4, SOX2, and KLF4. The nucleic acid molecule encoding reverse tetracycline-controlled transactivator (rtTA) may be an AAV vector comprising SEQ ID NO: 36 or SEQ ID NO: 37.

In some embodiments of the disclosed methods, the nucleic acid molecule encoding OCT4, SOX2, and KLF4 comprises nucleic acid elements in the following order: a) a first inverted terminal repeat sequence (ITR) sequence; b) a TRE promoter sequence; c) a nucleic acid sequence encoding OCT4; d) a nucleic acid sequence encoding P2A; e) a nucleic acid sequence encoding SOX2; f) a nucleic acid sequence encoding T2A; g) a nucleic acid sequence encoding KLF4; h) an SV-40-derived terminator sequence; and i) a second inverted terminal repeat (ITR) sequence.

In some embodiments of the disclosed methods, the nucleic acid sequence encoding OCT4 comprises SEQ ID NO: 1. In some embodiments, the nucleic acid sequence encoding SOX2 comprises SEQ ID NO: 3. In some embodiments, the KLF4 is human KLF4 protein. In some embodiments, the nucleic acid sequence encoding KLF4 comprises SEQ ID NO: 5. In some embodiments, the nucleic acid sequence encoding P2A comprises SEQ ID NO: 8. In some embodiments, the P2A comprises the amino acid sequence of SEQ ID NO: 9. In some embodiments, the T2A comprises the amino acid sequence of SEQ ID NO: 11.

In some embodiments, the nucleic acid sequence encoding T2A is

(SEQ ID NO: 10)
GAGGGCAGGGGAAGTCTTCTAACATGCGGGGACGTGGAGGAAAATCCCGG
CCCA.

In some embodiments, the TRE promoter sequence is SEQ ID NO: 7.

In some embodiments, the SV-40-derived terminator sequence is SEQ ID NO: 12.

In some embodiments, the first ITR sequence is SEQ ID NO: 16.

In some embodiments, the nucleic acid molecule encoding OCT4, SOX2, and KLF4 comprises SEQ ID NO: 13.

In some embodiments, the nucleic acid molecule encoding OCT4, SOX2, and KLF4 comprises SEQ ID NO:14.

In some embodiments, the nucleic acid molecule encoding OCT4, SOX2, and KLF4 and the nucleic acid molecule encoding rtTA are administered sequentially or simultaneously.

In some embodiments, the nucleic acid molecule encoding OCT4, SOX2, and KLF4 is administered intravitreally.

In some embodiments, the nucleic acid molecule encoding the rtTA is administered intravitreally.

In some embodiments, the nucleic acid molecule encoding OCT4, SOX2, and KLF4 and the nucleic acid molecule encoding rtTA are administered at a ratio of about 1:1.

In some embodiments, the AAV vector comprising the nucleic acid molecule encoding OCT4, SOX2, and KLF4 and the AAV vector comprising the nucleic acid molecule encoding rtTA are administered at a ratio of about 1:1 (vg:vg).

In some embodiments, the nucleic acid molecule encoding OCT4, SOX2, and KLF4 is AAV2-TRE-OSK vector and the nucleic acid molecule encoding rtTA is AAV2-CMV-rtTA3 or AAV2-CMV-rtTA4.

In some embodiments, the effective amount of the AAV2-TRE-OSK vector is in the range of from about 1×10⁹ vg/eye to about 1×10¹⁴ vg/eye.

In some embodiments, the effective amount of the AAV2-CMV-rtTA3 vector is in the range of from about 1×10⁹ vg/eye to about 1×10¹⁴ vg/eye.

In some embodiments, the effective amount of the AAV2-CMV-rtTA4 vector is in the range of from about 1×10⁹ vg/eye to about 1×10¹⁴ vg/eye.

In some embodiments, the nucleic acid molecule encoding OCT4, SOX2, and KLF4 is administered to the subject by oculus sinister (OS) injection, by oculus dexter (OS) injection, or by oculus uterque (OU) injection.

In some embodiments, the nucleic acid molecule encoding rtTA is administered to the subject by oculus sinister (OS) injection, by oculus dexter (OS) injection, or by oculus uterque (OU) injection.

In some embodiments, the methods further comprise administering to the subject an effective amount of an antibiotic. In some embodiments, the antibiotic is administered at least one day prior to administering the nucleic acid molecule encoding rtTA. In some embodiments, the antibiotic is administered when the nucleic acid molecule encoding rtTA is administered. In some embodiments, the antibiotic is administered at least one day following administration of the nucleic acid molecule encoding rtTA.

In certain aspects, provided herein are methods for recombinant preparation of an AAV, the method comprising introducing a vector into a cell under conditions whereby the AAV is produced, wherein the vector comprises one or more nucleic acid sequences encoding a) OCT4, SOX2, and KLF4. In some embodiments, the cell comprises a population of HEK293T cells.

Further provided herein are methods of generating an AAV comprising modifying a cell to express one or more plasmids comprising: one or more AAV2 Rep-Cap plasmids, one or more helper plasmids, and one or more transfer plasmids, wherein the one or more transfer plasmids comprise one or more nucleic acids encoding OCT4, SOX2, and KLF4. In some embodiments, the cell comprises a population of HEK293T cells.

In certain aspects, provided herein are methods for preventing or treating non-arteritic anterior ischemic optic neuropathy (NAION) in a subject in need thereof, the method comprising administering to the subject a pharmaceutically effective amount of a composition comprising an expression vector comprising a polynucleotide encoding OCT4. SOX2, and KLF4, but not c-Myc. In some embodiments, the polynucleotide further does not encode one or more transcription factors, such as Nanog.

In certain aspects, provided herein are methods for preventing or treating non-arteritic anterior ischemic optic neuropathy (NAION) in a subject in need thereof, the method comprising administering to the subject a pharmaceutically effective amount of a composition comprising an expression vector encoding three transcription factors, wherein the transcription factors consist of OCT4, SOX2, and KLF4.

In certain aspects, provided herein are methods for preventing or treating non-arteritic anterior ischemic optic neuropathy (NAION) in a subject in need thereof, the method comprising administering to the subject a composition comprising an expression vector comprising a polynucleotide encoding four or more transcription factors, wherein the transcription factors comprise OCT4, SOX2, and KLF4, but not c-Myc. In some embodiments, the polynucleotide further does not encode one or more transcription factors, such as Nanog.

In some embodiments, the composition does not reprogram the cell, tissue, or organ to a pluripotent state in the subject. In some embodiments, the composition rejuvenates at least one cell, tissue, or organ in the subject. In some embodiments, the composition does not induce c-Myc expression in the subject. In some embodiments, the composition does not induce expression of one or more transcription factors (e.g., Nanog) in the subject. In some embodiments, the composition does not induce expression of at least one stem cell marker in the subject. Such at least one stem cell marker may comprise Esrrb, Nanog, Lin28, TRA-1-60/TRA-1-81/TRA-2-54, SSEA1, SSEA4, or any combination thereof. In some embodiments, the composition induces expression of RBPMS, Bm3a, or a combination thereof in the subject. In some embodiments, rejuvenating at least one cell, tissue, or organ comprises increasing repair and/or regeneration in the cell, tissue, or organ. In some embodiments, rejuvenating at least one cell, tissue, or organ comprises restoring epigenetic information in the subject. In some embodiments, rejuvenating at least one cell, tissue, or organ comprises restoring epigenetic information lost due to aging, injury, disease, or any combination thereof in the cell, tissue, or organ. In some embodiments, rejuvenating at least one cell, tissue, or organ comprises reestablishing the epigenetic status of the cell, tissue, or organ to an epigenetic status closer to fertilization or final differentiation. In some embodiments, rejuvenating at least one cell, tissue, or organ comprises increasing the number of healthy axons in the subject. In some embodiments, rejuvenating at least one cell, tissue, or organ comprises preventing damages to healthy axons in the subject.

In some embodiments, the polynucleotide comprises DNA, RNA, or a combination thereof. In some embodiments, the DNA comprises a plasmid DNA. In some embodiments, the RNA comprises an mRNA. In some embodiments, the polynucleotide comprises an inducible promoter, such as a TRE3G promotor, a TRE2 promoter, a P tight promoter, and a tetracycline response element (TRE).

In some embodiments, the methods described herein further comprises administering to the subject an inducing agent to induce expression of OCT4, SOX2, and KLF4 in the subject. In some embodiments, the inducing agent comprises a tetracycline class antibiotic, such as doxycycline. In some embodiments, the inducing agent comprises a reverse tetracycline-controlled transactivator (rtTA) or a polynucleotide encoding the rtTA. In some embodiments, the polynucleotide encoding the rtTA is in an expression vector.

In some embodiments, the composition and the inducing agent is administered sequentially or simultaneously. In some embodiments, the composition is administered prior to administering the inducing agent. In some embodiments, the composition is administered after administering the inducing agent. In some embodiments, the composition is administered simultaneously with the inducing agent.

In some embodiments, the composition and the inducing agent is administered at a ratio of about 100:1, 50:1, 40:1, 30:1, 25:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:25, 1:30, 1:40, 1:50 or 1:100. In some embodiments, the composition and the inducing agent is administered at a ratio of more than about 100:1. In some embodiments, the composition and the inducing agent is administered at a ratio of less about 1:100. In some embodiments, the composition and the inducing agent is administered at a ratio of about 1:1.

In some embodiments, the polynucleotide comprises a self-cleaving peptide, such as a 2A peptide.

In some embodiments, the polynucleotide comprises inverted terminal repeats (ITRs).

In some embodiments, the expression vector is a viral expression vector selected from a lentivirus, a retrovirus, an adenovirus, alphavirus, vaccinia virus, and an adeno-associated virus (AAV) vector. In some embodiments, the AAV vector is serotype-2 (AAV2).

In some embodiments, the polynucleotide comprises nucleic acid elements in the following order:
a. a first inverted terminal repeat sequence (ITR) sequence;
b. a TRE3G promoter sequence;
c. an OCT4 sequence;
d. a P2A cleavage sequence;
e. a SOX2 sequence;
f. a T2A cleavage sequence;
g. a KLF4 sequence;
h. an SV-40-derived terminator sequence; and
i. a second inverted terminal repeat (ITR) sequence.

In some embodiments,
i) OCT4 comprises an amino acid sequence having at least 70%, 75%, 80%, 85%, 90%, 91%, 92%, 93%, 94%, 95%, 96%, 97%, 98%, 99%, or more, identity to SEQ ID NO: 2;
ii) SOX2 comprises an amino acid sequence having at least 70%, 75%, 80%, 85%, 90%, 91%, 92%, 93%, 94%, 95%, 96%, 97%, 98%, 99%, or more, identity to SEQ ID NO: 4; and/or
iii) KLF4 comprises an amino acid sequence having at least 70%, 75%, 80%, 85%, 90%, 91%, 92%, 93%, 94%, 95%, 96%, 97%, 98%, 99%, or more, identity to SEQ ID NO: 6.

In some embodiments,
i) OCT4 comprises an amino acid sequence having at least 90% identity to SEQ ID NO: 2;
ii) SOX2 comprises an amino acid sequence having at least 90% identity to SEQ ID NO: 4; and/or
iii) KLF4 comprises an amino acid sequence having at least 90% identity to SEQ ID NO: 6.

In some embodiments,
i) OCT4 comprises an amino acid sequence having at least 95% identity to SEQ ID NO: 2;
ii) SOX2 comprises an amino acid sequence having at least 95% identity to SEQ ID NO: 4; and/or
iii) KLF4 comprises an amino acid sequence having at least 95% identity to SEQ ID NO: 6.

In some embodiments,
i) OCT4 comprises the amino acid sequence of SEQ ID NO: 2;
ii) SOX2 comprises the amino acid sequence of SEQ ID NO: 4; and/or
iii) KLF4 comprises the amino acid sequence of SEQ ID NO: 6.

In some embodiments,
i) the polynucleotide comprises a nucleic acid sequence having at least 70%, 75%, 80%, 85%, 90%, 91%, 92%, 93%, 94%, 95%, 96%, 97%, 98%, 99%, or more, identity to SEQ ID NO: 1;
ii) the polynucleotide comprises a nucleic acid sequence having at least 70%, 75%, 80%, 85%, 90%, 91%, 92%, 93%, 94%, 95%, 96%, 97%, 98%, 99%, or more, identity to SEQ ID NO: 3; and/or
iii) the polynucleotide comprises a nucleic acid sequence having at least 70%, 75%, 80%, 85%, 90%, 91%, 92%, 93%, 94%, 95%, 96%, 97%, 98%, 99%, or more, identity to SEQ ID NO: 5.

In some embodiments,
i) the polynucleotide comprises a nucleic acid sequence having at least 75% identity to SEQ ID NO: 1;
ii) the polynucleotide comprises a nucleic acid sequence having at least 75% identity to SEQ ID NO: 3; and/or
iii) the polynucleotide comprises a nucleic acid sequence having at least 75% identity to SEQ ID NO: 5.

In some embodiments,
i) the polynucleotide comprises a nucleic acid sequence having at least 90% identity to SEQ ID NO: 1;
ii) the polynucleotide comprises a nucleic acid sequence having at least 90% identity to SEQ ID NO: 3; and/or
iii) the polynucleotide comprises a nucleic acid sequence having at least 90% identity to SEQ ID NO: 5.

In some embodiments,
i) the polynucleotide comprises a nucleic acid sequence of SEQ ID NO: 1;
ii) the polynucleotide comprises a nucleic acid sequence of SEQ ID NO: 3; and/or
iii) the polynucleotide comprises a nucleic acid sequence of SEQ ID NO: 5.

In some embodiments, the composition is administered systematically. In some embodiments, the composition is administered locally to a tissue or organ. In some embodiments, the composition is administered intravitreally. In some embodiments, the composition is administered to the subject by oculus sinister (OS) injection, by oculus dexter (OD) injection, or by oculus uterque (OU) injection.

In some embodiments, administering the composition improves retinal ganglion cell (RGC) function and/or restores visual function in the subject.

In some embodiments, the preventing or treating NAION is measurable by electroretinogram (pERG). In some embodiments, the preventing or treating NAION is measured by electroretinogram (pERG).

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments thereof; however, the methods are not limited to the specific embodiments disclosed. In the drawings:

FIGS. 1A and 1B depict results from pattern electroretinogram (pERG) measurements showing induction of nonarteritic anterior ischemic optic neuropathy (NAION) results in decreased pERG signal. The pERG uses contrast reversing pattern stimuli (checkerboards) to assess macular retinal ganglion cell (RGC) activity. Changes in the pERG waveform are indicative of RGCs dysfunctions. FIG. 1A shows pERG data from both left (OS) and right (OD) eyes from experiments where the right eye received no treatment and the left eye was treated with vehicle (phosphate-buffered saline (PBS)) and subjected to induction of the NAION injury. Results show laser-induced decrease in peak amplitude (the left panel) and the absolute amplitude (the right panel) that is similar to that seen in NAION and is indicative of retinal ganglion cell (RGC) dysfunction. FIG. 1B shows pERG data from both left (OS) and right (OD) eyes from experiments where the right eye received no treatment (contralateral eye) and the left eye was injured with laser and administered the vehicle 1 day post-injury. Results show significant decreases in p50 amplitude (the left panel) and absolute amplitude from p50 to n95 (the right panel) indicating impairment of retinal ganglion cell (RGC) function.

FIG. 2 is an illustrative vector map of TRE3G-OSK-SV40 pA, an AAV2 vector encoding OSK (SEQ ID NO: 15).

FIG. 3 depicts an illustrative vector map of pAAV2-CMV-rtTA3(VP16) (SEQ ID NO: 21). This vector is a non-limiting example of a vector encoding rtTA.

FIG. 4 depicts an illustrative vector map of pAAV2-CMV-rtTA4 (SEQ ID NO: 28). This vector is a non-limiting example of a vector encoding rtTA.

FIG. 5 depicts a schematic showing a non-limiting example of a Tet-ON system to express OCT4, SOX2, and KLF4 (OSK) in the presence of a tetracycline.

FIG. 6 depicts a schematic comparing the p50 peak amplitude (the left panel) or the absolute amplitude from p50 to n95 (the right panel) when treated with vehicle (the left bar at each time point) or OSK (controlled by Dox; as Tet-on system; the right bar at each time point) before laser treatment (i.e., prevention study). pERG signals were measured at different days and compared under different treatment.

FIG. 7 depicts a schematic comparing the p50 peak amplitude (the left panel) or the absolute amplitude from p50 to n95 (the right panel) when treated with vehicle (the left bar at each time point) or OSK (controlled by Dox; as Tet-on system; the right bar at each time point) after laser treatment (i.e., rescue study). pERG signals were measured at different days and compared under different treatment.

FIG. 8 depicts optic nerve myelin-specific axon stain microscopy results, which are used for axon counting by using AxonNet, for control (“no injury”; no laser treatment), or laser treatment followed by vehicle or OSK treatment.

FIG. 9 depicts illustrative axon-counting results, from experiments as described in FIG. 8. Median values (middle bars) and interquartile ranges (top and bottom bars) are shown for each treatment/control.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Throughout this text, the description refers to compositions and methods of using the compositions. Where the disclosure describes or claims a feature or embodiment associated with a composition, such a feature or embodiment is equally applicable to the methods of using the composition. Likewise, where the disclosure describes or claims a feature or embodiment associated with a method of using a composition, such a feature or embodiment is equally applicable to the composition.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Unless otherwise defined, all technical and scientific terms used herein have the same meaning as commonly understood by one of ordinary skill in the art. In case of conflict, the present document, including definitions, will control. Exemplary methods and materials are described below, although methods and materials similar or equivalent to those described herein can be used in practice or testing of the disclosed methods. All publications, patent applications, patents and other references mentioned herein are incorporated by reference in their entirety. The materials, methods, and examples disclosed herein are illustrative only and not intended to be limiting. The terminology used herein is for the purpose of describing particular embodiments only and is not intended to be limiting.

The terms “comprise(s),” “include(s),” “having.” “has.” “can,” “contain(s),” and variants thereof, as used herein, are intended to be open-ended transitional phrases. terms, or words that do not preclude the possibility of additional acts or structures. The term “comprising” is intended to include examples encompassed by the terms “consisting essentially of” and “consisting of”; similarly, the term “consisting essentially of” is intended to include examples encompassed by the term “consisting of.” The present disclosure also contemplates other embodiments “comprising.” “consisting of,” and “consisting essentially of” the embodiments or elements presented herein, whether explicitly set forth or not.

The singular forms “a.” “and” and “the” include plural references unless the context clearly dictates otherwise.

For recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

Some of the quantitative expressions given herein are not qualified with the term “about.” It is understood that, whether the term “about” is used explicitly or not, every quantity given is intended to refer to the actual given value, and it is also meant to refer to the approximation to such given value that would reasonably be inferred based on the ordinary skill in the art, including approximations due to the experimental and/or measurement conditions for such value.

“AAV” or “adeno-associated virus” is a nonenveloped virus that is capable of carrying and delivering nucleic acids (e.g., engineered nucleic acids encoding OCT4; KLF4; SOX2; or any combination thereof) and belongs to the genus Dependoparvovirus. In some instances, an AAV is capable of delivering a nucleic acid encoding an inducing agent. In general, AAV does not integrate into the genome. The tissue-specific targeting capabilities of AAV is often determined by the AAV capsid serotype (see, e.g., Table 1 below for examples of AAV serotypes and their utility in tissue-specific delivery). Non-limiting serotypes of AAV include AAV1, AAV2, AAV3, AAV4, AAV5, AAV6, AAV7, AAV8, AAV9. AAV 10. AAV11, and variants thereof. In certain embodiments, the AAV serotype is a variant of AAV9 (e.g., AAV PHP.b).

TABLE 1
Non-limiting Examples of AAV Serotypes and their Use in Specific Tissues

| | Relevant Tissue | | | | | | | | |
|---|---|---|---|---|---|---|---|---|---|
| | | | | | | Central | | | Immune |
| | | | | | | Nervous | | | System |
| | | | Muscle | | Central | System | | | (T-cells, |
| | | | (e.g., | | Nervous | (Blood- | | | B-cells and |
| AAV | | | Skeletal | | System | brain | | | Dendritic |
| Serotype | Liver | Heart | Muscle) | Eye | (CNS) | barrier) | Pancreas | Lung | Cells) |
| AAV1 | | X | X | | X | | | | |
| AAV2 | X | | X | X | X | | | | |
| AAV3 | X | | X | X | | | | X | |
| AAV4 | | | X | X | | | | | |
| AAV5 | | | X | X | | | X | X | |
| AAV6 | | X | X | | | | | X | X |
| (e.g., | | | | | | | | | |
| AAV6.2) | | | | | | | | | |
| AAV7 | X | | X | | | | | | |
| AAV8 | X | | X | | X | | X | | |
| AAV9 | X | X | X | X | X | X | X | X | |
| AAV10 | X | X | X | X | X | X | X | X | |
| (e.g., | | | | | | | | | |
| AAVrh10) | | | | | | | | | |
| AAVDJ | X | | X | | X | | | | |
| AAVPHP.B | | | | | X | X | | | |

A “recombinant virus” is a virus (e.g., lentivirus, adenovirus, retrovirus, herpes virus, alphavirus, vaccinia virus or adeno-associated virus (AAV)) that has been isolated from its natural environment (e.g., from a host cell, tissue, or a subject) or is artificially produced.

The term “AAV vector” as used herein is a nucleic acid that comprises AAV inverted terminal repeats (ITRs) flanking an expression cassette (e.g., an expression cassette comprising a nucleic acid encoding OCT4, KLF4, and SOX2, each alone or in combination, or an expression cassette encoding rtTA or (TA). An AAV vector may further comprise a promoter sequence.

The terms “administer,” “administering.” or “administration,” as used herein refers to introduction of any of the compositions described herein; any of the nucleic acids capable of inducing OCT4, KLF4, and/or SOX2 expression; any of the nucleic acids capable of inducing expression of one or more transcription factors selected from the group consisting of OCT4, KLF4, SOX2, and any combinations thereof; any of the engineered proteins described herein; any of the chemical agents activating (e.g., inducing expression of) OCT4, KLF4, and/or SOX2; any of the chemical agents activating (e.g., inducing expression of) one or more transcription factors selected from OCT4, KLF4, SOX2, and any combinations thereof; any of the antibodies activating (e.g., inducing expression of) OCT4, KLF4, and/or SOX2, and any combinations thereof; and/or any of the recombinant viruses (e.g., lentivirus, adenovirus, alphavirus, vaccinia virus, retrovirus, herpes virus, or AAV) described herein, alone, or in combination to any cell, tissue, organ, and/or subject. In some embodiments, a nucleic acid encoding an inducing agent, an engineered protein encoding an inducing agent, a chemical agent capable of modulating (e.g., activating or inhibiting) the activity of an inducing agent, and/or a recombinant virus encoding an inducing agent is also administered to the cell, tissue, organ and/or subject. Any of the compositions described herein, comprising any of the nucleic acids capable of inducing expression of one or more transcription factors selected from OCT4, KLF4, SOX2, and any combinations thereof; any of the chemical agents activating (e.g., inducing expression of, e.g., tetracyline) OCT4, KLF4, and/or SOX2; any of the engineered proteins encoding OCT4, SOX2, KLF4, or any combinations thereof; any of the chemical agents activating (e.g., inducing expression of, e.g., tetracyline) OCT4, KLF4, SOX2, or any combination thereof; any of the antibodies activating (e.g., inducing expression of) OCT4, KLF4, and/or SOX2; and/or any of the recombinant viruses (e.g., lentivirus, adenovirus, alphavirus, vaccinia virus, retrovirus, herpes virus, or AAV) described herein, alone, or in combination may be administered intravitreally, intraocularly, subconjuctivally, or subretinally. In other aspects, administration can be intravenously, intradermally, intraarterially, intralesionally, intratumorally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, in lipid compositions (e.g., liposomes), or by other method or any combination of the forgoing as would be known to one of ordinary skill in the art (see, for example, Remington's Pharmaceutical Sciences (1990), incorporated herein by reference). In some embodiments, a composition comprising a nucleic acid encoding an inducing agent, an engineered protein encoding an inducing agent, a chemical agent capable of modulating (e.g., activating or inhibiting) the activity of an inducing agent, and/or a recombinant virus encoding an inducing agent is also administered to the cell, tissue, organ and/or subject using any suitable method such as intravitreally, intraocularly. subconjuctivally, or subretinally.

As used herein, the term “cell” is meant not only to include an individual cell but refers also to the particular tissue or organ from which it originates.

The term “gene expression” refers to the degree to which certain genes or all genes in a cell or tissue are transcribed into RNA. In some instances, the RNA is translated by the cell into a protein. The epigenome dictates gene expression patterns.

The terms “condition,” “disease,” and “disorder” are used interchangeably. As used herein, an “ocular disease” or “eye disease” is a disease or condition of the eye. An example of an ocular disease is Non-arteritic anterior ischemic optic neuropathy.

Any suitable method may be used to measure ocular function. Non-limiting examples include visual acuity tests, pattern electroretinograms (pERGs), and pathology.

“Cellular causes of aging” as used herein include loss or modification of epigenetic information.

The terms “c-Myc” or “Myc” refer to a nuclear phosphoprotein that has been implicated in cell cycle progression. c-Myc is capable of forming a heterodimer with the transcription factor MAX and the heterodimer is capable of binding to an E box consequence sequence on nucleic acids (e.g., engineered nucleic acids) to regulate transcription of target genes. In certain embodiments, a nucleotide sequence encoding c-Myc comprises a sequence that is at least 70% (e.g., at least 75%, 80%, 85%, 90%, 95%, 98%, 99%, or 100%) identical to a sequence as described in the NCBI RefSeq database under accession number NM_001354870.1 or NM_002467.5. In certain embodiments, an amino acid sequence encoding c-Myc comprises a sequence that is at least 70% (e.g., at least 75%, 80%, 85%, 90%, 95%, 98%, 99%, or 100%) identical to NP 002458.2 or NP 001341799.1. In certain embodiments, the methods comprise inducing expression of OCT4; KLF4; SOX2; or any combination thereof in the absence of inducing c-Myc expression or in the absence of activating c-Myc. Absence of inducing c-Myc expression may refer to absence of substantial induction of c-Myc expression over endogenous levels of c-Myc expression in a cell, tissue, subject, or any combination thereof. Absence of substantial induction of c-Myc expression as compared to endogenous levels of c-Myc expression in a cell, tissue, subject, or any combination thereof, may refer to increasing c-Myc expression by less than 70%, less than 60%, less than 50%, less than 40%, less than 30%, less than 20%, less than 10%, or any values in between as compared to endogenous levels of c-Myc expression in the cell, tissue, subject, or any combination thereof. Absence of activating c-Myc expression may refer to absence of substantial activation of c-Myc (e.g., activity) over endogenous c-Myc activity in a cell, tissue, subject, or any combination thereof. Absence of substantial induction of c-Myc activity as compared to endogenous c-Myc activity in a cell, tissue, subject, or any combination thereof, may refer to increasing c-Myc activity by less than 70%, less than 60%. less than 50%, less than 40%, less than 30%, less than 20%, less than 10%, or any values in between as compared to endogenous c-Myc activity in the cell, tissue, subject, or any combination thereof.

The terms “effective amount” and “therapeutically effective amount,” as used herein, refer to the amount of a compound or composition, that, when administered to a subject, is effective to at least partially treat a condition from which the subject is suffering.

As used herein, a protein that is “functional” or “active” is one that retains its biological activity (e.g., capable of acting as a transcription factor or as an inducing agent). Conversely, a protein that is not functional or is inactive is one that is not capable of performing one or more of its wild-type functions.

The term “gene” refers to a nucleic acid fragment that expresses a protein, including regulatory sequences preceding (5 ‘ non-coding sequences) and following (3’ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

“Homolog” or “homologous” refers to sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity). Homologous sequences include but are not limited to paralogous or orthologous sequences. Paralogous sequences arise from duplication of a gene within a genome of a species, while orthologous sequences diverge after a speciation event. A functional homolog retains one or more biological activities of a wild-type protein. In certain embodiments, a functional homolog of OCT4, KLF4, or SOX2 retains at least 10%, at least 20%, at least 30%, at least 40%, at least 50%, at least 60%, at least 70%, at least 80%, at least 90%, or at least 100% of the biological activity (e.g., transcription factor activity) of a wild-type counterpart.

“KLF4” may also be referred to as Kruppel-like factor 4, EZF, or GKLF and is a zinc-finger transcription factor. KLF4 has been implicated in regulation of differentiation and proliferation and is capable of interacting with co-activators, including members of the p300-CBP coactivator family. A KLF4 transcription factor, homolog (e.g., functional homolog), or variant thereof, as used herein, may be derived from any species, including humans. In certain embodiments, the nucleic acid encoding human KLF4 comprises a sequence that is at least 70% (e.g., at least 75%, 80%, 85%, 90%, 95%, 98%, 99%, or 100%) identical to a nucleic acid described in the NCBI RefSeq database under accession number NM_004235.5 or NM_001314052.1. Non-limiting examples of KLF4 variants include Kruppel-like factor 4 transcript variant 1 and Kruppel-like factor 4 transcript variant 2. In certain embodiments, KLF4 is encoded by a nucleic acid molecule comprising a nucleic acid sequence that is at least 70% (e.g., at least 75%, 80%, 85%, 90%, 95%, 98%, 99%, or 100%) identical to SEQ ID NO: 5. SEQ ID NO: 5 is a non-limiting example of a nucleotide sequence encoding human KLF4. In certain embodiments, KLF4 comprises an amino acid sequence that is at least 70% (e.g., at least 75%, 80%, 85%, 90%, 95%, 98%, 99%, or 100%) identical to NP_001300981.1 or NP_004226.3. In certain embodiments, KLF4 comprises an amino acid sequence that is at least 70% (e.g., at least 75%, 80%, 85%, 90%, 95%, 98%, 99%, or 100%) identical to SEQ ID NO: 6. SEQ ID NO: 6 is a non-limiting example of an amino acid sequence of human KLF4.

“Inverted terminal repeats” or “ITRs” are nucleic acid sequences that are reverse complements of one another. In general, in an AAV vector, ITRs are found on either side of a cassette (e.g., an expression cassette comprising a nucleic acid encoding OCT4; KLF4; SOX2; or any combination thereof). For example, the ITRs flanking the OSK cassette may comprise SEQ ID NOs: 16 and 32. Similarly, in some instances, the AAV2-CMV-rtTA3 vector disclosed herein can include ITRs comprising SEQ ID NOs: 22 and 33, and the AAV2-CMV-rtTA4 vector disclosed herein can include ITRs comprising SEQ ID NOs: 29 and 34. In some instances, the cassette encodes an inducing agent. AAV ITRs include ITRs from AAV1, AAV2, AAV3, AAV4, AAV5, AAV6, AAV7, AAV8, AAV9, AAV 10, AAV11, and AAV variants thereof.

The terms “nucleic acid,” “polynucleotide”, “nucleotide sequence”, “nucleic acid molecule”, “nucleic acid sequence”, and “oligonucleotide” refer to a series of nucleotide bases (also called “nucleotides”) in DNA and RNA, and mean any chain of two or more nucleotides. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids.

The nucleic acids described herein may be synthesized by standard methods known in the art, e.g., by use of an automated DNA synthesizer (such as those that are commercially available from Biosearch, Applied Biosystems, etc.). Such DNA sequences may be incorporated into a wide variety of vectors that incorporate suitable RNA polymerase promoters such as the T7 or SP6 polymerase promoters. A vector can be introduced in vivo such that it is taken up by a cell and directs the transcription of the nucleic acid molecule. Such a vector can remain episomal or become chromosomally integrated, as long as it can be transcribed. Such vectors can be constructed by recombinant DNA technology methods standard in the art. Vectors can be plasmid, viral, or others known in the art, used for replication and expression in mammalian cells. Expression of the sequence can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be administered to the subject.

The nucleic acid molecules may include natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5″-and 3 ″-non-coding regions, and the like. A “recombinant nucleic acid molecule” or “engineered nucleic acid molecule” is a nucleic acid molecule that has undergone a molecular biological manipulation, i.e., non-naturally occurring nucleic acid molecule or genetically engineered nucleic acid molecule. Furthermore, the terms “recombinant DNA molecule” or “engineered nucleic acid” refer to a nucleic acid sequence which is not naturally occurring, or can be made by the artificial combination of two otherwise separated segments of nucleic acid sequence, i.e., by ligating together pieces of DNA that are not normally contiguous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al. Molecular Cloning, second edition, Cold Spring Harbor Laboratory, Plainview, N.Y.; (1989), or Ausubel el al, Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature. Restriction enzyme recognition sites are often the target of such artificial manipulations, but other site-specific targets, e.g., promoters, DNA replication sites, regulation sequences, control sequences, open reading frames, or other useful features may be incorporated by design.

“OCT4” may also be referred to as Octamer-binding transcription factor 4, OCT3, OCT3/4, POU5F1, or POU class 5 homeobox 1 and is a transcription factor that has been implicated in embryonic development and determination of cell fate. Similar to other OCT transcription factors, OCT4 is characterized by a bipartite DNA binding domain called a POU domain. An OCT4 transcription factor, homolog, or variant thereof, as used herein, may be derived from any species, including humans. In certain embodiments, the nucleic acid encoding human OCT4 is at least 70% (e.g., at least 75%, 80%, 85%, 90%, 95%, 98%, 99%, or 100%) identical to a nucleic acid described in the NCBI RefSeq under accession number NM_002701, NM_203289, NM_001 173531, NM_001285986, or NM_001285987. In certain embodiments, the nucleic acid molecule encoding a human OCT4 comprises a nucleic acid sequence that is at least 70% (e.g., at least 75%, 80%, 85%, 90%, 95%, 98%, 99%, or 100%) identical to a nucleic acid sequence provided as SEQ ID NO: 1. SEQ ID NO: 1 is a non-limiting example of a nucleotide sequence encoding human OCT4. Non-limiting examples of OCT4 variants encompassed herein include POU5F1, transcript variant 1, POU5F1, transcript variant 2, POU5F1, transcript variant 3, POU5F1, transcript variant 4, and POU5F1 transcript variant 5. In certain embodiments, the nucleic acid molecule encodes an OCT4 comprising an amino acid sequence at least 70% (e.g., at least 75%, 80%, 85%, 90%, 95%, 98%, 99%, or 100%) identical to an amino acid sequence described in the NCBI RefSeq under accession number NP 001167002.1, NP_001272915.1, NP_001272916.1, NPJ302692.2, or NP_976034.4. In certain embodiments, the nucleic acid molecule encodes an OCT4 comprising an amino acid sequence that is at least 70% (e.g., at least 75%, 80%, 85%, 90%, 95%, 98%, 99%, or 100%) identical to SEQ ID NO: 2. SEQ ID NO: 2 is a non-limiting example of an amino acid sequence of human OCT4. Other OCT4 transcription factors (e.g., from other species) are known and nucleic acids encoding OCT4 transcription factors can be found in publicly available databases, including GenBank.

The term “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific, or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation of that sequence, expression of that sequence, or a combination thereof.

A promoter may promote ubiquitous expression or tissue-specific expression of an operably linked nucleic acid sequence from any species, including humans. In some embodiments, the promoter is a eukaryotic promoter. Non limiting examples of eukaryotic promoters include TDH3, PGK1, PKC1, TDH2, PYK1, TPI1, AT1, CMV, EF1 alpha, SV40, PGK1 (human or mouse), Ubc, human beta actin, CAG, TRE, UAS, Ac5, Polyhedrin, CaMKIIa, GAL1, GAL10, TEF1, GDS, ADH1, CaMV35S, Ubi, Hl, and U6, as would be known to one of ordinary skill in the art (see, e.g., Addgene website: blog. addgene.org/plasmids-101-the-promoter-region).

Non-limiting examples of ubiquitous promoters include tetracycline-responsive promoters (under the relevant conditions), CMV (e.g., SEQ ID NO: 17), EF1 alpha, a SV40 promoter, PGK1, Ubc, CAG, human beta actin gene promoter, a RSV promoter, an EFS promoter, and a promoter comprising an upstream activating sequence (UAS). In certain embodiments, the promoter is a mammalian promoter.

Non-limiting examples of tissue-specific promoters include eye-specific promoters. Non-limiting examples of eye-specific promoters include human GRK1 (rhodopsin kinase) promoter, human CRX (cone rod homeobox transcription factor) promoter, and human NRL promoter (neural retina leucine zipper transcription factor enhancer upstream of the human TK terminal promoter).

In some embodiments, a promoter of the present disclosure is suitable for use in AAV vectors. Sec, e.g., U.S. Patent Application Publication No. 2018/0155789, which is hereby incorporated by reference in its entirety for this purpose.

Non-limiting examples of constitutive promoters include CP1, CMV, EF1 alpha, SV40, PGK1, Ubc, human beta actin, beta tubulin, CAG, Ac5, Rosa26 promoter, COL1A1 promoter, polyhedrin, TEF1, GDS, CaM3 5S, Ubi, Hl, U6, red opsin promoter (red promoter), rhodopsin promoter (rho promoter), cone arrestin promoter (car promoter), rhodopsin kinase promoter (rk promoter). In some instances, the constitutive promoter is a Rosa26 promoter. In some instances, the constitutive promoter is a COL1A1 promoter. A tissue-specific promoter may be used to drive expression of an engineered nucleic acid, including e.g., a nucleic acid encoding a rtTA, (TA, OCT4, KLF4, SOX2, or any combination thereof. In some embodiments, a tissue-specific promoter is used to drive expression of a rtTA or a rTA. In some embodiments, a tissue-specific promoter is used to drive expression of OCT4, KLF4, and SOX2. In some embodiments, the SV40 promoter is used to drive expression of OCT4, KLF4, and SOX2.

“Tetracycline” refers to the tetracycline class of antibiotic compounds that includes, but is not limited to, tetracycline, chlortetracycline, oxytetracycline, demeclocyline, lymecycline, meclocycline, methacycline, minocycline, rolitetracycline, doxycycline, tigecycline, cravacycline, sarecycline, and omadacycline.

An “inducible promoter” is one that is characterized by initiating or enhancing transcriptional activity when in the presence of, influenced by, or contacted by an inducing agent. An inducing agent may be endogenous or a normally exogenous condition, compound, agent, or protein that contacts an engineered nucleic acid in such a way as to be active in inducing transcriptional activity from the inducible promoter. In certain embodiments, an inducing agent is a tetracycline-sensitive protein (e.g., tTA or rtTA, TetR family regulators).

Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (TetR, or TetRKRAB), a tetracycline operator sequence (tetO) and a tetracycline transactivator fusion protein (ITA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), pH-regulated promoters, and light-regulated promoters. A non-limiting example of an inducible system that uses a light-regulated promoter is provided in Wang et al, Nat. Methods. 2012 Feb. 12; 9(3):266-9.

In certain embodiments, an inducible promoter comprises a tetracycline (Tet)-responsive element. For example, an inducible promoter may be a TRE3G promoter (e.g., a TRE3G promoter that comprises a sequence that is at least 70% (e.g., at least 75%, 80%, 85%, 90%, 95%, 98%, 99%, or 100%) identical to SEQ ID NO: 7). As an example, a TRE (e.g., TRE2) promoter may comprise a nucleic acid sequence that is at least 70% (e.g., at least 75%, 80%, 85%, 90%, 95%, 98%, 99%, or 100%) identical to SEQ ID NO: 7.

Additional non-limiting examples of inducible promoters include mifepristone-responsive promoters (e.g., GAL4-Elb promoter) and coumermycin-responsive promoters. Sec, e.g., Zhao et al., Hum Gene Ther. 2003 Nov. 20; 14(17): 1619-29.

A “reverse tetracycline transactivator” (“rtTA”), as used herein, is an inducing agent that binds to a TRE promoter (e.g., a TRE3G, a TRE2 promoter, or a P tight promoter) in the presence of a tetracycline (e.g., doxycycline) and is capable of driving expression of a transgene that is operably linked to the TRE promoter. rtTAs generally comprise a mutant tetracycline repressor DNA binding protein (TetR) and a transactivation domain (see, e.g., Gossen et al, Science. 1995 Jun. 23; 268(5218): 1766-9 and any of the transactivation domains listed herein). The mutant TetR domain is capable of binding to a TRE promoter when bound to tetracycline. Sec, e.g., US Publ. Appl. No. 2021-0403923 A, and the International Publ. No. WO2020/069339, entitled MUTANT REVERSE TETRACYCLINE TRANSACTIVATORS FOR EXPRESSION OF GENES, each of which is herein incorporated by reference in its entirety.

“SRY-box 2” or “SOX2” is a member of the SRY-related HMG-box (SOX) family of transcription factors. SOX2 has been implicated in promoting embryonic development. Members of the SOX (SRY-related HMG-box) family of transcription factors are characterized by a high mobility group 5 (HMG)-box DNA sequence. This HMG box is a DNA binding domain that is highly conserved throughout eukaryotic species. A SOX2 transcription factor, homolog or variant thereof, as used herein, may be derived from any species, including humans. In certain embodiments, the nucleic acid molecule encoding SOX2 comprises a sequence that is at least 70% (e.g., at least 75%, 80%, 85%, 90%, 95%, 98%, 99%, or 100%) identical to a nucleic acid described in the NCBI RefSeq under accession number NM_01 1443.4. In certain embodiments, the nucleic acid molecule encoding a human SOX2 comprises a sequence that is at least 70% (e.g., at least 75%, 80%, 85%, 90%, 95%, 98%, 99%, or 100%) identical to a nucleic acid molecule described in the NCBI RefSeq under accession number NM_003106.4. SEQ ID NO: 3 is a non-limiting example of a nucleotide sequence encoding human SOX2. In certain embodiments, the nucleic acid molecule encoding human SOX2 comprises a sequence that is at least 70% (e.g., at least 75%, 80%, 85%, 90%, 95%, 98%, 99%, or 100%) identical to SEQ ID NO: 3. According to some embodiments, the nucleic acid molecule encodes a SOX2 comprising an amino acid sequence that is at least 70% (e.g., at least 75%, 80%, 85%, 90%, 95%, 98%, 99%, or 100%) identical to the amino acid sequence described in the NCBI RefSeq under accession number NP_003097.1. According to some embodiments, the nucleic acid molecule encodes a SOX2 comprising the amino acid sequence described in the NCBI RefSeq under accession number NP_003097.1. In some instances, the nucleic acid molecule encodes a SOX2 comprising an amino acid sequence that is at least 70% (e.g., at least 75%, 80%, 85%, 90%, 95%, 98%, 99%, or 100%) identical to SEQ ID NO: 4. SEQ ID NO: 4 is a non-limiting example of an amino acid sequence of human SOX2.

A “multicistronic vector” is a vector that encodes more than one amino acid sequence (e.g., a vector encoding OCT4 and KLF4, OCT4 and SOX2, KLF4 and SOX2, or OCT4, SOX2, and KL4 (OSK)). A multicistronic vector allows for expression of multiple amino acid sequences from a nucleic acid sequence. Nucleic acid sequences encoding each transcription factor (e.g., OCT4, KLF4, or SOX2) may be connected or separated such that they produce unconnected proteins. For example, internal ribosome entry sites (IRES) or polypeptide cleavage signals may be placed between nucleic acid sequences encoding each transcription factor in a vector. Exemplary polypeptide cleavage signals include 2A peptides (e.g., T2A, P2A, E2A, and F2A). A T2A peptide may comprise a sequence that is at least 70% (e.g., at least 75%, 80%, 85%, 90%, 95%, 98%, 99%, or 100%) identical to SEQ ID NO: 10. A P2A peptide may comprise a sequence that is at least 70% (e.g., at least 75%, 80%, 85%, 90%, 95%, 98%, 99%, or 100%) identical to SEQ ID NO: 8.

In some embodiments, an expression vector of the present disclosure is a multicistronic expression vector.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds, such as chickens, ducks, geese, and/or turkeys). In certain embodiments, the animal is a mammal. The animal may be a male or female and at any stage of development. A non-human animal may be a transgenic animal.

A “terminator” or “terminator sequence.” as used herein, is a nucleic acid (s, engineered nucleic acid) sequence that causes transcription to stop. A terminator may be unidirectional or bidirectional. It is comprised of a DNA sequence involved in specific termination of an RNA transcript by an RNA polymerase. A terminator sequence prevents transcriptional activation of downstream nucleic acid sequences by upstream promoters. Thus, in certain embodiments, a terminator that ends the production of an RNA transcript is contemplated.

The most commonly used type of terminator is a forward terminator. When placed downstream of a nucleic acid sequence that is usually transcribed, a forward transcriptional terminator will cause transcription to abort. In some embodiments, bidirectional transcriptional terminators may be used, which usually cause transcription to terminate on both the forward and reverse strand. In some embodiments, reverse transcriptional terminators may be used, which usually terminate transcription on the reverse strand only.

Non-limiting examples of mammalian terminator sequences include bovine growth hormone terminator, and viral termination sequences such as, for example, the SV40 terminator, spy, yejM, secG-leuU, thrLABC, rrnB Tl, hisLGDCBHAFI, metZWV, rmnC, xapR, aspA, and arcA terminator. In certain embodiments, the terminator sequence is SV40 and comprises a sequence that is at least 70% (e.g., at least 75%, 80%, 85%, 90%, 95%, 98%, 99%, or 100%) identical to SEQ ID NO: 12.

A “Tet-Off” system, as used herein, is a type of inducible system that is capable of repressing expression of a particular transgene in the presence of a tetracycline (e.g., doxycycline (DOX)). Conversely, a Tet-Off system is capable of inducing expression of a particular transgene in the absence of a tetracycline (e.g., doxycycline, DOX). In certain embodiments, a Tet-Off system comprises a tetracycline-responsive promoter operably linked to a transgene (e.g., encoding OCT4; KLF4; SOX2; or any combination thereof) and a tetracycline-controlled transactivator (ITA). The transgene with the tetracycline-responsive promoter (e.g., TRE3G, P tight, or TRE2) and the tetracycline-controlled transactivator may be encoded on the same vector or be encoded on separate vectors. Sec, e.g., US Publ. Appl. No. 2021-0403923 A, and the International Publ. No. WO2020/069339, entitled MUTANT REVERSE TETRACYCLINE TRANSACTIVATORS FOR EXPRESSION OF GENES, each of which is herein incorporated by reference in its entirety.

A “Tet-On” system, as used herein, is a type of inducible system that is capable of inducing expression of a particular transgene in the presence of a tetracycline (e.g., doxycycline (DOX)). In certain embodiments, a Tet-On system comprises a tetracycline-responsive promoter operably linked to a transgene (e.g., encoding OCT4; KLF4; SOX2; or any combination thereof) and a reverse tetracycline-controlled transactivator (rtTA). For example, the rtTA may be rtTA3, ItTA4, or variants thereof. In certain embodiments, a nucleic acid encoding rtTA3 comprises a sequence that is at least 70% (e.g., at least 75%, at least 80%, at least 85%, at least 90%, at least 95%, or at least 100%) identical to SEQ ID NO: 19. In certain embodiments, rtTA3 comprises an amino acid sequence that is at least 70% (e.g., at least 75%, at least 80%, at least 85%, at least 90%, at least 95%, at least 98%, at least 99%, or at least 100%) identical to SEQ ID NO: 20. In certain embodiments, a nucleic acid encoding rtTA4 comprises a sequence that is at least 70% (e.g., at least 75%, at least 80%, at least 85%, at least 90%, at least 95%, at least 98%, at least 99%, or at least 100%) identical to SEQ ID NO: 26. In certain embodiments, rtTA4 comprises an amino acid sequence that is at least 70% (e.g., at least 75%, at least 80%, at least 85%, at least 90%, at least 95%, at least 98%, at least 99%, or at least 100%) identical to SEQ ID NO: 27. The expression cassette encoding a tetracycline-responsive promoter (e.g., a promoter comprising a TRE, including TRE3G, P tight, and TRE2) and a reverse tetracycline-controlled transactivator may be encoded on the same vector or be encoded on separate vectors. Sec, e.g., US Publ. Appl. No. 2021-0403923 A, and the International Publ. No. WO2020/069339.

The term “tissue” refers to any biological tissue of a subject (including a group of cells, a body part, or an organ) or a part thereof, including blood and/or lymph vessels, which is the object to which a compound, particle, and/or composition as disclosed herein is delivered. A tissue may be an abnormal, damaged, or unhealthy tissue, which may need to be treated. A tissue may also be a normal or healthy tissue that is under a higher than normal risk of becoming abnormal or unhealthy, which may need to be prevented. In certain embodiments, the tissue is considered healthy but suboptimal for performance or survival in current or future conditions. In certain embodiments, the tissue is the central nervous system. In certain embodiments, the cell or tissue is from eye. In certain embodiments, the tissue is damaged (e.g., due to a congenital defect, an injury, an accident, or an iatrogenic injury), diseased, and/or aged. In certain embodiments, the tissue is a deep tissue that is reachable with a fiber optic probe.

As used herein, a “TRE promoter” is a promoter comprising a tetracycline-responsive element (TRE). As used herein, a TRE comprises at least one (e.g., at least 2, 3, 4, 5, 6, 7, 8, 9, 10, 11, 12, 13, 14, 15, 16, 17, 18, 19, or 20) Tet-O sequences. In some embodiments, a TRE promoter further comprises a minimal promoter located downstream of a tet-O sequence. A minimal promoter is a promoter that comprises the minimal elements of a promoter (e.g., TATA box and transcription initiation site), but is inactive in the absence of an upstream enhancer (e.g., sequences comprising Tet-O). As an example, a minimal promoter may be a minimal CMV promoter that comprises a sequence that is at least 70% (e.g., at least 75%, 80%, 85%, 90%, 95%, 98%, 99%, or 100%) identical to SEQ ID NO: 17 or 24. For example, a TRE promoter may be a TRE3G promoter (e.g., a TRE3G promoter that comprises a sequence that is at least 70% (e.g., at least 75%, 80%, 85%, 90%, 95%, 98%, 99%, or 100%) identical to SEQ ID NO: 7.

The term “tissue repair” in the context of damaged tissue refers to restoration of tissue architecture, function following tissue damage, or a combination thereof. Tissue repair includes tissue regeneration, cell growth, tissue replacement, and/or rewiring of existing tissue (reprogramming).

The term “tissue regeneration” refers to production of new tissue or cells within a tissue that are the same type as the tissue of interest (e.g., same type as the damaged tissue or cell). In some embodiments, the methods provided herein promote organ regeneration.

The term “tissue replacement” refers to production of a different type of tissue compared to the tissue of interest (e.g., connective tissue to replace damaged tissue).

As used herein, the terms “treatment,” “treat,” and “treating” refer to reversing, alleviating, delaying the onset of, or inhibiting the progress of a disease or disorder, or one or more symptoms thereof, as described herein. In certain embodiments, treatment may be administered after one or more symptoms have developed. In other embodiments, treatment may be administered in the absence of symptoms. In other embodiments, treatment may be administered to improve one or more of retinal perfusion, ocular pressure, thickness of retinal layers, survival of RGCs, retinal electrical response, macular nerve electrical response, optic nerve activity, retinal nerve light response, thickness of retinal layer (OCT), survival of retinal RGC cells, visual acuity, and the like. For example, treatment may be administered to a susceptible individual prior to the onset of symptoms. Treatment may also be continued after symptoms have resolved, for example, to prevent or delay their recurrence.

The term “variant” refers to a sequence that comprises a modification relative to a wild-type sequence. Non-limiting modifications in an amino acid sequence include insertions, deletions, and point mutations. Non-limiting modifications to nucleic acid sequences include frameshift mutations, nucleotide insertions, and nucleotide deletions.

The term “WPRE” refers to a Woodchuck Hepatitis Virus (WHP) Posttranscriptional Regulatory Element (WPRE). WPREs create tertiary structures in nucleic acids (e.g., expression vectors) and are capable of enhancing transgene expression (e.g., from a viral vector). In certain embodiments, a WPRE sequence is at least 70% (e.g., at least 75%, at least 80%, at least 85%, at least 90%, at least 95%, at least 98%, at least 99%, or at least 100%) identical to SEQ ID NO: 23 or 31.

### Nucleic Acid Molecules

The present disclosure provides nucleic acid molecules that include a nucleic acid sequence encoding OCT4, a nucleic acid sequence encoding SOX2, a nucleic acid sequence encoding KLF4, or any combination thereof, and in the absence of an exogenous nucleic acid sequence encoding c-Myc. The nucleic acid molecule may be a vector, including for example an expression vector. In certain embodiments, the nucleic acid molecule includes a nucleic acid sequence encoding OCT4. In certain embodiments, the nucleic acid molecule includes a nucleic acid sequence encoding SOX2. In certain embodiments, the nucleic acid molecule includes a nucleic acid sequence encoding KLF4. In certain embodiments, the nucleic acid molecule includes any two of a nucleic acid sequence encoding OCT4, a nucleic acid sequence encoding SOX2, and a nucleic acid sequence encoding KLF4. In certain embodiments, the nucleic acid molecule includes a first nucleic acid sequence encoding OCT4, a second nucleic acid sequence encoding SOX2, and a third nucleic acid sequence encoding KLF4. In certain embodiments, OCT4 comprises an amino acid sequence at least 70% (e.g., at least 75%, 80%, 85%, 90%, 95%, 98%, 99%, or 100%) identical to SEQ ID NO: 2. In certain embodiments, the nucleic acid sequence encoding OCT4 is at least 70% (e.g., at least 75%, 80%, 85%, 90%, 95%, 98%, 99%, or 100%) identical to SEQ ID NO: 1. In certain embodiments, SOX2 comprises an amino acid sequence at least 70% (e.g., at least 75%, 80%, 85%, 90%, 95%, 98%, 99%, or 100%) identical to SEQ ID NO: 4. In certain embodiments, the nucleic acid sequence encoding SOX2 is at least 70% (e.g., at least 75%, 80%, 85%, 90%, 95%, 98%, 99%, or 100%) identical to SEQ ID NO: 3. In certain embodiments, KLF4 comprises an amino acid sequence at least 70% (e.g., at least 75%, 80%, 85%, 90%, 95%, 98%, 99%, or 100%) identical to SEQ ID NO: 6. In certain embodiments, the nucleic acid sequence encoding KLF4 is at least 70% identical (e.g., at least 75%, 80%, 85%, 90%, 95%, 98%, 99%, or 100%) to SEQ ID NO: 5. In certain embodiments, OCT4, SOX2, KLF4, or any combination thereof is a human protein. In certain embodiments, OCT4, SOX2, KLF4, or any combination thereof is a non-human protein, for example, a protein from one or more mammals including from one or more primates (e.g., cynomolgus monkeys, rhesus monkeys). If two or more of OCT4, SOX2, and KLF4 are on one nucleic acid molecule, they may be in any order. The words “first,” “second,” and “third” do not necessarily imply an order of the genes on the nucleic acid molecule.

In some embodiments, the nucleic acid molecule of the present disclosure includes an inducible promoter. In some embodiments, the nucleic acid molecule has one inducible promoter. In such instances, the expression of OCT4, SOX2, and KLF4 are under the control of the same inducible promoter. In some embodiments, the nucleic acid molecule has more than one inducible promoter. The inducible promoter may include a tetracycline-responsive element (TRE) (e.g., a TRE3G promoter, a TRE2 promoter, or a P tight promoter), mifepristone-responsive promoters (e.g., GAL4-Elb promoter), or a coumermycin-responsive). As an example, a TRE (e.g., TRE3G) promoter may comprise a nucleic acid sequence that is at least 70% (e.g., at least 75%, 80%, 85%, 90%, 95%, 98%, 99%, or 100%) identical to SEQ ID NO: 7. Sec, e.g., US Publ. Appl. No. 2021-0403923 A. and the International Publ. No. WO2020/069339.

In certain embodiments, the inducing agent is capable of inducing expression of the first (e.g., OCT4), second (e.g., SOX2), third (e.g., KLF4) nucleic acids, or any combination thereof from the inducible promoter in the presence of a tetracycline (e.g., doxycycline). In certain embodiments, the inducing agent is reverse tetracycline-controlled transactivator (rtTA) (e.g., M2-rtTA, rtTA3 or rtTA4). In certain embodiments, the rtTA is rtTA3 comprising an amino acid sequence that is at least 70% (e.g., at least 75%, 80%, 85%, 90%, 95%, 98%, 99%, or 100%) identical to SEQ ID NO: 20. In certain embodiments, rtTA3 is encoded by a nucleic acid sequence that is at least 70% (e.g., at least 75%, 80%, 85%, 90%, 95%, 98%, 99%, or 100%) identical to SEQ ID NO: 19. In certain embodiments, the rtTA is rtTA4 and comprises an amino acid sequence that is at least 70% (e.g., at least 75%, 80%, 85%, 90%, 95%, 98%, 99%, or 100%) identical to SEQ ID NO: 27. In certain embodiments, rtTA4 is encoded by a nucleic acid sequence that is at least 70% (e.g., at least 75%, 80%, 85%, 90%, 95%, 98%, 99%, or 100%) identical to SEQ ID NO: 26.

In certain embodiments, the inducing agent is capable of inducing expression of expression of the first nucleic acid (e.g., OCT4), second nucleic acid (e.g., SOX2), third nucleic (e.g., KLF4), or any combination thereof from the inducible promoter in the absence of a tetracycline (e.g., doxycycline).

In certain embodiments, the inducing agent is tetracycline-controlled transactivator ((TA).

In certain embodiments, the nucleic acid molecule of the present disclosure comprises a constitutive promoter, for example, one or more of CP1, CMV, EF1 alpha, SV40, PGK1, Ubc, human beta actin, CAG, Ac5, polyhedrin, TEF1, GDS, CaM3 5S, Ubi, Hl, and/or U6 promoter. The constitutive promoter may be operably linked to nucleic acid sequences encoding OCT4, KLF4, SOX2, an inducing agent, or a combination thereof. In some embodiments, the nucleic acid molecule comprises one constitutive promoter. In some embodiments, the nucleic acid molecule comprises more than one constitutive promoter.

In certain embodiments, the nucleic acid molecule of the present disclosure comprises an SV40-derived terminator sequence. In certain embodiments, the SV40-derived sequence is at least 70% (e.g., at least 75%, 80%, 85%, 90%, 95%, 98%, 99%, or 100%) identical to SEQ ID NO: 12 or 30.

In certain embodiments, the nucleic acid molecule of the present disclosure comprises a separator sequence, which may be useful in producing two separate amino acid sequences from one transcript. The separator sequence may encode a self-cleaving peptide (e.g., 2A peptide, including a 2A peptide sequence that is at least 70% (e.g., at least 75%, 80%, 85%, 90%, 95%, 98%, 99%, or 100%) identical to SEQ ID NO: 8 or 10). In certain embodiments, the separator sequence is an Internal Ribosome Entry Site (IRES).

In certain embodiments, the nucleic acid molecule is a viral vector (e.g., a lentiviral, a retroviral, or an adeno-associated virus (AAV) vector). An AAV vector of the present disclosure generally comprises inverted terminal repeats (ITRs) flanking a transgene of interest (e.g., a nucleic acid sequence encoding OCT4, SOX2, KLF4, an inducing agent, or a combination thereof). In some embodiments, the distance between two inverted terminal repeats is less than 5.0 kilobases(kb) (e.g., less than 4.9 kb, less than 4.8 kb, less than 4.7 kb, less than 4.6 kb, less than 4.5 kb, less than 4.4 kb, less than 4.3 kb, less than 4.2 kb, less than 4.1 kb, less than 4 kb, less than 3.5 kb, less than 3 kb, less than 2.5 kb, less than 2 kb, less than 1.5 kb, less than 1 kb, or less than 0.5 kb).

In certain embodiments, the nucleic acid molecule (e.g., an expression vector encoding OCT4, KLF4, SOX2, an inducing agent, or a combination thereof) of the present disclosure may further comprise a nucleic acid sequence encoding a selection agent (e.g., an antibiotic, including blasticidin, geneticin, hygromycin B, mycophenolic acid, puromycin, zeocin, actinomycin D, ampicillin, carbenicillin, kanamycin, and neomycin) and/or detectable marker (e.g., GFP, RFP, luciferase, CFP, mCherry, DsRed2FP, mKate, biotin, FLAG-tag, HA-tag, His-tag, Myc-tag, V5-tag, etc.).

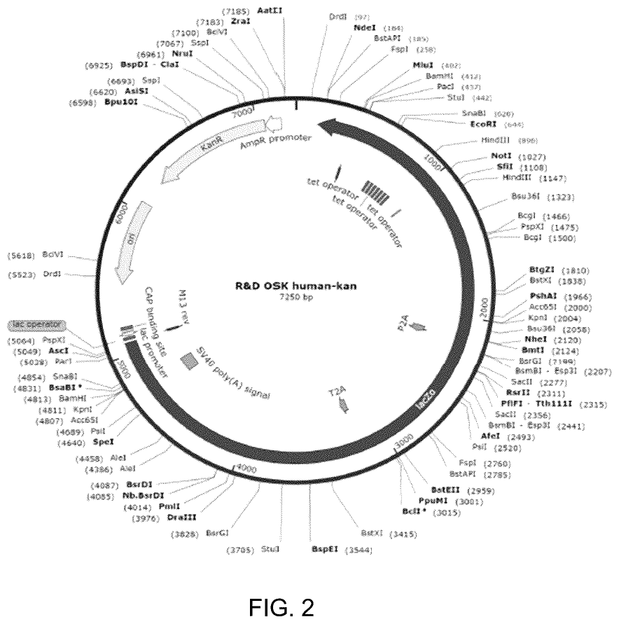

In some embodiments, the expression vector encoding OCT4, KLF4, and SOX2 comprises the sequence provided in SEQ ID NO: 13, SEQ ID NO: 14, or SEQ ID NO: 15. In some embodiments, the expression vector encoding OCT4, KLF4, and SOX2 comprises the elements depicted in FIG. 2, or a combination thereof. The expression vector may be a viral vector. The viral vector may be an adeno-associated virus (AAV) vector, retroviral vector, lentiviral vector, herpes viral vector, and the like.

In another aspect, the present disclosure provides recombinant viruses. The recombinant viruses can include one or more lentivirus, adenovirus, retrovirus, herpes virus, alphavirus, vaccinia virus or adeno-associated virus (AAV) comprising any of the expression vectors described herein. In certain embodiments, the recombinant virus encodes a transcription factor selected from OCT4; KLF4; SOX2; and any combination thereof. In certain embodiments, the recombinant virus encodes two or more transcription factors selected from the group consisting of OCT4, KLF4, and SOX2. In certain embodiments, the recombinant virus encodes OCT4 and SOX2, OCT4 and KLF4, or SOX2 and KLF4. In certain embodiments, the recombinant virus encodes OCT4, KLF4, and SOX2. In certain embodiments, the recombinant virus encodes four or more transcription factors, for example OCT4, SOX2, KLF4, and another transcription factor.

### Methods of Treatment of NAION

Further provided herein are methods for treating non-arteritic anterior ischemic optic neuropathy (NAION) in a subject in need thereof wherein the method includes administering to the subject one or more agents for upregulating OCT4, SOX2, KLF4, and/or one or more combinations thereof to the subject. The one or more agents do not upregulate c-Myc. The one or more agents for upregulating OCT4, SOX2, KLF4 (OSK) may include one or more means for inducing expression of OSK, including DNA, RNA, small molecules, and the like. In some embodiments, the methods include administering to the subject one or more nucleic acid molecules as contemplated herein. In some embodiments the one or more nucleic acid molecules include a nucleic acid molecule system having at least two nucleic acid molecules.

In some embodiments of the methods for treating NAION in a subject, the agent for upregulating OSK expression includes at least one nucleic acid molecule encoding OSK as described herein above. The nucleic acid molecule encoding OSK does not comprise a nucleic acid sequence encoding c-myc. The nucleic acid molecule encoding OSK may be an adeno-associated viral (AAV) vector. According to some embodiments, the methods further comprise administering to the subject a nucleic acid molecule encoding a reverse tetracycline-controlled transactivator (rtTA). The nucleic acid molecule encoding a reverse tetracycline-controlled transactivator (rtTA) may be an AAV vector. In some embodiments, the AAV vector comprising a nucleic acid molecule encoding reverse tetracycline-controlled transactivator (rtTA) is not the same AAV vector as the AAV vector comprising a nucleic acid molecule encoding OCT4, SOX2, and KLF4.

The nucleic acid molecule encoding OCT4, SOX2, and KLF4 is operably linked to an inducible promoter. In some embodiments, the inducible promoter is induced by a tetracycline class antibiotic. Tetracycline class antibiotics are known in the art and include, for example, tetracycline, chlortetracycline, oxytetracycline, demeclocyline, lymecycline, meclocycline, methacycline, minocycline, rolitetracycline, doxycycline, tigecycline, cravacycline, sarecycline, and omadacycline. Doxycycline is an exemplary tetracycline class antibiotic. In some embodiments, the inducible promoter is a tetracycline class antibiotic response element (TRE) including for example a TRE2 promoter.

The reverse tetracycline-controlled transactivator (rtTA) may be rtTA3, rtTA4, or combinations thereof.

In some embodiments, the nucleic acid molecule encoding rtTA is operably linked to a constitutive promotor including one or more of CP1, CMV, EF1 alpha, SV40, PGK1, Ubc, human beta actin, CAG, Ac5, polyhedrin, TEF1, GDS, CaM3 5S, Ubi, Hl, and/or U6 promoter. In some embodiments, the nucleic acid molecule encoding rtTA is operably linked a CMV promoter.

In some embodiments, the AAV vector is serotype-2 (AAV2). In some embodiments, the AAV vector is a hybrid vector comprising capsid proteins from one or more serotypes including AAV1, AAV2, AAV5, AAV6, AAV7, AAV8 and AAV9 (e.g., AAV2/2, AAV2/6. AAV2/1, AAV2/5, AAV2/7, AAV2/8 and AAV2/9).

In some embodiments, the AAV vector comprising a nucleic acid molecule encoding OCT4, SOX2, and KLF4 comprises a self-cleaving peptide, for example a 2A peptide.

In some embodiments, the AAV vector comprising a nucleic acid molecule encoding OCT4, SOX2, and KLF4 comprises inverted terminal repeats (ITRs) flanking the first nucleic acid. In some embodiments, the AAV vector comprising a nucleic acid molecule encoding OCT4, SOX2, and KLF4 comprises inverted terminal repeats (ITRs) flanking the second nucleic acid. In some embodiments, the AAV vector comprising a nucleic acid molecule encoding OCT4, SOX2, and KLF4 comprises inverted terminal repeats (ITRs) flanking the third nucleic acid. In some embodiments, the AAV vector comprising a nucleic acid molecule encoding OCT4, SOX2, and KLF4 comprises inverted terminal repeats (ITRs) flanking one or more combinations of the first nucleic acid, the second nucleic acid, and/or the third nucleic acid. In some embodiments, the distance between two inverted terminal repeats (ITRs) is less than 5.0 kilobases(kb) (e.g., less than 4.9 kb, less than 4.8 kb, less than 4.7 kb, less than 4.6 kb, less than 4.5 kb, less than 4.4 kb, less than 4.3 kb, less than 4.2 kb, less than 4.1 kb, less than 4 kb, less than 3.5 kb, less than 3 kb, less than 2.5 kb, less than 2 kb, less than 1.5 kb, less than 1 kb, or less than 0.5 kb). In some embodiments, the distance between two ITRs is 4.7 kb or less.

The method can further include administering an inducing agent to the subject. The inducing agent can include for example a tetracycline-controlled transactivator ((TA). In certain aspects, the inducing agent is capable of inducing expression of expression of the first nucleic acid (e.g., OCT4), the second nucleic acid (e.g., SOX2), the third nucleic (e.g., KLF4), or any combination thereof from the inducible promoter in the absence of a tetracycline (e.g., doxycycline).

In some embodiments, the AAV-OSK vector comprising a nucleic acid molecule encoding OCT4, SOX2, and KLF4 comprises nucleic acid elements in a specific order. For example, the AAV vector comprising a nucleic acid molecule encoding OCT4, SOX2, and KLF4 can include elements in the following order: a) a first inverted terminal repeat sequence (ITR) sequence; b) a TRE2 promoter sequence; c) an OCT4 sequence; d) a P2A cleavage sequence; c) a SOX2 sequence; f) a T2A cleavage sequence; g) a KLF4 sequence; h) an SV-40-derived terminator sequence; and i) a second inverted terminal repeat (ITR) sequence, as described, for example in U.S. patent application Ser. No. 17/280,384, published as Intl. Publ. No. WO2020/069373 titled CELLULAR REPROGRAMMING TO REVERSE AGING AND PROMOTE ORGAN AND TISSUE REGENERATION, incorporated by reference herein in its entirety.

In certain embodiments, the encoded OCT4 comprises an amino acid sequence at least 70% (e.g., at least 75%, 80%, 85%, 90%, 95%, 98%, 99%, or 100%) identical to SEQ ID NO: 2. In certain embodiments, the nucleic acid sequence encoding OCT4 is at least 70% (e.g., at least 75%, 80%, 85%, 90%, 95%, 98%, 99%, or 100%) identical to SEQ ID NO: 1. In certain embodiments, the encoded SOX2 comprises an amino acid sequence at least 70% (e.g., at least 75%, 80%, 85%, 90%, 95%, 98%, 99%, or 100%) identical to SEQ ID NO: 4. In certain embodiments, the nucleic acid sequence encoding SOX2 is at least 70% (e.g., at least 75%, 80%, 85%, 90%, 95%, 98%, 99%, or 100%) identical to SEQ ID NO: 3. In certain embodiments, the encoded KLF4 comprises an amino acid sequence at least 70% (e.g., at least 75%, 80%, 85%, 90%, 95%, 98%, 99%, or 100%) identical to SEQ ID NO: 6. In certain embodiments, the nucleic acid sequence encoding KLF4 is at least 70% identical (e.g., at least 75%, 80%, 85%, 90%, 95%, 98%, 99%, or 100%) to SEQ ID NO: 5. In some embodiments, the nucleic acid sequence encoding OCT4 is SEQ ID NO: 1, the nucleic acid sequence encoding SOX2 is SEQ ID NO: 3, and the nucleic acid sequence encoding KLF4 is SEQ ID NO: 5.

In some embodiments, the P2A sequence encodes for a polypeptide with the sequence ATNFSLLKQAGDVEENPGP (SEQ ID NO: 9). In some embodiments, the P2A sequence is

(SEQ ID NO: 8)
GCCACGAACTTCTCTCTGTTAAAGCAAGCAGGAGATGTTGAAGAAAACCC
CGGGCCT.

In some embodiments, the T2A sequence encodes a polypeptide of SEQ ID NO: 11. In some embodiments, the T2A sequence is

(SEQ ID NO: 10)
GAGGGCAGGGGAAGTCTTCTAACATGCGGGGACGTGGAGGAAAATCCCGG
CCCA.

In some embodiments, the TRE2 promoter sequence is SEQ ID NO: 7. In some embodiments, the TRE2 promoter sequence comprises at least one minimal CMV promoter sequence. In some embodiments, the at least one minimal SV40 promoter sequence is SEQ ID NO: 12.

In some embodiments, the SV-40-derived terminator sequence is SEQ ID NO: 12.

In some embodiments, the ITR sequence is SEQ ID NO: 16.

In some embodiments, the nucleic acid molecule encoding OCT4, SOX2, and KLF4 comprises SEQ ID NO: 13 or 14. In some embodiments, the nucleic acid molecule encoding encoding OCT4, SOX2, and KLF4 comprises SEQ ID NO: 15.

The AAV vector comprising a nucleic acid molecule encoding OCT4, SOX2, and KLF4 and the AAV vector comprising a nucleic acid molecule encoding rtTA are administered sequentially or simultaneously.

The nucleic acid molecules disclosed herein can be administered to a subject by any appropriate route including, without limitation, intravenous, intraperitoneal, subcutaneous, intramuscular, intranasal, topical, or intradermal routes. In certain embodiments, the composition is formulated for administration via intravenous injection or subcutaneous injection. In some embodiments, the dual vector system including an AAV vector comprising a nucleic acid molecule encoding OCT4, SOX2, and KLF4 and an AAV vector comprising a nucleic acid molecule encoding rtTA is administered intravitreally.

In some embodiments, the nucleic acid molecule encoding OCT4, SOX2, and KLF4 and the nucleic acid molecule encoding rtTA (e.g., rtTA3, rtTA4, etc.) are administered at a ratio (nucleic acid molecule:nucleic acid molecule) of about 1:20, 1:19, 1:18, 1:17, 1:16, 1:15, 1:14, 1:13, 1:12, 1:11, 1:10; 1:9; 1:8; 1:7; 1:6; 1:5; 1:4; 1:3; 1:2; 1:1; 1:0.5; 2:1; 3:1; 4:1; 5:1; 6:1, 7:1; 8:1; 9:1; 10:1, 11:1, 12:1, 13:1, 14:1, 15:1, 16:1, 17:1, 18:1, 19:1, or 20:1. In some embodiments, the nucleic acid molecule encoding OCT4, SOX2, and KLF4 and the nucleic acid molecule encoding rtTA (e.g., rtTA3, rtTA4, etc.) are administered at a ratio of about 1:2; 1:1.9; 1:1.8; 1:1.7; 1:1.6; 1:1.5; 1:1.4; 1:1.3; 1:1.2; 1:1.1; 1:1; 1:0.9; 1:0.8; 1:0.7; 1:0.6; 1:0.5; 1:0.4; 1:0.3; 1:0.2; 1:0.1; 0.1:1; 0.2:1; 0.3:1; 0.4:1; 0.5:1, 0.6:1; 0.7:1; 0.8:1; 0.9:1; 1:1; 1.1:1; 1.2:1; 1.3:1; 1.4:1; 1.5:1; 1.6:1; 1.7:1; 1.8:1; 1.9:1 or 2:1. In some embodiments, the nucleic acid molecule encoding OCT4, SOX2, and KLF4 and the nucleic acid molecule encoding rtTA are administered at an about 1:1 ratio.

In some embodiments, the AAV vector comprising a nucleic acid molecule encoding OCT4, SOX2, and KLF4 and the AAV vector comprising a nucleic acid molecule encoding rtTA (e.g., rtTA3, rtTA4, etc.) are administered at a ratio (vector genome:vector genome (vg:vg)) of about 1:20, 1:19, 1:18, 1:17, 1:16, 1:15, 1:14, 1:13, 1:12, 1:11, 1:10; 1:9; 1:8; 1:7; 1:6; 1:5; 1:4; 1:3; 1:2; 1:1; 1:0.5; 2:1; 3:1; 4:1; 5:1; 6:1, 7:1; 8:1; 9:1; 10:1, 11:1, 12:1, 13:1, 14:1, 15:1, 16:1, 17:1, 18:1, 19:1, or 20:1. In some embodiments, the AAV vector comprising a nucleic acid molecule encoding OCT4, SOX2, and KLF4 and the AAV vector comprising a nucleic acid molecule encoding rtTA are administered at an about 1:1 (vg:vg) ratio. In some embodiments, the AAV vector comprising a nucleic acid molecule encoding OCT4, SOX2, and KLF4 and the AAV vector comprising a nucleic acid molecule encoding rtTA (e.g., rtTA3, rtTA4, etc.) are administered at a ratio (vg/vg) of about 1:10; 1:9; 1:8; 1:7; 1:6; 1:5; 1:4; 1:3; 1:2; 1:1; 1:0.5; 2:1; 3:1; 4:1; 5:1; 6:1, 7:1; 8:1; 9:1; or 10:1. In some embodiments, the AAV vector comprising a nucleic acid molecule encoding OCT4, SOX2, and KLF4 and the AAV vector comprising a nucleic acid molecule encoding rtTA (e.g., rtTA3, rtTA4, etc.) are administered at a ratio (vg/vg) of about 1:2; 1:1.9; 1:1.8; 1:1.7; 1:1.6; 1:1.5; 1:1.4; 1:1.3; 1:1.2; 1:1.1; 1:1; 1:0.9; 1:0.8; 1:0.7; 1:0.6; 1:0.5; 1:0.4; 1:0.3; 1:0.2; 1:0.1; 0.1:1; 0.2:1; 0.3:1; 0.4:1; 0.5:1, 0.6:1; 0.7:1; 0.8:1; 0.9:1; 1:1; 1.1:1; 1.2:1; 1.3:1; 1.4:1; 1.5:1; 1.6:1; 1.7:1; 1.8:1; 1.9:1 or 2:1. In some embodiments, the AAV vector comprising a nucleic acid molecule encoding OCT4, SOX2, and KLF4 and the AAV vector comprising a nucleic acid molecule encoding rtTA are administered at an about 1:1 (vg/vg) ratio. According to some embodiments of the disclosed methods, the AAV vector comprising a nucleic acid molecule encoding OCT4, SOX2, and KLF4 comprises SEQ ID NO: 13, 14, or 35. According to some embodiments, the AAV vector comprising a nucleic acid molecule encoding rtTA comprises SEQ ID NO: 19, 26, 36, or 37. According to some embodiments of the disclosed methods, the AAV vector comprising a nucleic acid molecule encoding OCT4, SOX2, and KLF4 comprises SEQ ID NO: 13, 14, or 35 and the AAV vector comprising a nucleic acid molecule encoding rtTA comprises SEQ ID NO: 19, 26, 36, or 37. According to some embodiments of the disclosed methods, the AAV vector comprising a nucleic acid molecule encoding OCT4, SOX2, and KLF4 comprises SEQ ID NO: 35 and the AAV vector comprising a nucleic acid molecule encoding rtTA comprises SEQ ID NO: 36 or 37.

In some embodiments, the AAV vector comprising a nucleic acid molecule encoding OCT4, SOX2, and KLF4 is an AAV2-TRE-OSK vector and the AAV vector comprising a nucleic acid molecule encoding rtTA is an AAV2-CMV-rtTA3. The AAV composition may include an AAV2-TRE-OSK vector and an AAV2-CMV-rtTA3 vector. The methods may include the AAV2-TRE-OSK vector and the AAV2-CMV-rtTA3 vector in the same or in separate compositions.

In some embodiments, the AAV vector comprising a nucleic acid molecule encoding OCT4, SOX2, and KLF4 is an AAV2-TRE-OSK vector and the AAV vector comprising a nucleic acid molecule encoding rtTA is an AAV2-CMV-rtTA4. The AAV composition may include an AAV2-TRE-OSK vector and an AAV2-CMV-rtTA4 vector. The methods may include the AAV2-TRE-OSK vector and the AAV2-CMV-rtTA4 vector in the same composition or in separate compositions.

The concentration of the AAV2-TRE-OSK vector to be administered in accordance with the disclosed methods includes an amount in the range of from about 1× 10¹⁰ vg/mL to about 2×10¹³ vg/mL, for example, 1×10¹² vg/mL to about 2×10¹² vg/mL. For example, the effective amount of the AAV2-TRE-OSK vector can include from about 1.0×10¹² vg/mL to about 1.1×10¹² vg/mL, from about 1.1×10¹² vg/mL to about 1.2×10¹² vg/mL, from about 1.2×10¹² vg/mL to about 1.3×10¹² vg/mL, from about 1.3×10¹² vg/mL to about 1.4×10¹² vg/mL, from about 1.4×10¹² vg/mL to about 1.5×10¹² vg/mL, from about 1.5×10¹² vg/mL to about 1.6×10¹² vg/mL, from about 1.6×10¹² vg/mL to about 1.7×10¹² vg/mL, from about 1.7×10¹² vg/mL to about 1.8×10¹² vg/mL, from about 1.8×10¹² vg/mL to about 1.9×10¹² vg/mL, from about 1.9×10¹² vg/mL to about 2.0×10¹² vg/mL and any and all increments therebetween.

The concentration of the AAV2-CMV-rtTA3 vector administered in accordance with the disclosed methods includes an amount in the range of from about 1×10¹⁰ vg/mL to about 2×10¹³ vg/mL, for example, about 1×10¹³ vg/mL to about 2×10¹³ vg/mL. For example, the effective amount of the AAV2-CMV-rtTA3 vector can include from about 1.0×10¹³ vg/mL to about 1.1×10¹³ vg/mL, from about 1.1×10¹³ vg/mL to about 1.2×10¹³ vg/mL, from about 1.2×10¹³ vg/mL to about 1.3×10¹³ vg/mL, from about 1.3×10¹³ vg/mL to about 1.4×10¹³ vg/mL, from about 1.4×10¹³ vg/mL to about 1.5×10¹³ vg/mL, from about 1.5×10¹³ vg/mL to about 1.6×10¹³ vg/mL, from about 1.6×10¹³ vg/mL to about 1.7×10¹³ vg/mL, from about 1.7×10¹³ vg/mL to about 1.8×10¹³ vg/mL, from about 1.8×10¹³ vg/mL to about 1.9×10¹³ vg/mL, from about 1.9×10¹³ vg/mL to about 2.0×10¹³ vg/mL and any and all increments therebetween.

The concentration of the AAV2-CMV-rtTA4 vector administered in accordance with the disclosed methods includes an amount in the range of from about 1×10¹⁰ vg/mL to about 2×10¹³ vg/mL, for example, about 1×10¹³ vg/mL to about 2×10¹³ vg/mL. For example, the effective amount of the AAV2-CMV-rtTA4 vector can include from about 1.0×10¹³ vg/mL to about 1.1×10¹³ vg/mL, from about 1.1×10¹³ vg/mL to about 1.2×10¹³ vg/mL, from about 1.2×10¹³ vg/mL to about 1.3×10¹³ vg/mL, from about 1.3×10¹³ vg/mL to about 1.4×10¹³ vg/mL, from about 1.4×10¹³ vg/mL to about 1.5×10¹³ vg/mL, from about 1.5×10¹³ vg/mL to about 1.6×10¹³ vg/mL, from about 1.6×10¹³ vg/mL to about 1.7×10¹³ vg/mL, from about 1.7×10¹³ vg/mL to about 1.8×10¹³ vg/mL, from about 1.8×10¹³ vg/mL to about 1.9×10¹³ vg/mL, from about 1.9×10¹³ vg/mL to about 2.0×10¹³ vg/mL and any and all increments therebetween.

According to some embodiments, the effective dose of the AAV2-TRE-OSK vector administered in accordance with the disclosed methods includes the amount of vector administered per eye. The effective dose can include an amount of vector in the range of from about 1×10⁹ vg/eye to about 1×10¹⁴ vg/eye. In some embodiments, the effective dose of the AAV2-TRE-OSK vector can include an amount of vector in the range of from about 1×10¹¹ vg/eye to about 10×10¹¹ vg/eye. For example, the dose of the AAV2-TRE-OSK vector can include from about 1×10¹¹ vg/eye to about 2×10¹¹ vg/eye, from about 2×10¹¹ vg/eye to about 3×10¹¹ vg/eye, from about 3×10¹¹ vg/eye to about 4×10¹¹ vg/eye, from about 4×10¹¹ vg/eye to about 5×10¹¹ vg/eye, from about 5×10¹¹ vg/eye to about 6×10¹¹ vg/eye, from about 6×10¹¹ vg/eye to about 7×10¹¹ vg/eye, from about 7×10¹¹ vg/eye to about 8×10¹¹ vg/eye, from about 8×10¹¹ vg/eye to about 9×10¹¹ vg/eye, from about 9×10¹¹ vg/eye to about 10×10¹¹ vg/eye, including any and all increments therebetween. In some embodiments, the effective dose of the AAV2-TRE-OSK vector is about 3.06×10¹¹vg/eye.

The effective dose of the AAV2-CMV-rtTA3 vector administered in accordance with the disclosed methods includes the amount of vector administered per eye. The effective dose the AAV2-CMV-rtTA3 vector can include an amount of vector in the range of from about 1×10⁹ vg/eye to about 1×10¹⁴ vg/eye. In some embodiments, the effective dose the AAV2-CMV-rtTA3 vector can include an amount of vector in the range of from about 1×10¹¹ vg/eye to about 10×10¹¹ vg/eye. For example, the dose the AAV2-CMV-rtTA3 vector can include from about 1×10¹¹ vg/eye to about 2×10¹¹ vg/eye, from about 2×10¹¹ vg/eye to about 3×10¹¹ vg/eye, from about 3×10¹¹ vg/eye to about 4×10¹¹ vg/eye, from about 4×10¹¹ vg/eye to about 5×10¹¹ vg/eye, from about 5×10¹¹ vg/eye to about 6×10¹¹ vg/eye, from about 6×10¹¹ vg/eye to about 7×10¹¹ vg/eye, from about 7×10¹¹ vg/eye to about 8×10¹¹ vg/eye, from about 8×10¹¹ vg/eye to about 9×10¹¹ vg/eye, from about 9×10¹¹ vg/eye to about 10×10¹¹ vg/eye, including any and all increments therebetween. In some embodiments, the effective dose of the AAV2-CMV-rtTA3 vector is about 2.66×10¹¹ vg/eye.

The effective dose of the AAV2-CMV-rtTA4 vector administered in accordance with the disclosed methods includes the amount of vector injected per eye. The effective dose can include an amount of vector in the range of from about 1×10⁹ vg/eye to about 1×10¹⁴ vg/eye. In some embodiments, the effective dose the AAV2-CMV-rtTA4 vector can include an amount of vector in the range of from about 1×10¹¹ vg/eye to about 10×10¹¹ vg/eye. For example, the dose the AAV2-CMV-rtTA4 vector can include from about 1×10¹¹ vg/eye to about 2×10¹¹ vg/eye, from about 2×10¹¹ vg/eye to about 3×10¹¹ vg/eye, from about 3×10¹¹ vg/eye to about 4×10¹¹ vg/eye, from about 4×10¹¹ vg/eye to about 5×10¹¹ vg/eye, from about 5×10¹¹ vg/eye to about 6×10¹¹ vg/eye, from about 6×10¹¹ vg/eye to about 7×10¹¹ vg/eye, from about 7×10¹¹ vg/eye to about 8×10¹¹ vg/eye, from about 8×10¹¹ vg/eye to about 9×10¹¹ vg/eye, from about 9×10¹¹ vg/eye to about 10×10¹¹ vg/eye, including any and all increments therebetween. In some embodiments, the effective dose of the AAV2-CMV-rtTA4 vector is about 2.66×10¹¹ vg/eye.

Embodiments of the methods include administering the AAV composition comprising an AAV vector including a nucleic acid molecule encoding OCT4, SOX2, and KLF4 and an AAV vector including a nucleic acid molecule encoding rtTA to the subject by one or more suitable routes including oculus sinister (OS) injection, oculus dexter (OD) injection, or oculus uterque (OU) injection. Administration may include one or more injections, for example, administration may include one injection comprising the AAV composition comprising an AAV vector including a nucleic acid molecule encoding OCT4, SOX2, and KLF4 and the AAV vector including a nucleic acid molecule encoding rtTA. In other aspects, the administration may include two (or more) injections, for example, one injection comprising the AAV composition comprising an AAV vector including a nucleic acid molecule encoding OCT4, SOX2, and KLF4 and one injection comprising the AAV vector including a nucleic acid molecule encoding rtTA. The injection comprising the AAV vector including a nucleic acid molecule encoding OCT4, SOX2, and KLF4 may be administered and the injection comprising the AAV vector including a nucleic acid molecule encoding rtTA may be administered simultaneously or sequentially. For example, the injection comprising the AAV composition comprising an AAV vector including a nucleic acid molecule encoding OCT4, SOX2, and KLF4 may be administered before, at the same time as, or after the injection comprising the AAV vector including a nucleic acid molecule encoding rtTA.

Embodiments of the methods further include administering to the subject an effective amount of an antibiotic. In some embodiments, the antibiotic includes tetracycline or doxycycline. In some embodiments, the antibiotic is administered at least one day prior to administering the AAV vector comprising a nucleic acid molecule encoding OCT4, SOX2, and KLF4 and the AAV vector comprising a nucleic acid molecule encoding rtTA.

The antibiotic may be administered when the AAV vector comprising a nucleic acid molecule encoding OCT4, SOX2, and KLF4 and the AAV vector comprising a nucleic acid molecule encoding rtTA are administered. The antibiotic may be administered at least one day following administration of the AAV composition comprising the AAV vector comprising a nucleic acid molecule encoding OCT4, SOX2, and KLF4 and the AAV vector comprising a nucleic acid molecule encoding rtTA. The antibiotic may be administered 2 days, 3 days, 4 days, 5 days, or more than 5 days following administration of the AAV composition comprising the AAV vector comprising a nucleic acid molecule encoding OCT4, SOX2, and KLF4 and the AAV vector comprising a nucleic acid molecule encoding rtTA.

The disclosed methods of treating NAION include methods of administering to a subject in need thereof an effective amount of an AAV genome including one or more polynucleotide sequences expressing OCT4, SOX2, and KLF4 and an AAV genome comprising a nucleic acid sequence encoding transactivator 3 or transactivator 4.

### Methods of Preparing AAV-OSK Vectors

Provided herein are methods for recombinant preparation of an AAV. In some embodiments, the method comprises introducing one or more vectors as contemplated herein into a cell under conditions whereby the AAV is produced. The cell may include a population of cells. The population of cells may include any suitable cells as understood in the art, including for example HEK293 cells, HEK293T cells, COS cells, CHO cells, BHK cells, HeLa cells, and the like. The one or more vectors may be introduced into the cell using one or more suitable techniques including for example transfection, transduction, and/or infection. Exemplary methods for recombinant preparation of the AAV include transient transfection (e.g., with one or more transfer plasmids containing a first, and a second, and optionally a third vector as described herein), viral infection (e.g., with one or more recombinant helper viruses, such as a adenovirus, poxvirus (such as vaccinia virus), herpes virus (including HSV, cytomegalovirus, or baculovirus, containing a first, and a second, and optionally a third vector as described herein)), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more AAV capsid proteins as described herein, and with an AAV genome as described herein being delivered in the form of a plasmid or a recombinant helper virus). The first vector may include one or more nucleic acid sequences expressing OCT4, SOX2, and/or KLF4 encoded by one or more of SEQ ID NOs: 13, 14, 15, and 35. The second vector may include one or more nucleic acid sequences expressing transactivator 3, for example SEQ ID NO: 21 or 36. Alternatively, the second vector may include one or more nucleic acid sequences expressing transactivator 4, for example SEQ ID NO: 28 or 37.

Further provided herein are methods for generating an AAV comprising modifying a cell to express one or more plasmids. The one or more plasmids may include one or more AAV2 Rep-Cap plasmids, one or more helper plasmids, and one or more transfer plasmids. The one or more transfer plasmids may include a first transfer plasmid including one or more nucleic acids encoding one or more of: OCT4, SOX2, and KLF4; a second transfer plasmid including one or more nucleic acids encoding transactivator 3; or a transfer plasmid including one or more nucleic acids encoding one or more of: OCT4, SOX2, KLF4, and transactivator 3. The one or more transfer plasmids may also include a first transfer plasmid including one or more nucleic acids encoding one or more of: OCT4, SOX2, and KLF4; a second transfer plasmid including one or more nucleic acids encoding transactivator 4; or a transfer plasmid including one or more nucleic acids encoding one or more of: OCT4, SOX2, KLF4, and transactivator 4.

### EXAMPLES

### Example 1—Methods of Producing Vectors

The present study set out to develop a method for manufacturing batches of vectors and to assess the stability of the prepared batches.

AAV production protocol

### Materials

### Cells:

HEK293T cells were used to produce the vectors as described herein. The cells were grown in DMEM media (Invitrogen, cat. no. 11995073) containing 10% fetal bovine serum (FBS) (Invitrogen HI FBS, cat. no. 16140), penicillin/streptomycin (Invitrogen, cat. no. 15140-122), Glutamine (Invitrogen cat. no. 25030).

### Plasmids

The recombinant AAV2-TRE-OSK plasmid (shown in FIG. 2) was prepared using the following components: (1) a nucleic acid sequence encoding an AAV2 capsid protein or a fragment thereof, (2) a nucleic encoding a functional rep gene. (3) a recombinant AAV transfer vector comprising AAV2 inverted terminal repeats (SEQ ID NO: 16, SEQ ID NO: 32) flanking a transgene encoding OCT4, KLF4, and SOX2 (SEQ ID NO: 13) operably linked to an inducible TRE promoter (TRE3G, SEQ ID NO: 7), and (4) a helper vector with rAAV2 Rep-Cap proteins. Plasmids were obtained from Stratagene/Agilent (Stratagene cat no: 240071). The AAV2 Rep-Cap plasmid included an pAAV-RC plasmid (Stratagene cat. no. 240071). In some cases, AAV2 hybrid vectors e.g., AAV2/1 AAV2/2, AAV2/5, AAV2/6, AAV2/7, AAV2/8 and AAV2/9 with capsid proteins from AAV1, 2, 3, 5, 6, 7, 8, and 9 serotypes. The helper plasmid included a pHelper plasmid (Stratagene, cat. no. 240071) and carried adenovirus-derived genes for introducing helper functions. As shown in FIG. 2 the entire AAV2-TRE3G-OSK-SV40 pA vector is 7250 base pairs in length, and two inverted terminal repeats (ITRs) flank the OSK sequences.

The first expression vector encoding OCT4, SOX2, and KLF4 includes the nucleic acid sequence set forth in SEQ ID NO: 15. The recombinant AAV vector can include a nucleic acid encoding an inducing agent.

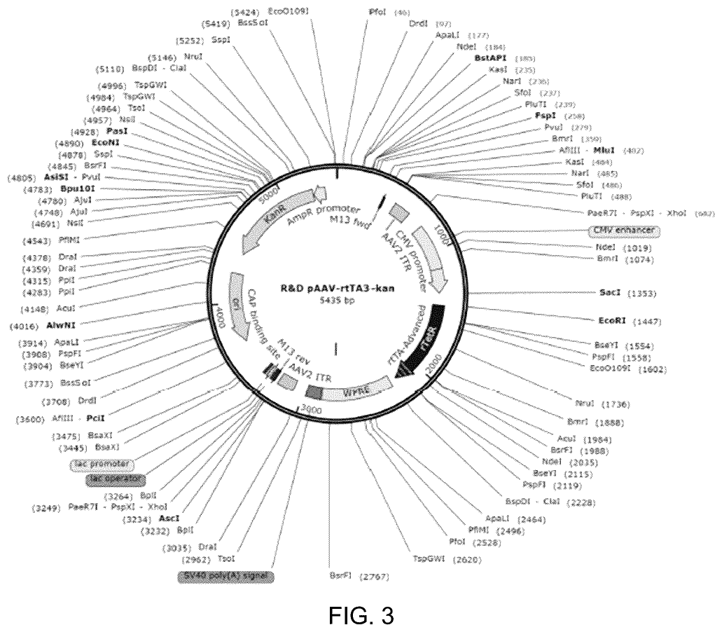

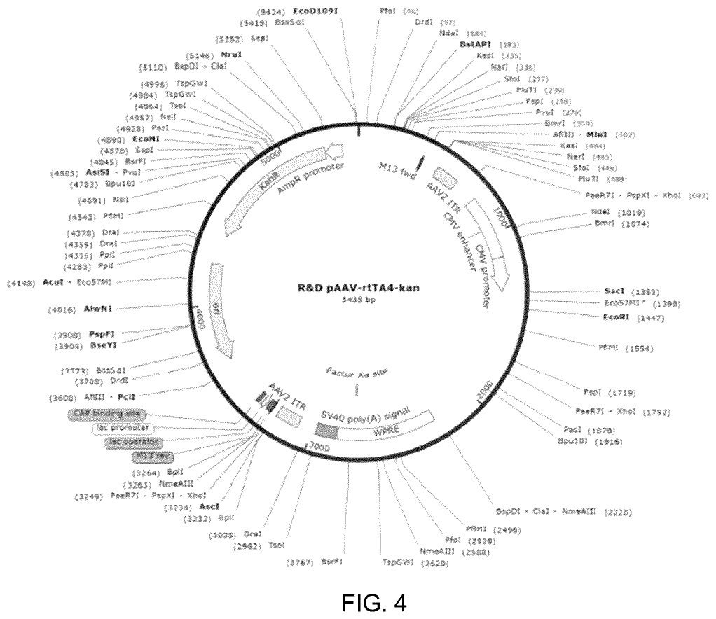

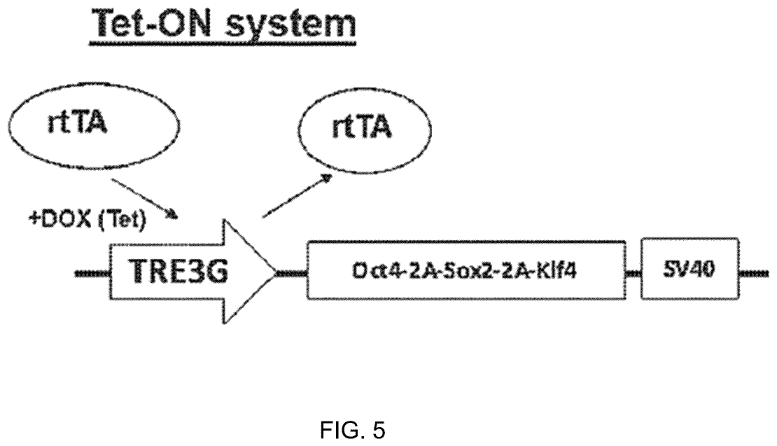

The recombinant pAAV2-CMV-rtTA, pAAV2-CMV-rtTA3(V16) (shown in FIG. 3), for the Tet-On plasmid was prepared using a similar approach to that described above but using a transfer plasmid with a CMV constitutive promoter (SEQ ID NO: 17) operably linked to rtTA3 (SEQ ID NO 19) inducing agent having 3 vp16 domains at the 3′ end. An alternative recombinant pAAV2-CMV-rtTA, pAAV2-CMV-rtTA4(V16) (shown in FIG. 4), may be prepared in the same way.

### Polyethyleneimine (PEI) Solution

PEI solution (1 μg/μl, Polysciences, cat. no. 23966-2) was prepared by dissolving PEI powder in H₂O that was heated to 80° C., cooled to room temperature, neutralized to pH 7.0, filter sterilized, aliquoted and stored at −20° ° C. The transfection efficiency was tested when each new batch was prepared.

### Methods

Vector production

On day 1, HEK293T cells were plated on 10 15-cm dishes before transfecting for 24 hours. Cells were seeded with 25 mL medium per 15-cm culture dish. Cells were split to a density of 70-90% (standard transfection density) ten times on 15 cm plates in order to provide a yield of 5E12 viral genomes (vg). Media was changed with 5% FBS to slow growth and reduce purification time one plate at a time in order to prevent cells from dying.

On day 2, 1 hour before transfection, medium was changed to 20 mL of freshly warmed medium. A DNA solution was prepared in a 50 mL FALCON™ tube. The amounts of DNA and reagents per dish are shown in the Table 2 below. Based on the size of inverted terminal repeat (ITR) plasmid, the amount of DNA was calculated. A tube of master mixture was prepared for 5 15-cm dishes. As shown in Table 2 below, all plasmids were diluted to 1 μg/μl in sterile H₂O.

A 10-mLsample of DMEM (without phenol red, Invitrogen cat. no. 31053-036) was prepared. A 785 μl aliquot of PEI solution was added, and the media was mixed. It was then incubated at room temperature for 20-30 minutes.

TABLE 2
Amounts of Reagents per Plate

| | | Amount of | Amount of | | |
|---|---|---|---|---|---|
| | Plasmid | Plasmid Per | Plasmid Per | [plasmid] | |
| Plasmid | size | Plate | 5 Plates | (μg/μl) | VOLUME |
| AAV2-RC | 7.3 kb | 15.2 μg | 76 μg | 1 μg/μl | 76 μl |
| HELPER | 11.6 kb | 24 μg | 120 μg | 1 μg/μl | 120 μl |
| ITR plasmid-Gene | 6.3 kb | 13.117808 μg | 65.58904 μg | 1 μg/μl | 65.5 μl |
| of Interest (e.g., | | | | | |
| OSK) | | | | | |

Next, 2 mL of DNA-PEI mixture was added to each of 10 15-cm dishes. The transfected cells were then incubated.

On Day 3, the medium was removed, and 25 mL of freshly warmed medium was added. Serum-containing medium was added for AAV2/2, AAV2/6. Serum-free medium was used for AAV2/1, AAV2/5, AAV2/7, AAV2/8 and AAV2/9.

On Day 5, cells were scraped with a cell scraper in their current medium and transferred to a 50 mLtube. The cell suspension was then spun at 1000 relative centrifugal force (rcf) for 5 minutes. The supernatant was then discarded.

### Transfection Procedures

All cells were combined into 1 50-mL tube, washed with PBS, and spun at 1000 rpm for 5 minutes. The supernatant was then discarded.

The cell pellet was resuspended in a volume of hypotonic buffer five times the volume of the packed cell volume (approximately 25 mL). It was then incubated on ice for 10 minutes. A 0.11% by volume amount of 10X restore buffer was then added and mixed by pipetting (generally a volume of 3.3 mL).

The nuclei were then spun down at 2000 rcf for 10 minutes, generating a nuclear pellet of about 1 mL per 10 15-cm dishes. The pellet was stored at −80° C. for further purification.

### Preparation of Solutions

5 M NaCl salt solution

A mass of 292.2 g NaCl was added to 200 of deionized (DI) water in a 2 L bottle. The mixture was shaken to mix, and poured into a large, graduated cylinder. DI water was then added up to 1 L and poured back into the 2 L bottle. A stir bar was added, and the volume was heated and stirred until dissolved. Alternatively, the volume was autoclaved. The solution was then cooled and sterile filtered through 0.2 μm filter.

### 40% PEG-8000, 2.5M NaCl (5× Stock Solution).

In a 1 L graduated cylinder, 400 g PEG-8000 and 500 mL 5 M NaCl were mixed. DI water was added to 1 L. This generally required about 100 ml of water. The solution was transferred to a 2 L flask and shaken to mix. A large stir bar was added, and the solution was heated overnight in a water bath at 55° C. The next day, the solution was sterile filtered with a 0.2 μm filter. This filtration step generally takes about 30 minutes.

Harvesting AAV from Media and Cells

The media and cells were harvested without use of trypsin. The media was collected with a pipetman and sterile filtered with a 0.2 μM filter. The cells were collected and spun down. Any remaining supernatant was added to the media sterile filtered with a 0.2 μm filter. The cells were harvested in one of several ways. Cells were collected with a cell scraper. Alternatively, cells were “blasted” with calcium- and magnesium-free PBS at a volume of 10 mL per 2 plates. A 40% solution of PEG 8000 adjusted to pH 7.4 was added to a final concentration of 12%. Approximately 25 mL per 100 mL of media/cells were used. The solution was stirred in a cold room for 1 hour and was either left to sit for 3 hours without spinning or was left overnight in cold room. The next day, the PEG mixture was spun at 3000×g for 20 minutes. The supernatant was discarded. The pellet was resuspended in less than about 7 mL of 1xPBS. The pellet was first suspended with about 5 mL and a volume of up to 2 mL was added once nearly suspended. A solution of benzonase at 1:10,000 was then added and the mixture was incubated for 45 minutes at 37ºC. Optionally, the mixture was spun down at 2415xg for 10 minutes at 4° C. The supernatant was transferred to new tube.

Concentrating AAV with Ultracentrifugation
Preparation of solutions

A 1 M MgCl₂ stock solution was prepared which was at 1000X and was used for making MK buffer. A 2.5 M stock solution of KCl was prepared which was a 1000x and was also used for preparing MK buffer.

A 1 M solution of NaCl/phosphate-buffered saline (PBS) MK buffer was prepared by dissolving 58.4 g of NaCl in 1 mL of 1 M MgCl₂ and 1 mL of 2.5 M KCl. Then, 1× PBS (Ca Mg Dulbecco's phosphate-buffered saline (DPBS), Gibco) was added to final volume of 1 L. The solution was sterilized by passing through a 0.22-μm filter and stored at 4° C. The solution was brought to final concentration of 1 M NaCl, 1 mM MgCl₂, and 2.5 mM KCl.

A 1× PBS-MK buffer was prepared by dissolving 1 mL of 1 M MgCl₂, and 1 mL 2.5 M KCl to 2×500 ml bottles of Ca- Mg-DPBS (Gibco). The solution was sterilized by passing through a 0.22-μm filter and stored at 4° C. The solution was brought to final concentrations of 1 mM MgCl₂ and 2.5 mM KCl.

A 0.001% Pluronic-F68 (formulation buffer) solution was prepared by adding 500 μL of sterile 1000X Pluronic F-68 (1% solution) to 500 mL of 1× DPBS (Gibco, TC stock). The solution was stored at 4° C. for up to one month, or aliquoted and stored at −80° ° C. for up to one year.

A 0.001% PLURONIC™-F68+5% sorbitol (freezing buffer) solution was prepared by adding 25 g of sorbitol to 500 μL of sterile 1000X PLURONIC™ F-68 (1% solution) to 500 mL of 1× DPBS (Gibco, TC stock). The solution was stored at 4° ° C. for up to one month, or aliquoted and stored at −80° C. for up to one year.

As shown in Table 3, 15% iodixanol solution was prepared by mixing 30 mL of 60% iodixanol and 90 mL of 1 M NaCl/PBS-MK buffer. A 25% iodixanol solution was prepared by mixing 112.5 mL of 60% iodixanol, 157.5 mL of 1x PBS-MK buffer, and 900 μL of phenol red. A 40% iodixanol solution was prepared by mixing 202.5 mL of 60% iodixanol and 67.5 mL of 1x PBS-MK buffer. A 60% iodixanol solution was prepared by mixing 150 mL of 60% iodixanol and 675 μL of phenol red.

TABLE 3
Iodixanol solutions

| % | mL 60% | mL 1M NaCl | mL PBS- | μL phenol |
|---|---|---|---|---|
| soln | iodixanol | PBS-MK | MK | red |
| 15 | 30 | 90 | 0 | 0 |
| 25 | 112.5 | 0 | 157.5 | 675 |
| 40 | 180 | 0 | 90 | 0 |
| 60 | 150 | 0 | 0 | 675 |

Note
all solutions were sterile-filtered with 0.2 μm filters.

In order to create an ultra-gradient, the benzonased supernatants were added to Beckman optiseal tubes. If volumes were unequal, 1 M NaCl, PBS-MK mix was added to make them equal, to a final volume of approximately 7 mL.

The tubes were filled from the bottom using 10 mL syringes and long hypodermic needles. For each solution the same syringe was reused but the needles were changed after every sample in order to prevent cross-contamination between AAV preparations. 5 mL was the minimum volume for any layer.

The tubes were balanced to 5-10 μg. The tubes were balanced in pairs. They were first sorted so that the most similar tubes were paired, and then PBS was added to the lighter tube in each pairing. PBS was added by touching the tip to the side of the tube to prevent droplets from disturbing the layers. Caps were added and tubes were loaded into the ultracentrifuge.

The tubes were then spun in an ultracentrifuge using a VTi50 rotor for 1 hour at max speed (50,000 rpm=242,000 xg). The fractions were collected from the ultracentrifuged tubes by piercing the bottom with an 18-gauge needle. The black stopper was removed at the top before piercing with the needle, otherwise air bubbles were created that disturb the layers. The majority of the 60% fraction was removed. The remaining part of the 60% layer was then collected along with the 40% fraction in a 50 mL tube. Sample collection was complete when the color changed significantly, or the solution was cloudy

### Separation of Fractions on a Protein Gel

Samples were denatured with 4x LDS with 2.5% β-mercaptoethanol at 70° ° C. for 10 minutes. In order to prepare 20 μL aliquots, 10 μL of sample was combined with 5 μL of H₂O, 5 μL of LDS with 2.5% β-mercaptoethanol. If necessary, extra running buffer (200 mL 10x Tris-Glycine SDS buffer, 1800 mL Millipure water) was prepared.

Samples were then loaded into a Tris Glycine gel. Either a 4-12% or 4-20% gradient gel was used. For AAV capsids, either is suitable.

The gel box was assembled, and the wells of the gel were loaded with 20 μL of sample per well. Gels were run at 225 V for 30-45 minutes, until the blue dye reached the bottom of the gel.

The gels were then stained with SYPRO™ red and imaged. First, a staining solution was prepared (7.5% acetic acid, SYPRO™ red is 5000x) by adding 10 μL of SYPRO™ red to 50 mL of stain solution. This was enough for 1 gel. Gels were stained for 1 hour while covered at room temperature on a slow rocker. The stain solution was then removed and acetic acid solution with no dye was added. The gels were incubated for 1 to 5 minutes in order to de-stain. The gels were images on a gel dock with EtBr settings (UV).

Good fractions were then combined and washed with 1xPBS with 0.001% F68. In some cases about 20 mL PBS+F68 was added to bring the final volume to just less than 30 mL in order to dilute the iodixanol which facilitates passing through the filter.

Samples were spun at 4700 g for 5 minutes. After everything flowed through, the samples were washed 3x more with 15 mL per wash in order to prepare a clear final solution. The samples were then aliquoted into labelled tubes (name of virus, payload, date) and stored at 4° ° C. for up to 1 week. For longer storage, the final elution was prepared with 1xPBS with 0.001% F68 and 5% sorbitol. Samples were frozen at −80° C.

Titering Virus with qPCR

Samples were prepared by aliquoting 12.5 μl Master Mix into tubes. Either fast advanced TAQMAN™ Master Mix from ThermoFisher or IDT Primetime Master Mix was used. To each tube, 0.0625 μl of primer 1, 0.0625 μl of primer 2, 0.125 μl of probe, 1 μl of virus, and 11.3 μl of H₂O was added for a total volume of 25 μl.

### Example 2—Method of Treating NAION in Non-Human Primates

This study set out to determine whether epigenetic reprogramming improves RGC function and restores visual function (pERG) in a nonhuman primate (NHP) model of NAION (Non-arteritic Anterior Ischemic Optic Neuropathy). The efficacy of the neuroprotective effect of the doxycycline responsive dual vector system AAV2-TRE-OSK (SEQ ID NO: 35)/AAV2-CMV-rtTA3V16 (SEQ ID NO: 36), in a photothrombotic experimental model of non-arteritic anterior ischemic optic neuropathy (NAION) was evaluated. The vector system was administered intravitreally (IVT) in African green monkeys induced by laser excitation of a systemically administered fluorophore at the optic nerve head (ONH).

### Methods

### Subject Recruitment

Monkeys with normal slit lamp and fundus exams, color fundus photographs (CFP), optical coherence tomography (OCT), confocal scanning laser ophthalmoscopy (cSLO), pattern electroretinograms (pERG), and pattern visual evoked potentials (pVEP) were recruited to the study. For baseline screening and all subsequent procedures, anesthesia was achieved with intramuscular ketamine (8 mg/kg) and xylazine (1.6 mg/kg) to effect, and pupil dilation with topical 10% phenylephrine and/or 1% cyclopentolate.

### Test Article

Subjects were administered a composition comprising a dual vector system including a AAV2-TRE-OSK vector and a AAV2-CMV-rtTA3V16 vector. The AAV2-TRE-OSK vector was prepared to a concentration of approximately 1.53×10¹² vg/mL and administered at a dose of 200 μL per dose to the subject. The AAV2-CMV-rtTA3V16 vector was prepared to a concentration of approximately 1.33×10¹³ vg/mL and administered at a dose of 20 μL per dose to the subject. Accordingly, the two vectors were administered in a ratio of 10:1 by volume (v/v) or a ratio of approximately 1:1 by viral genome ratio (vg/vg).

### Intravitreal Dosing

Eyes received a single intravitreal (IVT) injection of test article or vehicle in accordance with the treatment assignment (Table 4). For IVT dosing a drop of proparacaine hydrochloride 0.5% was applied to the eye followed by a lid speculum and 5% Betadine solution, and a rinse with sterile 0.9% saline. Injections were administered to the central vitreous using a 31-gauge 0.375 inch needle inserted inferotemporally at the level of the ora serrata ˜2.5 mm posterior to the limbus. Following injection, 1% topical atropine, a topical triple antibiotic neomycin, polymyxin, bacitracin ophthalmic ointment (or equivalent) was administered.

TABLE 4
Treatment Conditions

| | | | NAION | | | Concentration | | Dose | Test Article |
|---|---|---|---|---|---|---|---|---|---|
| Group | N | Eye | Induction | Treatment | Route | (vg/mL) | Volume | (vg/eye) | Required |
| 1A | 4 | OD | No | No treatment | — | — | — | — | — |
| | | | Laser | | | | | | |
| | | OS | Laser | Vehicle (PBS) | IVT | PBS | 220 | — | (220 μL + |
| | | | | 28 days pre- | | | | | 66 μL |
| | | | | NAION | | | | | overage) × |
| | | | | induction | | | | | 4 = 1144 μL |
| 1B | 4 | OD | No | No treatment | — | — | — | — | — |
| | | | Laser | | | | | | |
| | | OS | Laser | Vehicle | IVT | PBS | 220 | — | (220 μL + |
| | | | | 1 day post- | | | | | 66 μL |
| | | | | NAION | | | | | overage) × |
| | | | | induction | | | | | 4 = 1144 μL |
| 2 | 6 | OD | No | No treatment | — | — | — | — | — |
| | | | Laser | | | | | | |
| | | OS | Laser | Candidate #1 | IVT | OSK: | 220 | OSK: | (220 μL + |
| | | | | 28 days pre- | | 1.53 × 10¹² | | 3.06 × 10¹¹ | 66 μL |
| | | | | NAION | | rtTA3: | | rtTA3: | overage) × |
| | | | | induction | | 1.33 × 10¹³ | | 2.66 × 10¹¹ | 6 = 1716 μL |
| 3 | 6 | OD | No | No treatment | — | — | — | — | — |
| | | | Laser | | | | | | |
| | | OS | Laser | Candidate #1 | IVT | OSK: | 220 | OSK: | (220 μL + |
| | | | | 1 day post- | | 1.53 × 10¹² | | 3.06 × 10¹¹ | 66 μL |
| | | | | NAION | | rtTA3: | | rtTA3: | overage) × |
| | | | | induction | | 1.33 × 10¹³ | | 2.66 × 10¹¹ | 6 = 1716 μL |

### Doxycycline Dosing

Monkeys received oral doxycycline (5 mg/kg) in a food item (banana slice) starting one day prior to dosing and continuing through study terminus (Table 5).

TABLE 5
Dosing Schedule for Monkey Study

| | | | Study Day | | | | | | | | | | | | |
|---|---|---|---|---|---|---|---|---|---|---|---|---|---|---|---|
| | | | Base | | | | | | | | | | | | |
| Event | # | Eyes | line* | −21 | −19 | −7 | 0 | 1 | 3 | 7 | 14 | 28 | 35 | 42 | 49 |
| CBC EDTA | 24 | — | X | — | — | — | — | — | — | — | — | — | — | — | — |
| blood | | | | | | | | | | | | | | | |
| DNA | 20 | — | X | — | — | — | — | — | — | — | — | — | — | — | — |
| Methylation | | | | | | | | | | | | | | | |
| blood | | | | | | | | | | | | | | | |
| Serum | 20 | — | X | — | — | — | — | — | — | — | — | — | — | — | X |
| Photochrombosis | 20 | OS | — | — | — | — | X | — | — | X | — | — | — | — | — |
| IVT dosing | 10 | OS | — | Gp1A | — | — | — | — | — | — | — | — | — | — | — |
| Group 1A & 2 | | | | & 2 | | | | | | | | | | | |
| IVT dosing | 10 | OS | — | — | — | — | — | Gp1B | — | — | — | — | — | — | — |
| Group 1B & 3 | | | | | | | | &3 | | | | | | | |
| Vitreous Tap | 10 | OS | — | X§ | — | — | — | X§ | — | — | — | — | — | — | — |
| Doxycycline** | 20 | — | — | X | X | X | X | X | X | X | X | X | X | X | X |
| Methylprednisolone | 20 | — | — | Gp1A | — | Gp1A | X | — | — | X | X | X | X | X | X |
| | | | | & 2 | | & 2 | | | | | | | | | |
| Body weights | 20 | — | X | X | — | X | X | — | — | X | X | — | X | — | X |
| Detailed chemical | 20 | — | X | — | — | — | — | — | — | — | — | — | — | — | — |
| exams | | | | | | | | | | | | | | | |
| Tonometry | 20 | OU | X | — | — | X | — | — | — | X | X | — | X | — | X |
| Slit lamp exams | 20 | OU | X | — | Gp1a | X | — | — | Gp1B | X | X | — | X | — | X |
| | | | | | & 2 | | | | &3 | | | | | | |
| Fundus imaging | 20 | OU | X* | — | — | — | X | X | — | X | X | — | X | — | X |
| Spectralis cSLO | 20 | OU | X | — | — | — | X | X | — | X | X | — | X | — | X |
| Spectralis OCT | 20 | OU | X | — | — | — | X | X | — | X | X | — | X | — | X |
| Pattern ERG*** | 20 | OU | X | — | — | X | — | — | — | X | X | X | X | — | X |
| Pattern VEP*** | 20 | OU | X | — | — | — | — | — | — | X | X | X | X | — | X |
| ffERG*** | 20 | OU | — | — | — | — | — | — | — | — | — | — | — | X | X |
| Enucleation | 20 | OU | — | — | — | — | — | — | — | — | — | — | — | — | X |

X = event for all treatment groups; Gp1A = event for Group 1A; Gp1B = event for Group 1B; Gp2 = event for Group 2; Gp3 = event for Group 3.
*Baseline prescreen assessments will include evaluation of up to 24 monkeys to meet the targeted number of study recruits.
**Daily oral doxycycline dosing will begin at day −21 for all animals and continue until day 42.
***Exams will be completed within +−4 days.
§Vitreous tap done immediately prior to dosing

### Immunosuppression

Monkeys received methylprednisolone (40 mg IM) weekly starting on Day 0 (Table 5). If inflammation exceeding 2+anterior and/or vitreous cell was observed on subsequent exam subconjunctival dexamethasone (100 mL of 40 mg/mL) was administered, with repeat dosing guided by clinical observations. If inflammation did not respond to dexamethasone, methylprednisolone was increased to 80 mg IM weekly. Administered steroids were applied consistently across all treatment animals.

### NAION Induction

Monkeys were divided by exam and weight criteria into two cohorts. After achieving mydriasis and placement of a saphenous vein catheter, rose bengal (2.5 mg/kg; 0.1 ml/kg of 25 mg/ml) were administered intravenously (IV), followed 25 seconds later by initiation of laser spot application to the ONH. Laser spots were applied with an Iridex Oculight TX 532 nm laser using a 0.9x contact laser lens. Four spots were placed in the four quadrants of the ONH, each of pulse duration 6 seconds, spot size 500 μm, power 100 mW. The first spot was applied in the superatemporal aspect of the ONH, the second in the inferotemporal, the third in the inferonasal, and the fourth in the superanasal. Branched retinal veins and arteries were avoided in the placement of each spot. Post-laser CFP imaging was performed immediately to document response to laser photothrombosis procedure.

### Tonometry

Intraocular pressure (IOP) was measured oculus uterque (OU) or in both eyes using a TonoVet tonometer set to the dog (d) calibration setting. Three measures were taken from each eye at each ophthalmic examination time point (Table 8) and the mean IOP was defined.

### Vitreous Humor Collection

At designated time points (Table 5) vitreous humor (˜170 μL) was collected OU with a 1.0 mL syringe with a 27-gauge needle introduced into the mid-vitreous by temporal scleral puncture at the level of the ora serrata. Vitreous humor aliquots were transferred to pre-labeled cryotubes, flash frozen, and stored and shipped for analysis.

Full Field Electroretinography (ffERG)

At designated time points (Table 5), slit lamp biomicroscopy and retinoscopy were performed in both eyes (OU). employing an LKC UTAS full-field ERG apparatus and emWin software (Version 9.8.0). Immediately after sedation and topical instillation of 10% phenylephrine and 1% cyclopentolate, eyes were dark-adapted for 30 minutes. Additional mydriatic were administered prior to stimulus exposure. ffERGs were performed OD then OS (or OS then OD in random order). After placement of a DTL electrode (or Burian-Allen contact lens electrode), a subdermal reference electrode at the ipsilateral lateral canthus, and ground electrode in the upper limb for unilateral sequential testing a scotopic rod-specific response was elicited by a dim white flash in a LED Ganzfeld stimulator bowl. Mixed rod and cone responses were obtained using standard bright white flashes under scotopic conditions. To evaluate the photopic function of cone photoreceptors, monkeys were light-adapted to ambient room light for 10 minutes, after which a strobe white-flash stimulus was presented to the dilated eye using maximum flash intensity. The following ISCEV stimulus standards for toxicology studies were applied:
Scotopic 0.158 cd-s m2 (-12 dB) stimulus (rod-driven response of on bipolar cells measured, b-wave)
Scotopic 2.51 cd-s m2 (0 dB) stimulus (rod and cone-driven response of both photoreceptors, a-wave, and on bipolar cells, b-wave)
Photopic 2.51 cd-s m2 (0 dB) stimulus (cone driven response of both photoreceptors, a-wave, and on and off bipolar cells, b-wave)
Photopic 30 Hz flicker stimulus at 2.51 cd-s m2 (0 dB) stimulus (cone driven response)

Stimulus induction was denoted by a marker. The time integrated luminance of the stimulus and the background was recorded in absolute values. Monkeys underwent scotopic exams before photopic exams and underwent stimulus exposure order of increasing stimulus strength for a given adaptation. Single stimulus exposures preceded flicker stimulus exposure to avoid bleaching and loss of adaptation. Waveforms were analyzed for a- and b-wave amplitudes and latency. The amplitude of the b-wave was measured from the a-wave trough to the peak of the b-wave or, if no a-wave was present, from the pre-stimulus baseline to the peak of the b-wave. The amplitude of the a-wave was measured from the pre-stimulus baseline to the peak of the a-wave. A nonhuman primate ophthalmic scoring system was employed to assess ocular pathology changes in response to NAION induction and treatment intervention with a summary score derived from exam components. Incidence of papilledema and flame hemorrhage was also evaluated.

### Imaging

Color anterior segment and fundus photography was performed using a Topcon TRC-50EX retinal camera with Canon 6D digital imaging hardware and New Vision Fundus Image Analysis System software.

Optical Coherence Tomography (OCT) and Confocal Scanning Laser Ophthalmoscopy (cSLO)

At designated time points (Table 5), cSLO and OCT were performed using a Heidelberg Spectralis HRA OCT with HEYEX image capture and analysis software. cSLO infrared (IR) and autofluorescence (AF) images were obtained at 30° field of view centered on the fovea and the follow-up imaging function referencing the baseline images. An overall OCT volume scan of the optic nerve and entire macula was performed by posterior pole. Images were qualitatively assessed with quantitative analysis of calculated retinal thickness. For the macula centered posterior pole scan, ganglion cell layer (GCL) and retinal nerve fiber layer (RNFL) thickness maps were generated, and the thickness data exported to an electronic spreadsheet. Peripapillary retinal nerve fiber layer thickness (pRNFL) thickness was determined in each quadrant (superior, inferior, temporal, nasal). HEYEX raw data files were generated for additional analyses.

### Pattern ERG and VEP

At designated time points (Table 5), pattern ERGs (pERGs) and pattern VEP (pVEP) recordings were performed OU by positioning the monkeys with a DTL electrode and an additional active electrode placed in the inion in the midline at the posterior of the skull, with a ground electrode placed in the arm. A VERIS retinal projecting stimulator was used to project an alternating black and white checkerboard pattern at 2 Hz with a luminance of 100 cd/m2 at a contrast of 80% over a field of 45% generated by a VERIS multifocal ERG instrument. Grid size was varied in logarithmic steps, recordings at each grid size repeated twice and latencies and amplitudes determined. pVEP amplitude and pERG N95 amplitude were determined.

### Clinical Observations

General wellbeing was assessed twice daily by cage side observation beginning one week prior to dosing and extending to study terminus.

### Detailed Clinical Examination

Detailed clinical observations and physical exams were performed at designated time points (Table 5). Respiratory rate, heart rate, blood pressure, auscultation and integrity of the integument were also assessed. Body temperature was determined using a digital rectal thermometer.

### Body Weights

Body weights were collected at ophthalmic exam intervals (Table 5).

CBC with Differential

Blood (0.5 mL) was transferred directly to K2EDTA lavender top vacutainer tubes (Greiner MiniCollect EDTA tubes REF #450475) and maintained on ice until CBC with differentials analysis on a Hemavet analyzer.

### DNA Methylation Blood

Blood was transferred directly to K2EDTA anticoagulated 0.5 mL microcentrifuge tube. Samples will then be lysed, and DNA isolated on QIAMP MiniElute columns per the manufacturer's instructions (Qiagen). DNA samples were analyzed for DNA methylation measures of biologic age.

### Serum

At designated time points (Table 5), blood (3 ml) was transferred directly to serum separator tubes (red top) and allowed to sit at room temperature for 30 to 60 minutes prior to centrifugation at 3000 rpm for 10 minutes at 4° C. Two serum aliquots (˜0.5 mL×2) as available were carefully transferred to labeled 1.8 mL cryotubes and stored and shipped below −70° C. to a designated lab for nAb analysis.

### Enucleation and Eye Processing

At study terminus (Table 5), after confirmation of the quality of in-life imaging and electrophysiology, monkeys were sedated with intramuscular ketamine (8 mg/kg) and xylazine (1.6 mg/kg) to effect, animals were then euthanized with sodium pentobarbital (100 mg/kg IV) to effect. Globes (OU) were enucleated after placing a suture marker at the 12 o'clock position. Globes with attached optic nerve were trimmed of excess tissue and placed in Davidson's fixative at room temperature for 24 hours with injection of ˜300 μl of Davidson's fixative into the globe. The globes were transferred to phosphate buffered saline (PBS) with 0.05% sodium azide and stored and shipped in a container maintained at 4° ° C. to the designated histology lab for histological and immunohistochemical processing and analysis by a board-certified veterinary pathologist. Additional quantitative scoring of histology was conducted, guided by qualitative findings.

pERG Results

A NAION-like injury was induced on Day 0 in all NHPs (African Green monkeys; N=20) by iv rose bengal followed by laser treatment of the OS eye ONH. Pre-treated NHPs (n=6) received on Day −28 an intravitreal (IVT) injection of OSK (doxycycline-inducible AAV2-OSK; 1:1 ratio of AAV2-TRE-OSK+AAV2-CMV-rtTA3), or vehicle (n=4) into the OS eye. Post-treatment NHPs received on Day+1 an IVT injection of OSK (n=6), or vehicle (n=4) into the OS eye. All NHPs received daily oral doxycycline throughout the experiment. Both eyes were examined at baseline then at weekly intervals until Day+35 post-laser treatment (corresponding to Day 42) for multiple imaging and functional measures of retinal ganglion cells (RGCs).

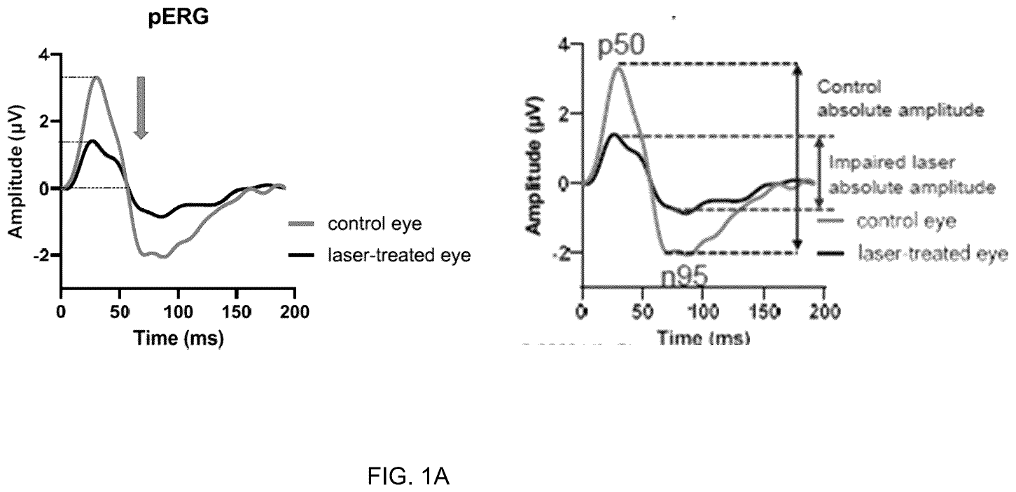

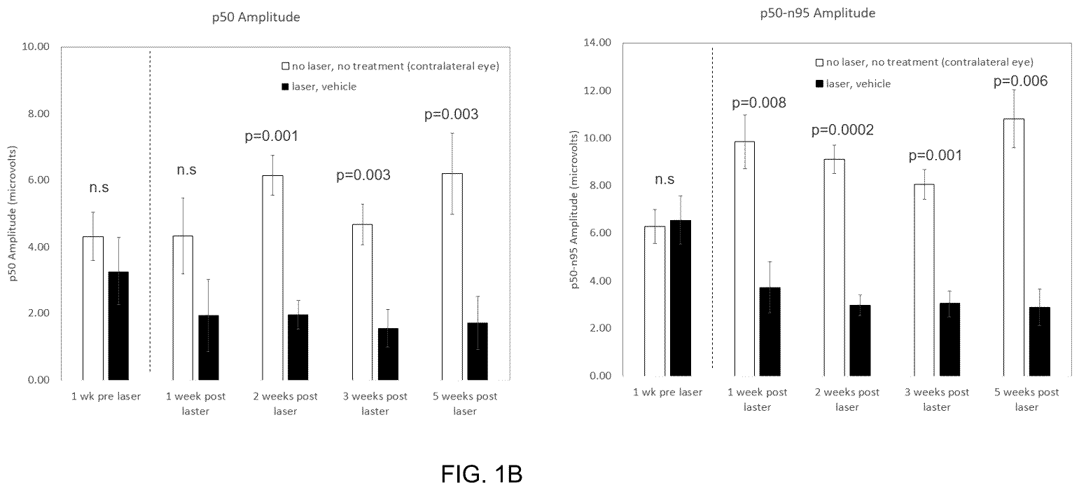

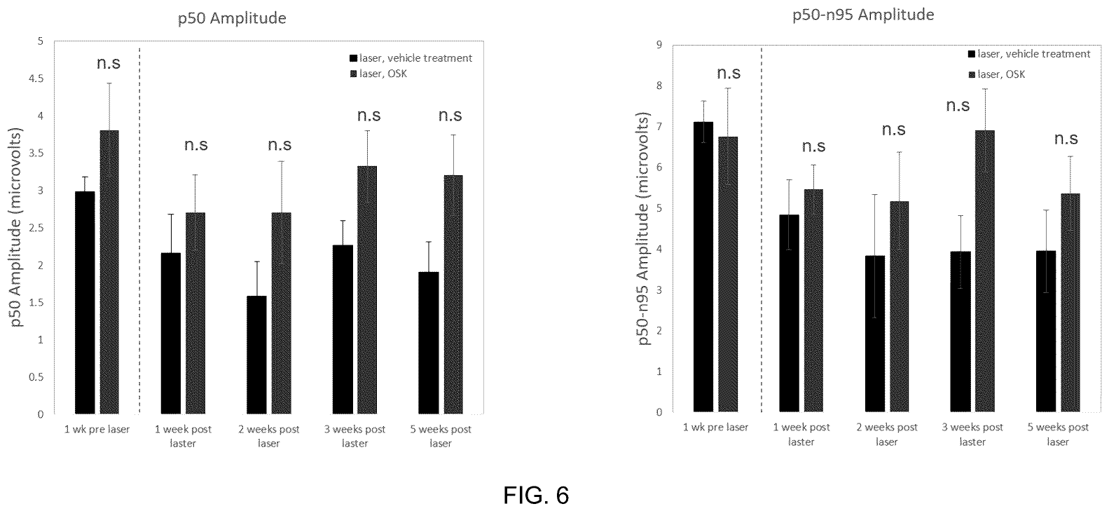

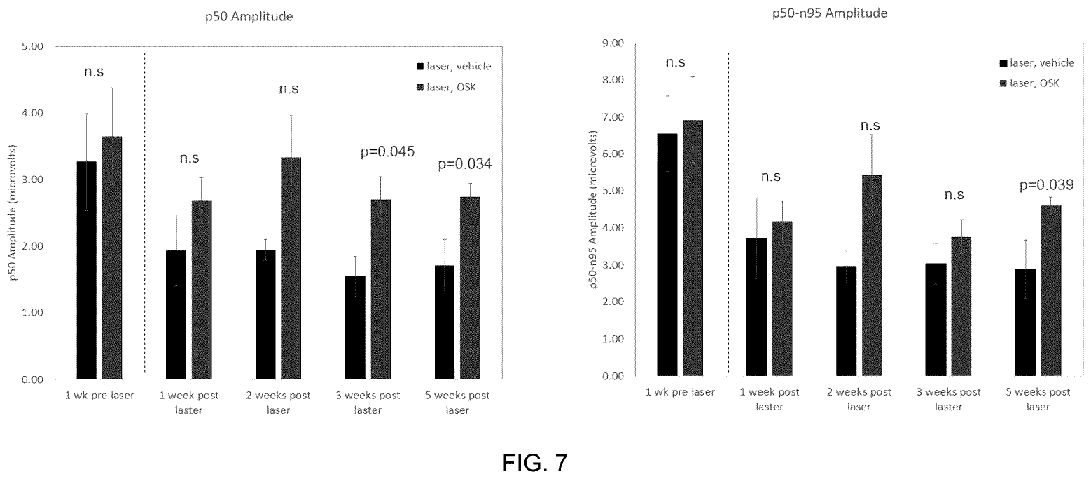

Consistent with NAION pERG deficits in humans, the laser induced a NAION-like injury in vehicle treated OS eyes with a significant loss in pERG amplitude relative to OD (untreated) eyes (FIGS. 1A and 1B). Pretreatment with OSK in OS eyes increased pERG amplitude and partially reversed the NAION-like pERG deficits compared with vehicle-treated control eyes at the endpoint (p50-n95 amplitude: 5.36+0.91 vs 3.95+1.01, n.s., 5 weeks post-laser treatment; see FIG. 6). Post-treatment with OSK showed a significant recovery of pERG function by 5 weeks post-laser treatment (p50-n95 amplitude: 4.60+0.24 vs 2.89+0.79, P=0.039) (FIG. 7).

### Results on Rescuing Axon Damage

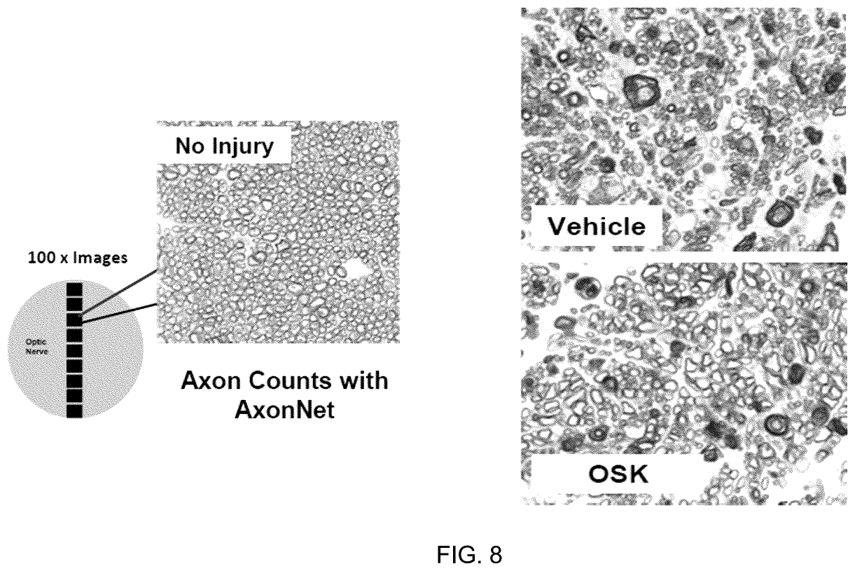

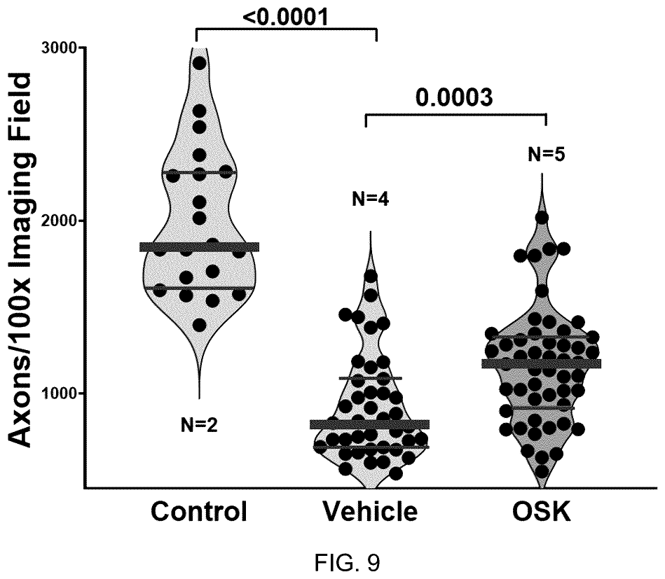

Optic Nerve Myelin-Specific (PPD) Axon Stain was used to show the function of OSK expression to rescue axon damages subjected to induction of the NAION injury. As shown in FIG. 8, compared to vehicle treatment, post-expression of OSK rescued axon damages and increased axon numbers (counted with AxonNet; for a review of AxonNet, see Ehrlich and Rivera, AxonNet: A self-supervised Deep Neural Network for Intravoxel Structure Estimation from DW-MRI. ArXiv abs/2103.11006 (2021)). The enhancement of axon survival by OSK, compared to vehicle treatment, after laser-induced damage is further shown in FIG. 9.

As a summary for the NHP NAION study described herein, treatment with OSK improves RGC function, consistent with potential improvement in vision. In addition, treatment with OSK increases the number of healthy axons in the optic nerve, consistent with the potential for regeneration in the animals.

### Equivalents and Incorporation by Reference

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

While various specific embodiments have been illustrated and described, it will be appreciated that various changes can be made without departing from the spirit and scope of the disclosure.

TABLE 6
AAV2-TRE-OSK Vector Sequences

| SEQ ID | | |
|---|---|---|
| NO: | Component | Sequence |
| 1 | Human | ATGGCGGGACACCTGGCTTCGGATTTCGCCTTCTCGCCCCCTC |
| | OCT4 | CAGGTGGTGGAGGTGATGGGCCAGGGGGGCCGGAGCCGGGCTG |
| | nucleic acid | GGTTGATCCTCGGACCTGGCTAAGCTTCCAAGGCCCTCCTGGA |
| | sequence | GGGCCAGGAATCGGGCCGGGGGTTGGGCCAGGCTCTGAGGTGT |
| | | GGGGGATTCCCCCATGCCCCCCGCCGTATGAGTTCTGTGGGGG |
| | | GATGGCGTACTGTGGGCCCCAGGTTGGAGTGGGGCTAGTGCCC |
| | | CAAGGCGGCTTGGAGACCTCTCAGCCTGAGGGCGAAGCAGGAG |
| | | TCGGGGTGGAGAGCAACTCCGATGGGGCCTCCCCGGAGCCCTG |
| | | CACCGTCACCCCTGGTGCCGTGAAGCTGGAGAAGGAGAAGCTG |
| | | GAGCAAAACCCGGAGGAGTCCCAGGACATCAAAGCTCTGCAGA |
| | | AAGAACTCGAGCAATTTGCCAAGCTCCTGAAGCAGAAGAGGAT |
| | | CACCCTGGGATATACACAGGCCGATGTGGGGCTCACCCTGGGG |
| | | GTTCTATTTGGGAAGGTATTCAGCCAAACGACCATCTGCCGCT |
| | | TTGAGGCTCTGCAGCTTAGCTTCAAGAACATGTGTAAGCTGCG |
| | | GCCCTTGCTGCAGAAGTGGGTGGAGGAAGCTGACAACAATGAA |
| | | AATCTTCAGGAGATATGCAAAGCAGAAACCCTCGTGCAGGCCC |
| | | GAAAGAGAAAGCGAACCAGTATCGAGAACCGAGTGAGAGGCAA |
| | | CCTGGAGAATTTGTTCCTGCAGTGCCCGAAACCCACACTGCAG |
| | | CAGATCAGCCACATCGCCCAGCAGCTTGGGCTCGAGAAGGATG |
| | | TGGTCCGAGTGTGGTTCTGTAACCGGCGCCAGAAGGGCAAGCG |
| | | ATCAAGCAGCGACTATGCACAACGAGAGGATTTTGAGGCTGCT |
| | | GGGTCTCCTTTCTCAGGGGGACCAGTGTCCTTTCCTCTGGCCC |
| | | CAGGGCCCCATTTTGGTACCCCAGGCTATGGGAGCCCTCACTT |
| | | CACTGCACTGTACTCCTCGGTCCCTTTCCCTGAGGGGGAAGCC |
| | | TTTCCCCCTGTCTCTGTCACCACTCTGGGCTCTCCCATGCATT |
| | | CAAAC |
| 2 | Human | MAGHLASDFAFSPPPGGGGDGPGGPEPGWVDPRTWLSFQGPPG |
| | OCT4 amino | GPGIGPGVGPGSEVWGIPPCPPPYEFCGGMAYCGPQVGVGLVP |
| | acid | QGGLETSQPEGEAGVGVESNSDGASPEPCTVTPGAVKLEKEKL |
| | sequence | EQNPEESQDIKALQKELEQFAKLLKQKRITLGYTQADVGLTLG |
| | | VLFGKVFSQTTICRFEALQLSFKNMCKLRPLLQKWVEEADNNE |
| | | NLQEICKAETLVQARKRKRTSIENRVRGNLENLFLQCPKPTLQ |
| | | QISHIAQQLGLEKDVVRVWFCNRRQKGKRSSSDYAQREDFEAA |
| | | GSPFSGGPVSFPLAPGPHFGTPGYGSPHFTALYSSVPFPEGEA |
| | | FPPVSVTTLGSPMHSN |
| 3 | Human | ATGTACAACATGATGGAGACGGAGCTGAAGCCGCCGGGCCCGC |
| | SOX2 | AGCAAACTTCGGGGGGCGGCGGCGGCAACTCCACCGCGGCGGC |
| | nucleic acid | GGCCGGCGGCAACCAGAAAAACAGCCCGGACCGCGTCAAGCGG |
| | sequence | CCCATGAATGCCTTCATGGTGTGGTCCCGCGGGCAGCGGCGCA |
| | | AGATGGCCCAGGAGAACCCCAAGATGCACAACTCGGAGATCAG |
| | | CAAGCGCCTGGGCGCCGAGTGGAAACTTTTGTCGGAGACGGAG |
| | | AAGCGGCCGTTCATCGACGAGGCTAAGCGGCTGCGAGCGCTGC |
| | | ACATGAAGGAGCACCCGGATTATAAATACCGGCCCCGGCGGAA |
| | | AACCAAGACGCTCATGAAGAAGGATAAGTACACGCTGCCCGGC |
| | | GGGCTGCTGGCCCCCGGCGGCAATAGCATGGCGAGCGGGGTCG |
| | | GGGTGGGCGCCGGCCTGGGCGCGGGCGTGAACCAGCGCATGGA |
| | | CAGTTACGCGCACATGAACGGCTGGAGCAACGGCAGCTACAGC |
| | | ATGATGCAGGACCAGCTGGGCTACCCGCAGCACCCGGGCCTCA |
| | | ATGCGCACGGCGCAGCGCAGATGCAGCCCATGCACCGCTACGA |
| | | CGTGAGCGCCCTGCAGTACAACTCCATGACCAGCTCGCAGACC |
| | | TACATGAACGGCTCGCCCACCTACAGCATGTCCTACTCGCAGC |
| | | AGGGCACCCCTGGCATGGCTCTTGGCTCCATGGGTTCGGTGGT |
| | | CAAGTCCGAGGCCAGCTCCAGCCCCCCTGTGGTTACCTCTTCC |
| | | TCCCACTCCAGGGCGCCCTGCCAGGCCGGGGACCTCCGGGACA |
| | | TGATCAGCATGTATCTCCCCGGCGCCGAGGTGCCGGAACCCGC |
| | | CGCCCCCAGCAGACTTCACATGTCCCAGCACTACCAGAGCGGC |
| | | CCGGTGCCCGGCACGGCCATTAACGGCACACTGCCCCTCTCAC |
| | | ACATG |
| 4 | Human | MYNMMETELKPPGPQQTSGGGGGNSTAAAAGGNQKNSPDRVKR |
| | SOX2 amino | PMNAFMVWSRGQRRKMAQENPKMHNSEISKRLGAEWKLLSETE |
| | acid | KRPFIDEAKRLRALHMKEHPDYKYRPRRKTKTLMKKDKYTLPG |
| | sequence | GLLAPGGNSMASGVGVGAGLGAGVNQRMDSYAHMNGWSNGSYS |
| | | MMQDQLGYPQHPGLNAHGAAQMQPMHRYDVSALQYNSMTSSQT |
| | | YMNGSPTYSMSYSQQGTPGMALGSMGSVVKSEASSSPPVVTSS |
| | | SHSRAPCQAGDLRDMISMYLPGAEVPEPAAPSRLHMSQHYQSG |
| | | PVPGTAINGTLPLSHM |
| 5 | Human | ATGGCTGTCAGCGACGCGCTGCTCCCATCTTTCTCCACGTTCG |
| | KLF4 nucleic | CGTCTGGCCCGGCGGGAAGGGAGAAGACACTGCGTCAAGCAGG |
| | acid | TGCCCCGAATAACCGCTGGCGGGAGGAGCTCTCCCACATGAAG |
| | sequence | CGACTTCCCCCAGTGCTTCCCGGCCGCCCCTATGACCTGGCGG |
| | | CGGCGACCGTGGCCACAGACCTGGAGAGCGGCGGAGCCGGTGC |
| | | GGCTTGCGGCGGTAGCAACCTGGCGCCCCTACCTCGGAGAGAG |
| | | ACCGAGGAGTTCAACGATCTCCTGGACCTGGACTTTATTCTCT |
| | | CCAATTCGCTGACCCATCCTCCGGAGTCAGTGGCCGCCACCGT |
| | | GTCCTCGTCAGCGTCAGCCTCCTCTTCGTCGTCGCCGTCGAGC |
| | | AGCGGCCCTGCCAGCGCGCCCTCCACCTGCAGCTTCACCTATC |
| | | CGATCCGGGCCGGGAACGACCCGGGCGTGGCGCCGGGCGGCAC |
| | | GGGCGGAGGCCTCCTCTATGGCAGGGAGTCCGCTCCCCCTCCG |
| | | ACGGCTCCCTTCAACCTGGCGGACATCAACGACGTGAGCCCCT |
| | | CGGGCGGCTTCGTGGCCGAGCTCCTGCGGCCAGAATTGGACCC |
| | | GGTGTACATTCCGCCGCAGCAGCCGCAGCCGCCAGGTGGCGGG |
| | | CTGATGGGCAAGTTCGTGCTGAAGGCGTCGCTGAGCGCCCCTG |
| | | GCAGCGAGTACGGCAGCCCGTCGGTCATCAGCGTCAGCAAAGG |
| | | CAGCCCTGACGGCAGCCACCCGGTGGTGGTGGCGCCCTACAAC |
| | | GGCGGGCCGCCGCGCACGTGCCCCAAGATCAAGCAGGAGGCGG |
| | | TCTCTTCGTGCACCCACTTGGGCGCTGGACCCCCTCTCAGCAA |
| | | TGGCCACCGGCCGGCTGCACACGACTTCCCCCTGGGGCGGCAG |
| | | CTCCCCAGCAGGACTACCCCGACCCTGGGTCTTGAGGAAGTGC |
| | | TGAGCAGCAGGGACTGTCACCCTGCCCTGCCGCTTCCTCCCGG |
| | | CTTCCATCCCCACCCGGGGCCCAATTACCCATCCTTCCTGCCC |
| | | GATCAGATGCAGCCGCAAGTCCCGCCGCTCCATTACCAAGAGC |
| | | TCATGCCACCCGGTTCCTGCATGCCAGAGGAGCCCAAGCCAAA |
| | | GAGGGGAAGACGATCGTGGCCCCGGAAAAGGACCGCCACCCAC |
| | | ACTTGTGATTACGCGGGCTGCGGCAAAACCTACACAAAGAGTT |
| | | CCCATCTCAAGGCACACCTGCGAACCCACACAGGTGAGAAACC |
| | | TTACCACTGTGACTGGGACGGCTGTGGATGGAAATTCGCCCGC |
| | | TCAGATGAACTGACCAGGCACTACCGTAAACACACGGGGCACC |
| | | GCCCGTTCCAGTGCCAAAAATGCGACCGAGCATTTTCCAGGTC |
| | | GGACCACCTCGCCTTACACATGAAGAGGCATTTT |
| 6 | Human | MAVSDALLPSFSTFASGPAGREKTLRQAGAPNNRWREELSHMK |
| | KLF4 amino | RLPPVLPGRPYDLAAATVATDLESGGAGAACGGSNLAPLPRRE |
| | acid | TEEFNDLLDLDFILSNSLTHPPESVAATVSSSASASSSSSPSS |
| | sequence | SGPASAPSTCSFTYPIRAGNDPGVAPGGTGGGLLYGRESAPPP |
| | | TAPFNLADINDVSPSGGFVAELLRPELDPVYIPPQQPQPPGGG |
| | | LMGKFVLKASLSAPGSEYGSPSVISVSKGSPDGSHPVVVAPYN |
| | | GGPPRTCPKIKQEAVSSCTHLGAGPPLSNGHRPAAHDFPLGRQ |
| | | LPSRTTPTLGLEEVLSSRDCHPALPLPPGFHPHPGPNYPSFLP |
| | | DQMQPQVPPLHYQELMPPGSCMPEEPKPKRGRRSWPRKRTATH |
| | | TCDYAGCGKTYTKSSHLKAHLRTHTGEKPYHCDWDGCGWKFAR |
| | | SDELTRHYRKHTGHRPFQCQKCDRAFSRSDHLALHMKRHF |
| 7 | TRE3G | TTTACTCCCTATCAGTGATAGAGAACGTATGAAGAGTTTACTC |
| | (TRE | CCTATCAGTGATAGAGAACGTATGCAGACTTTACTCCCTATCA |
| | promoter) | GTGATAGAGAACGTATAAGGAGTTTACTCCCTATCAGTGATAG |
| | | AGAACGTATGACCAGTTTACTCCCTATCAGTGATAGAGAACGT |
| | | ATCTACAGTTTACTCCCTATCAGTGATAGAGAACGTATATCCA |
| | | GTTTACTCCCTATCAGTGATAGAGAACGTATAAGCTTTAGGCG |
| | | TGTACGGTGGGCGCCTATAAAAGCAGAGCTCGTTTAGTGAACC |
| | | GTCAGATCGCCTGGAGCAATTCCACAACACTTTTGTCTTATAC |
| | | CAACTTTCCGTACCACTTCCTACCCTCGTAAA |
| 8 | P2A peptide | GCCACGAACTTCTCTCTGTTAAAGCAAGCAGGAGATGTTGAAG |
| | from porcine | AAAACCCCGGGCCT |
| | teschovirus-1 | |
| | polyprotein | |
| 9 | P2A amino | ATNFSLLKQAGDVEENPGP |
| | acid | |
| 10 | T2A peptide | GAGGGCAGGGGAAGTCTTCTAACATGCGGGGACGTGGAGGAAA |
| | from Thosea | ATCCCGGCCCA |
| | asigna virus | |
| | capsid | |
| | protein | |
| 11 | T2A | EGRGSLLTCGDVEENPGP |
| | cleavage | |
| | sequence | |
| | amino acid | |
| 12 | SV40 | AACTTGTTTATTGCAGCTTATAATGGTTACAAATAAAGCAATA |
| | | GCATCACAAATTTCACAAATAAAGCATTTTTTTCACTGCATTC |
| | | TAGTTGTGGTTTGTCCAAACTCATCAATGTATCTTATCATGTC |
| | | TGGATC |
| 13 | OCT4-2A- | CTATGCGGCATCAGAGCAGATTGTACTGAGAGTGCACCATATG |
| | SOX2-2A- | CGGTGTGAAATACCGCACAGATGCGTAAGGAGAAAATACCGCA |
| | KLF4 | TCAGGCGCCATTCGCCATTCAGGCTGCGCAACTGTTGGGAAGG |
| | (Whole insert | GCGATCGGTGCGGGCCTCTTCGCTATTACGCCAGCTGGCGAAA |
| | sequence | GGGGGATGTGCTGCAAGGCGATTAAGTTGGGTAACGCCAGGGT |
| | including Tet | TTTCCCAGTCACGACGTTGTAAAACGACGGCCAGTGAATTGAC |
| | operators and | GCGTATTGGGATCCATGATTACGCCAGATTTAATTAAGGCCTT |
| | SV40, minus | AATTAGGCTGCGCGCTCGCTCGCTCACTGAGGCCGCCCGGGCA |
| | ITRs) | AAGCCCGGGCGTCGGGCGACCTTTGGTCGCCCGGCCTCAGTGA |
| | | GCGAGCGAGCGCGCAGAGAGGGAGTGGCCAACTCCATCACTAG |
| | | GGGTTCCTTGTAGTTAATGATTAACCCGCCATGCTACTTATCT |
| | | ACGTAGCCATGCTCTAGGAAGATCGGAATTCTTTACTCCCTAT |
| | | CAGTGATAGAGAACGTATGAAGAGTTTACTCCCTATCAGTGAT |
| | | AGAGAACGTATGCAGACTTTACTCCCTATCAGTGATAGAGAAC |
| | | GTATAAGGAGTTTACTCCCTATCAGTGATAGAGAACGTATGAC |
| | | CAGTTTACTCCCTATCAGTGATAGAGAACGTATCTACAGTTTA |
| | | CTCCCTATCAGTGATAGAGAACGTATATCCAGTTTACTCCCTA |
| | | TCAGTGATAGAGAACGTATAAGCTTTAGGCGTGTACGGTGGGC |
| | | GCCTATAAAAGCAGAGCTCGTTTAGTGAACCGTCAGATCGCCT |
| | | GGAGCAATTCCACAACACTTTTGTCTTATACCAACTTTCCGTA |
| | | CCACTTCCTACCCTCGTAAAGCGGCCGCGCCACCATGGCGGGA |
| | | CACCTGGCTTCGGATTTCGCCTTCTCGCCCCCTCCAGGTGGTG |
| | | GAGGTGATGGGCCAGGGGGGCCGGAGCCGGGCTGGGTTGATCC |
| | | TCGGACCTGGCTAAGCTTCCAAGGCCCTCCTGGAGGGCCAGGA |
| | | ATCGGGCCGGGGGTTGGGCCAGGCTCTGAGGTGTGGGGGATTC |
| | | CCCCATGCCCCCCGCCGTATGAGTTCTGTGGGGGGATGGCGTA |
| | | CTGTGGGCCCCAGGTTGGAGTGGGGCTAGTGCCCCAAGGCGGC |
| | | TTGGAGACCTCTCAGCCTGAGGGCGAAGCAGGAGTCGGGGTGG |
| | | AGAGCAACTCCGATGGGGCCTCCCCGGAGCCCTGCACCGTCAC |
| | | CCCTGGTGCCGTGAAGCTGGAGAAGGAGAAGCTGGAGCAAAAC |
| | | CCGGAGGAGTCCCAGGACATCAAAGCTCTGCAGAAAGAACTCG |
| | | AGCAATTTGCCAAGCTCCTGAAGCAGAAGAGGATCACCCTGGG |
| | | ATATACACAGGCCGATGTGGGGCTCACCCTGGGGGTTCTATTT |
| | | GGGAAGGTATTCAGCCAAACGACCATCTGCCGCTTTGAGGCTC |
| | | TGCAGCTTAGCTTCAAGAACATGTGTAAGCTGCGGCCCTTGCT |
| | | GCAGAAGTGGGTGGAGGAAGCTGACAACAATGAAAATCTTCAG |
| | | GAGATATGCAAAGCAGAAACCCTCGTGCAGGCCCGAAAGAGAA |
| | | AGCGAACCAGTATCGAGAACCGAGTGAGAGGCAACCTGGAGAA |
| | | TTTGTTCCTGCAGTGCCCGAAACCCACACTGCAGCAGATCAGC |
| | | CACATCGCCCAGCAGCTTGGGCTCGAGAAGGATGTGGTCCGAG |
| | | TGTGGTTCTGTAACCGGCGCCAGAAGGGCAAGCGATCAAGCAG |
| | | CGACTATGCACAACGAGAGGATTTTGAGGCTGCTGGGTCTCCT |
| | | TTCTCAGGGGGACCAGTGTCCTTTCCTCTGGCCCCAGGGCCCC |
| | | ATTTTGGTACCCCAGGCTATGGGAGCCCTCACTTCACTGCACT |
| | | GTACTCCTCGGTCCCTTTCCCTGAGGGGGAAGCCTTTCCCCCT |
| | | GTCTCTGTCACCACTCTGGGCTCTCCCATGCATTCAAACGCTA |
| | | GCGGCAGCGGCGCCACGAACTTCTCTCTGTTAAAGCAAGCAGG |
| | | AGATGTTGAAGAAAACCCCGGGCCTGCATGCATGTACAACATG |
| | | ATGGAGACGGAGCTGAAGCCGCCGGGCCCGCAGCAAACTTCGG |
| | | GGGGCGGCGGCGGCAACTCCACCGCGGCGGCGGCCGGCGGCAA |
| | | CCAGAAAAACAGCCCGGACCGCGTCAAGCGGCCCATGAATGCC |
| | | TTCATGGTGTGGTCCCGCGGGCAGCGGCGCAAGATGGCCCAGG |
| | | AGAACCCCAAGATGCACAACTCGGAGATCAGCAAGCGCCTGGG |
| | | CGCCGAGTGGAAACTTTTGTCGGAGACGGAGAAGCGGCCGTTC |
| | | ATCGACGAGGCTAAGCGGCTGCGAGCGCTGCACATGAAGGAGC |
| | | ACCCGGATTATAAATACCGGCCCCGGCGGAAAACCAAGACGCT |
| | | CATGAAGAAGGATAAGTACACGCTGCCCGGCGGGCTGCTGGCC |
| | | CCCGGCGGCAATAGCATGGCGAGCGGGGTCGGGGTGGGCGCCG |
| | | GCCTGGGCGCGGGCGTGAACCAGCGCATGGACAGTTACGCGCA |
| | | CATGAACGGCTGGAGCAACGGCAGCTACAGCATGATGCAGGAC |
| | | CAGCTGGGCTACCCGCAGCACCCGGGCCTCAATGCGCACGGCG |
| | | CAGCGCAGATGCAGCCCATGCACCGCTACGACGTGAGCGCCCT |
| | | GCAGTACAACTCCATGACCAGCTCGCAGACCTACATGAACGGC |
| | | TCGCCCACCTACAGCATGTCCTACTCGCAGCAGGGCACCCCTG |
| | | GCATGGCTCTTGGCTCCATGGGTTCGGTGGTCAAGTCCGAGGC |
| | | CAGCTCCAGCCCCCCTGTGGTTACCTCTTCCTCCCACTCCAGG |
| | | GCGCCCTGCCAGGCCGGGGACCTCCGGGACATGATCAGCATGT |
| | | ATCTCCCCGGCGCCGAGGTGCCGGAACCCGCCGCCCCCAGCAG |
| | | ACTTCACATGTCCCAGCACTACCAGAGCGGCCCGGTGCCCGGC |
| | | ACGGCCATTAACGGCACACTGCCCCTCTCACACATGGCATGCG |
| | | GCTCCGGCGAGGGCAGGGGAAGTCTTCTAACATGCGGGGACGT |
| | | GGAGGAAAATCCCGGCCCACTCGAGATGGCTGTCAGCGACGCG |
| | | CTGCTCCCATCTTTCTCCACGTTCGCGTCTGGCCCGGCGGGAA |
| | | GGGAGAAGACACTGCGTCAAGCAGGTGCCCCGAATAACCGCTG |
| | | GCGGGAGGAGCTCTCCCACATGAAGCGACTTCCCCCAGTGCTT |
| | | CCCGGCCGCCCCTATGACCTGGCGGCGGCGACCGTGGCCACAG |
| | | ACCTGGAGAGCGGCGGAGCCGGTGCGGCTTGCGGCGGTAGCAA |
| | | CCTGGCGCCCCTACCTCGGAGAGAGACCGAGGAGTTCAACGAT |
| | | CTCCTGGACCTGGACTTTATTCTCTCCAATTCGCTGACCCATC |
| | | CTCCGGAGTCAGTGGCCGCCACCGTGTCCTCGTCAGCGTCAGC |
| | | CTCCTCTTCGTCGTCGCCGTCGAGCAGCGGCCCTGCCAGCGCG |
| | | CCCTCCACCTGCAGCTTCACCTATCCGATCCGGGCCGGGAACG |
| | | ACCCGGGCGTGGCGCCGGGCGGCACGGGCGGAGGCCTCCTCTA |
| | | TGGCAGGGAGTCCGCTCCCCCTCCGACGGCTCCCTTCAACCTG |
| | | GCGGACATCAACGACGTGAGCCCCTCGGGCGGCTTCGTGGCCG |
| | | AGCTCCTGCGGCCAGAATTGGACCCGGTGTACATTCCGCCGCA |
| | | GCAGCCGCAGCCGCCAGGTGGCGGGCTGATGGGCAAGTTCGTG |
| | | CTGAAGGCGTCGCTGAGCGCCCCTGGCAGCGAGTACGGCAGCC |
| | | CGTCGGTCATCAGCGTCAGCAAAGGCAGCCCTGACGGCAGCCA |
| | | CCCGGTGGTGGTGGCGCCCTACAACGGCGGGCCGCCGCGCACG |
| | | TGCCCCAAGATCAAGCAGGAGGCGGTCTCTTCGTGCACCCACT |
| | | TGGGCGCTGGACCCCCTCTCAGCAATGGCCACCGGCCGGCTGC |
| | | ACACGACTTCCCCCTGGGGCGGCAGCTCCCCAGCAGGACTACC |
| | | CCGACCCTGGGTCTTGAGGAAGTGCTGAGCAGCAGGGACTGTC |
| | | ACCCTGCCCTGCCGCTTCCTCCCGGCTTCCATCCCCACCCGGG |
| | | GCCCAATTACCCATCCTTCCTGCCCGATCAGATGCAGCCGCAA |
| | | GTCCCGCCGCTCCATTACCAAGAGCTCATGCCACCCGGTTCCT |
| | | GCATGCCAGAGGAGCCCAAGCCAAAGAGGGGAAGACGATCGTG |
| | | GCCCCGGAAAAGGACCGCCACCCACACTTGTGATTACGCGGGC |
| | | TGCGGCAAAACCTACACAAAGAGTTCCCATCTCAAGGCACACC |
| | | TGCGAACCCACACAGGTGAGAAACCTTACCACTGTGACTGGGA |
| | | CGGCTGTGGATGGAAATTCGCCCGCTCAGATGAACTGACCAGG |
| | | CACTACCGTAAACACACGGGGCACCGCCCGTTCCAGTGCCAAA |
| | | AATGCGACCGAGCATTTTCCAGGTCGGACCACCTCGCCTTACA |
| | | CATGAAGAGGCATTTTTAAATGACTAGTGCGCGCAGCGGCCGA |
| | | CCATGGCCCAACTTGTTTATTGCAGCTTATAATGGTTACAAAT |
| | | AAAGCAATAGCATCACAAATTTCACAAATAAAGCATTTTTTTC |
| | | ACTGCATTCTAGTTGTGGTTTGTCCAAACTCATCAATGTATCT |
| | | TATCATGTCTGGATCTCGGTACCGGATCCAAATTCCCGATAAG |
| | | GATCTTCCTAGAGCATGGCTACGTAGATAAGTAGCATGGCGGG |
| | | TTAATCATTAACTACAAGGAACCCCTAGTGATGGAGTTGGCCA |
| | | CTCCCTCTCTGCGCGCTCGCTCGCTCACTGAGGCCGGGCGACC |
| | | AAAGGTCGCCCGACGCCCGGGCTTTGCCCGGGCGGCCTCAGTG |
| | | AGCGAGCGAGCGCGCAGCCTTAATTAACCTAATTCATCCCAAT |
| | | GGCGCGCCGAGCTTGGCTCGAGCATGGTCAT |
| 14 | OCT4-2A- | ATGGCGGGACACCTGGCTTCGGATTTCGCCTTCTCGCCCCCTC |
| | SOX2-2A- | CAGGTGGTGGAGGTGATGGGCCAGGGGGGCCGGAGCCGGGCTG |
| | KLF4 | GGTTGATCCTCGGACCTGGCTAAGCTTCCAAGGCCCTCCTGGA |
| | (Whole insert | GGGCCAGGAATCGGGCCGGGGGTTGGGCCAGGCTCTGAGGTGT |
| | sequence, | GGGGGATTCCCCCATGCCCCCCGCCGTATGAGTTCTGTGGGGG |
| | does not | GATGGCGTACTGTGGGCCCCAGGTTGGAGTGGGGCTAGTGCCC |
| | include SV40 | CAAGGCGGCTTGGAGACCTCTCAGCCTGAGGGCGAAGCAGGAG |
| | or Tet | TCGGGGTGGAGAGCAACTCCGATGGGGCCTCCCCGGAGCCCTG |
| | Operators) | CACCGTCACCCCTGGTGCCGTGAAGCTGGAGAAGGAGAAGCTG |
| | | GAGCAAAACCCGGAGGAGTCCCAGGACATCAAAGCTCTGCAGA |
| | | AAGAACTCGAGCAATTTGCCAAGCTCCTGAAGCAGAAGAGGAT |
| | | CACCCTGGGATATACACAGGCCGATGTGGGGCTCACCCTGGGG |
| | | GTTCTATTTGGGAAGGTATTCAGCCAAACGACCATCTGCCGCT |
| | | TTGAGGCTCTGCAGCTTAGCTTCAAGAACATGTGTAAGCTGCG |
| | | GCCCTTGCTGCAGAAGTGGGTGGAGGAAGCTGACAACAATGAA |
| | | AATCTTCAGGAGATATGCAAAGCAGAAACCCTCGTGCAGGCCC |
| | | GAAAGAGAAAGCGAACCAGTATCGAGAACCGAGTGAGAGGCAA |
| | | CCTGGAGAATTTGTTCCTGCAGTGCCCGAAACCCACACTGCAG |
| | | CAGATCAGCCACATCGCCCAGCAGCTTGGGCTCGAGAAGGATG |
| | | TGGTCCGAGTGTGGTTCTGTAACCGGCGCCAGAAGGGCAAGCG |
| | | ATCAAGCAGCGACTATGCACAACGAGAGGATTTTGAGGCTGCT |
| | | GGGTCTCCTTTCTCAGGGGGACCAGTGTCCTTTCCTCTGGCCC |
| | | CAGGGCCCCATTTTGGTACCCCAGGCTATGGGAGCCCTCACTT |
| | | CACTGCACTGTACTCCTCGGTCCCTTTCCCTGAGGGGGAAGCC |
| | | TTTCCCCCTGTCTCTGTCACCACTCTGGGCTCTCCCATGCATT |
| | | CAAACGCTAGCGGCAGCGGCGCCACGAACTTCTCTCTGTTAAA |
| | | GCAAGCAGGAGATGTTGAAGAAAACCCCGGGCCTGCATGCATG |
| | | TACAACATGATGGAGACGGAGCTGAAGCCGCCGGGCCCGCAGC |
| | | AAACTTCGGGGGGCGGCGGCGGCAACTCCACCGCGGCGGCGGC |
| | | CGGCGGCAACCAGAAAAACAGCCCGGACCGCGTCAAGCGGCCC |
| | | ATGAATGCCTTCATGGTGTGGTCCCGCGGGCAGCGGCGCAAGA |
| | | TGGCCCAGGAGAACCCCAAGATGCACAACTCGGAGATCAGCAA |
| | | GCGCCTGGGCGCCGAGTGGAAACTTTTGTCGGAGACGGAGAAG |
| | | CGGCCGTTCATCGACGAGGCTAAGCGGCTGCGAGCGCTGCACA |
| | | TGAAGGAGCACCCGGATTATAAATACCGGCCCCGGCGGAAAAC |
| | | CAAGACGCTCATGAAGAAGGATAAGTACACGCTGCCCGGCGGG |
| | | CTGCTGGCCCCCGGCGGCAATAGCATGGCGAGCGGGGTCGGGG |
| | | TGGGCGCCGGCCTGGGCGCGGGCGTGAACCAGCGCATGGACAG |
| | | TTACGCGCACATGAACGGCTGGAGCAACGGCAGCTACAGCATG |
| | | ATGCAGGACCAGCTGGGCTACCCGCAGCACCCGGGCCTCAATG |
| | | CGCACGGCGCAGCGCAGATGCAGCCCATGCACCGCTACGACGT |
| | | GAGCGCCCTGCAGTACAACTCCATGACCAGCTCGCAGACCTAC |
| | | ATGAACGGCTCGCCCACCTACAGCATGTCCTACTCGCAGCAGG |
| | | GCACCCCTGGCATGGCTCTTGGCTCCATGGGTTCGGTGGTCAA |
| | | GTCCGAGGCCAGCTCCAGCCCCCCTGTGGTTACCTCTTCCTCC |
| | | CACTCCAGGGCGCCCTGCCAGGCCGGGGACCTCCGGGACATGA |
| | | TCAGCATGTATCTCCCCGGCGCCGAGGTGCCGGAACCCGCCGC |
| | | CCCCAGCAGACTTCACATGTCCCAGCACTACCAGAGCGGCCCG |
| | | GTGCCCGGCACGGCCATTAACGGCACACTGCCCCTCTCACACA |
| | | TGGCATGCGGCTCCGGCGAGGGCAGGGGAAGTCTTCTAACATG |
| | | CGGGGACGTGGAGGAAAATCCCGGCCCACTCGAGATGGCTGTC |
| | | AGCGACGCGCTGCTCCCATCTTTCTCCACGTTCGCGTCTGGCC |
| | | CGGCGGGAAGGGAGAAGACACTGCGTCAAGCAGGTGCCCCGAA |
| | | TAACCGCTGGCGGGAGGAGCTCTCCCACATGAAGCGACTTCCC |
| | | CCAGTGCTTCCCGGCCGCCCCTATGACCTGGCGGCGGCGACCG |
| | | TGGCCACAGACCTGGAGAGCGGCGGAGCCGGTGCGGCTTGCGG |
| | | CGGTAGCAACCTGGCGCCCCTACCTCGGAGAGAGACCGAGGAG |
| | | TTCAACGATCTCCTGGACCTGGACTTTATTCTCTCCAATTCGC |
| | | TGACCCATCCTCCGGAGTCAGTGGCCGCCACCGTGTCCTCGTC |
| | | AGCGTCAGCCTCCTCTTCGTCGTCGCCGTCGAGCAGCGGCCCT |
| | | GCCAGCGCGCCCTCCACCTGCAGCTTCACCTATCCGATCCGGG |
| | | CCGGGAACGACCCGGGCGTGGCGCCGGGCGGCACGGGCGGAGG |
| | | CCTCCTCTATGGCAGGGAGTCCGCTCCCCCTCCGACGGCTCCC |
| | | TTCAACCTGGCGGACATCAACGACGTGAGCCCCTCGGGCGGCT |
| | | TCGTGGCCGAGCTCCTGCGGCCAGAATTGGACCCGGTGTACAT |
| | | TCCGCCGCAGCAGCCGCAGCCGCCAGGTGGCGGGCTGATGGGC |
| | | AAGTTCGTGCTGAAGGCGTCGCTGAGCGCCCCTGGCAGCGAGT |
| | | ACGGCAGCCCGTCGGTCATCAGCGTCAGCAAAGGCAGCCCTGA |
| | | CGGCAGCCACCCGGTGGTGGTGGCGCCCTACAACGGCGGGCCG |
| | | CCGCGCACGTGCCCCAAGATCAAGCAGGAGGCGGTCTCTTCGT |
| | | GCACCCACTTGGGCGCTGGACCCCCTCTCAGCAATGGCCACCG |
| | | GCCGGCTGCACACGACTTCCCCCTGGGGCGGCAGCTCCCCAGC |
| | | AGGACTACCCCGACCCTGGGTCTTGAGGAAGTGCTGAGCAGCA |
| | | GGGACTGTCACCCTGCCCTGCCGCTTCCTCCCGGCTTCCATCC |
| | | CCACCCGGGGCCCAATTACCCATCCTTCCTGCCCGATCAGATG |
| | | CAGCCGCAAGTCCCGCCGCTCCATTACCAAGAGCTCATGCCAC |
| | | CCGGTTCCTGCATGCCAGAGGAGCCCAAGCCAAAGAGGGGAAG |
| | | ACGATCGTGGCCCCGGAAAAGGACCGCCACCCACACTTGTGAT |
| | | TACGCGGGCTGCGGCAAAACCTACACAAAGAGTTCCCATCTCA |
| | | AGGCACACCTGCGAACCCACACAGGTGAGAAACCTTACCACTG |
| | | TGACTGGGACGGCTGTGGATGGAAATTCGCCCGCTCAGATGAA |
| | | CTGACCAGGCACTACCGTAAACACACGGGGCACCGCCCGTTCC |
| | | AGTGCCAAAAATGCGACCGAGCATTTTCCAGGTCGGACCACCT |
| | | CGCCTTACACATGAAGAGGCATTTT |
| 15 | pAAV2- | TCGCGCGTTTCGGTGATGACGGTGAAAACCTCTGACACATGCA |
| | TRE3G- | GCTCCCGGAGACTGTCACAGCTTGTCTGTAAGCGGATGCCGGG |
| | OSK-SV40 | AGCAGACAAGCCCGTCAGGGCGCGTCAGCGGGTGTTGGCGGGT |
| | (full plasmid | GTCGGGGCTGGCTTAACTATGCGGCATCAGAGCAGATTGTACT |
| | sequence, | GAGAGTGCACCATATGCGGTGTGAAATACCGCACAGATGCGTA |
| | including | AGGAGAAAATACCGCATCAGGCGCCATTCGCCATTCAGGCTGC |
| | selection | GCAACTGTTGGGAAGGGCGATCGGTGCGGGCCTCTTCGCTATT |
| | casettes) | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAATTGACGCGTATTGGGATCCATGATTACGCCAG |
| | | ATTTAATTAAGGCCTTAATTAGGCTGCGCGCTCGCTCGCTCAC |
| | | TGAGGCCGCCCGGGCAAAGCCCGGGCGTCGGGCGACCTTTGGT |
| | | CGCCCGGCCTCAGTGAGCGAGCGAGCGCGCAGAGAGGGAGTGG |
| | | CCAACTCCATCACTAGGGGTTCCTTGTAGTTAATGATTAACCC |
| | | GCCATGCTACTTATCTACGTAGCCATGCTCTAGGAAGATCGGA |
| | | ATTCTTTACTCCCTATCAGTGATAGAGAACGTATGAAGAGTTT |
| | | ACTCCCTATCAGTGATAGAGAACGTATGCAGACTTTACTCCCT |
| | | ATCAGTGATAGAGAACGTATAAGGAGTTTACTCCCTATCAGTG |
| | | ATAGAGAACGTATGACCAGTTTACTCCCTATCAGTGATAGAGA |
| | | ACGTATCTACAGTTTACTCCCTATCAGTGATAGAGAACGTATA |
| | | TCCAGTTTACTCCCTATCAGTGATAGAGAACGTATAAGCTTTA |
| | | GGCGTGTACGGTGGGCGCCTATAAAAGCAGAGCTCGTTTAGTG |
| | | AACCGTCAGATCGCCTGGAGCAATTCCACAACACTTTTGTCTT |
| | | ATACCAACTTTCCGTACCACTTCCTACCCTCGTAAAGCGGCCG |
| | | CGCCACCATGGCGGGACACCTGGCTTCGGATTTCGCCTTCTCG |
| | | CCCCCTCCAGGTGGTGGAGGTGATGGGCCAGGGGGGCCGGAGC |
| | | CGGGCTGGGTTGATCCTCGGACCTGGCTAAGCTTCCAAGGCCC |
| | | TCCTGGAGGGCCAGGAATCGGGCCGGGGGTTGGGCCAGGCTCT |
| | | GAGGTGTGGGGGATTCCCCCATGCCCCCCGCCGTATGAGTTCT |
| | | GTGGGGGGATGGCGTACTGTGGGCCCCAGGTTGGAGTGGGGCT |
| | | AGTGCCCCAAGGCGGCTTGGAGACCTCTCAGCCTGAGGGCGAA |
| | | GCAGGAGTCGGGGTGGAGAGCAACTCCGATGGGGCCTCCCCGG |
| | | AGCCCTGCACCGTCACCCCTGGTGCCGTGAAGCTGGAGAAGGA |
| | | GAAGCTGGAGCAAAACCCGGAGGAGTCCCAGGACATCAAAGCT |
| | | CTGCAGAAAGAACTCGAGCAATTTGCCAAGCTCCTGAAGCAGA |
| | | AGAGGATCACCCTGGGATATACACAGGCCGATGTGGGGCTCAC |
| | | CCTGGGGGTTCTATTTGGGAAGGTATTCAGCCAAACGACCATC |
| | | TGCCGCTTTGAGGCTCTGCAGCTTAGCTTCAAGAACATGTGTA |
| | | AGCTGCGGCCCTTGCTGCAGAAGTGGGTGGAGGAAGCTGACAA |
| | | CAATGAAAATCTTCAGGAGATATGCAAAGCAGAAACCCTCGTG |
| | | CAGGCCCGAAAGAGAAAGCGAACCAGTATCGAGAACCGAGTGA |
| | | GAGGCAACCTGGAGAATTTGTTCCTGCAGTGCCCGAAACCCAC |
| | | ACTGCAGCAGATCAGCCACATCGCCCAGCAGCTTGGGCTCGAG |
| | | AAGGATGTGGTCCGAGTGTGGTTCTGTAACCGGCGCCAGAAGG |
| | | GCAAGCGATCAAGCAGCGACTATGCACAACGAGAGGATTTTGA |
| | | GGCTGCTGGGTCTCCTTTCTCAGGGGGACCAGTGTCCTTTCCT |
| | | CTGGCCCCAGGGCCCCATTTTGGTACCCCAGGCTATGGGAGCC |
| | | CTCACTTCACTGCACTGTACTCCTCGGTCCCTTTCCCTGAGGG |
| | | GGAAGCCTTTCCCCCTGTCTCTGTCACCACTCTGGGCTCTCCC |
| | | ATGCATTCAAACGCTAGCGGCAGCGGCGCCACGAACTTCTCTC |
| | | TGTTAAAGCAAGCAGGAGATGTTGAAGAAAACCCCGGGCCTGC |
| | | ATGCATGTACAACATGATGGAGACGGAGCTGAAGCCGCCGGGC |
| | | CCGCAGCAAACTTCGGGGGGCGGCGGCGGCAACTCCACCGCGG |
| | | CGGCGGCCGGCGGCAACCAGAAAAACAGCCCGGACCGCGTCAA |
| | | GCGGCCCATGAATGCCTTCATGGTGTGGTCCCGCGGGCAGCGG |
| | | CGCAAGATGGCCCAGGAGAACCCCAAGATGCACAACTCGGAGA |
| | | TCAGCAAGCGCCTGGGCGCCGAGTGGAAACTTTTGTCGGAGAC |
| | | GGAGAAGCGGCCGTTCATCGACGAGGCTAAGCGGCTGCGAGCG |
| | | CTGCACATGAAGGAGCACCCGGATTATAAATACCGGCCCCGGC |
| | | GGAAAACCAAGACGCTCATGAAGAAGGATAAGTACACGCTGCC |
| | | CGGCGGGCTGCTGGCCCCCGGCGGCAATAGCATGGCGAGCGGG |
| | | GTCGGGGTGGGCGCCGGCCTGGGCGCGGGCGTGAACCAGCGCA |
| | | TGGACAGTTACGCGCACATGAACGGCTGGAGCAACGGCAGCTA |
| | | CAGCATGATGCAGGACCAGCTGGGCTACCCGCAGCACCCGGGC |
| | | CTCAATGCGCACGGCGCAGCGCAGATGCAGCCCATGCACCGCT |
| | | ACGACGTGAGCGCCCTGCAGTACAACTCCATGACCAGCTCGCA |
| | | GACCTACATGAACGGCTCGCCCACCTACAGCATGTCCTACTCG |
| | | CAGCAGGGCACCCCTGGCATGGCTCTTGGCTCCATGGGTTCGG |
| | | TGGTCAAGTCCGAGGCCAGCTCCAGCCCCCCTGTGGTTACCTC |
| | | TTCCTCCCACTCCAGGGCGCCCTGCCAGGCCGGGGACCTCCGG |
| | | GACATGATCAGCATGTATCTCCCCGGCGCCGAGGTGCCGGAAC |
| | | CCGCCGCCCCCAGCAGACTTCACATGTCCCAGCACTACCAGAG |
| | | CGGCCCGGTGCCCGGCACGGCCATTAACGGCACACTGCCCCTC |
| | | TCACACATGGCATGCGGCTCCGGCGAGGGCAGGGGAAGTCTTC |
| | | TAACATGCGGGGACGTGGAGGAAAATCCCGGCCCACTCGAGAT |
| | | GGCTGTCAGCGACGCGCTGCTCCCATCTTTCTCCACGTTCGCG |
| | | TCTGGCCCGGCGGGAAGGGAGAAGACACTGCGTCAAGCAGGTG |
| | | CCCCGAATAACCGCTGGCGGGAGGAGCTCTCCCACATGAAGCG |
| | | ACTTCCCCCAGTGCTTCCCGGCCGCCCCTATGACCTGGCGGCG |
| | | GCGACCGTGGCCACAGACCTGGAGAGCGGCGGAGCCGGTGCGG |
| | | CTTGCGGCGGTAGCAACCTGGCGCCCCTACCTCGGAGAGAGAC |
| | | CGAGGAGTTCAACGATCTCCTGGACCTGGACTTTATTCTCTCC |
| | | AATTCGCTGACCCATCCTCCGGAGTCAGTGGCCGCCACCGTGT |
| | | CCTCGTCAGCGTCAGCCTCCTCTTCGTCGTCGCCGTCGAGCAG |
| | | CGGCCCTGCCAGCGCGCCCTCCACCTGCAGCTTCACCTATCCG |
| | | ATCCGGGCCGGGAACGACCCGGGCGTGGCGCCGGGCGGCACGG |
| | | GCGGAGGCCTCCTCTATGGCAGGGAGTCCGCTCCCCCTCCGAC |
| | | GGCTCCCTTCAACCTGGCGGACATCAACGACGTGAGCCCCTCG |
| | | GGCGGCTTCGTGGCCGAGCTCCTGCGGCCAGAATTGGACCCGG |
| | | TGTACATTCCGCCGCAGCAGCCGCAGCCGCCAGGTGGCGGGCT |
| | | GATGGGCAAGTTCGTGCTGAAGGCGTCGCTGAGCGCCCCTGGC |
| | | AGCGAGTACGGCAGCCCGTCGGTCATCAGCGTCAGCAAAGGCA |
| | | GCCCTGACGGCAGCCACCCGGTGGTGGTGGCGCCCTACAACGG |
| | | CGGGCCGCCGCGCACGTGCCCCAAGATCAAGCAGGAGGCGGTC |
| | | TCTTCGTGCACCCACTTGGGCGCTGGACCCCCTCTCAGCAATG |
| | | GCCACCGGCCGGCTGCACACGACTTCCCCCTGGGGCGGCAGCT |
| | | CCCCAGCAGGACTACCCCGACCCTGGGTCTTGAGGAAGTGCTG |
| | | AGCAGCAGGGACTGTCACCCTGCCCTGCCGCTTCCTCCCGGCT |
| | | TCCATCCCCACCCGGGGCCCAATTACCCATCCTTCCTGCCCGA |
| | | TCAGATGCAGCCGCAAGTCCCGCCGCTCCATTACCAAGAGCTC |
| | | ATGCCACCCGGTTCCTGCATGCCAGAGGAGCCCAAGCCAAAGA |
| | | GGGGAAGACGATCGTGGCCCCGGAAAAGGACCGCCACCCACAC |
| | | TTGTGATTACGCGGGCTGCGGCAAAACCTACACAAAGAGTTCC |
| | | CATCTCAAGGCACACCTGCGAACCCACACAGGTGAGAAACCTT |
| | | ACCACTGTGACTGGGACGGCTGTGGATGGAAATTCGCCCGCTC |
| | | AGATGAACTGACCAGGCACTACCGTAAACACACGGGGCACCGC |
| | | CCGTTCCAGTGCCAAAAATGCGACCGAGCATTTTCCAGGTCGG |
| | | ACCACCTCGCCTTACACATGAAGAGGCATTTTTAAATGACTAG |
| | | TGCGCGCAGCGGCCGACCATGGCCCAACTTGTTTATTGCAGCT |
| | | TATAATGGTTACAAATAAAGCAATAGCATCACAAATTTCACAA |
| | | ATAAAGCATTTTTTTCACTGCATTCTAGTTGTGGTTTGTCCAA |
| | | ACTCATCAATGTATCTTATCATGTCTGGATCTCGGTACCGGAT |
| | | CCAAATTCCCGATAAGGATCTTCCTAGAGCATGGCTACGTAGA |
| | | TAAGTAGCATGGCGGGTTAATCATTAACTACAAGGAACCCCTA |
| | | GTGATGGAGTTGGCCACTCCCTCTCTGCGCGCTCGCTCGCTCA |
| | | CTGAGGCCGGGCGACCAAAGGTCGCCCGACGCCCGGGCTTTGC |
| | | CCGGGCGGCCTCAGTGAGCGAGCGAGCGCGCAGCCTTAATTAA |
| | | CCTAATTCATCCCAATGGCGCGCCGAGCTTGGCTCGAGCATGG |
| | | TCATAGCTGTTTCCTGTGTGAAATTGTTATCCGCTCACAATTC |
| | | CACACAACATACGAGCCGGAAGCATAAAGTGTAAAGCCTGGGG |
| | | TGCCTAATGAGTGAGCTAACTCACATTAATTGCGTTGCGCTCA |
| | | CTGCCCGCTTTCCAGTCGGGAAACCTGTCGTGCCAGCTGCATT |
| | | AATGAATCGGCCAACGCGCGGGGAGAGGCGGTTTGCGTATTGG |
| | | GCGCTGTTCCGCTTCCTCGCTCACTGACTCGCTGCGCTCGGTC |
| | | GTTCGGCTGCGGCGAGCGGTATCAGCTCACTCAAAGGCGGTAA |
| | | TACGGTTATCCACAGAATCAGGGGATAACGCAGGAAAGAACAT |
| | | GTGAGCAAAAGGCCAGCAAAAGGCCAGGAACCGTAAAAAGGCC |
| | | GCGTTGCTGGCGTTTTTCCATAGGCTCCGCCCCCCTGACGAGC |
| | | ATCACAAAAATCGACGCTCAAGTCAGAGGTGGCGAAACCCGAC |
| | | AGGACTATAAAGATACCAGGCGTTTCCCCCTGGAAGCTCCCTC |
| | | GTGCGCTCTCCTGTTCCGACCCTGCCGCTTACCGGATACCTGT |
| | | CCGCCTTTCTCCCTTCGGGAAGCGTGGCGCTTTCTCATAGCTC |
| | | ACGCTGTAGGTATCTCAGTTCGGTGTAGGTCGTTCGCTCCAAG |
| | | CTGGGCTGTGTGCACGAACCCCCCGTTCAGCCCGACCGCTGCG |
| | | CCTTATCCGGTAACTATCGTCTTGAGTCCAACCCGGTAAGACA |
| | | CGACTTATCGCCACTGGCAGCAGCCACTGGTAACAGGATTAGC |
| | | AGAGCGAGGTATGTAGGCGGTGCTACAGAGTTCTTGAAGTGGT |
| | | GGCCTAACTACGGCTACACTAGAAGAACAGTATTTGGTATCTG |
| | | CGCTCTGCTGAAGCCAGTTACCTTCGGAAAAAGAGTTGGTAGC |
| | | TCTTGATCCGGCAAACAAACCACCGCTGGTAGCGGTGGTTTTT |
| | | TTGTTTGCAAGCAGCAGATTACGCGCAGAAAAAAAGGATCTCA |
| | | AGAAGATCCTTTGATCTTTTCTACGGGGTCTGACGCTCAGTGG |
| | | AACGAAAACTCACGTTAAGGGATTTTGGTCATGAGATTATCAA |
| | | AAAGGATCTTCACCTAGATCCTTTTAAATTAAAAATGAAGTTT |
| | | TAAATCAATCTAAAGTATATATGAGTAAACTTGGTCTGACAGT |
| | | TAGAAAAACTCATCGAGCATCAAATGAAACTGCAATTTATTCA |
| | | TATCAGGATTATCAATACCATATTTTTGAAAAAGCCGTTTCTG |
| | | TAATGAAGGAGAAAACTCACCGAGGCAGTTCCATAGGATGGCA |
| | | AGATCCTGGTATCGGTCTGCGATTCCGACTCGTCCAACATCAA |
| | | TACAACCTATTAATTTCCCCTCGTCAAAAATAAGGTTATCAAG |
| | | TGAGAAATCACCATGAGTGACGACTGAATCCGGTGAGAATGGC |
| | | AAAAGTTTATGCATTTCTTTCCAGACTTGTTCAACAGGCCAGC |
| | | CATTACGCTCGTCATCAAAATCACTCGCATCAACCAAACCGTT |
| | | ATTCATTCGTGATTGCGCCTGAGCGAAACGAAATACGCGATCG |
| | | CTGTTAAAAGGACAATTACAAACAGGAATCGAATGCAACCGGC |
| | | GCAGGAACACTGCCAGCGCATCAACAATATTTTCACCTGAATC |
| | | AGGATATTCTTCTAATACCTGGAATGCTGTTTTCCCAGGGATC |
| | | GCAGTGGTGAGTAACCATGCATCATCAGGAGTACGGATAAAAT |
| | | GCTTGATGGTCGGAAGAGGCATAAATTCCGTCAGCCAGTTTAG |
| | | TCTGACCATCTCATCTGTAACATCATTGGCAACGCTACCTTTG |
| | | CCATGTTTCAGAAACAACTCTGGCGCATCGGGCTTCCCATACA |
| | | ATCGATAGATTGTCGCACCTGATTGCCCGACATTATCGCGAGC |
| | | CCATTTATACCCATATAAATCAGCATCCATGTTGGAATTTAAT |
| | | CGCGGCCTAGAGCAAGACGTTTCCCGTTGAATATGGCTCATAC |
| | | TCTTCCTTTTTCAATATTATTGAAGCATTTATCAGGGTTATTG |
| | | TCTCATGAGCGGATACATATTTGAATGTATTTAGAAAAATAAA |
| | | CAAATAGGGGTTCCGCGCACATTTCCCCGAAAAGTGCCACCTG |
| | | ACGTCTAAGAAACCATTATTATCATGACATTAACCTATAAAAA |
| | | TAGGCGTATCACGAGGCCCTTTTGTC |
| 16 | ITR - | CCTTAATTAGGCTGCGCGCTCGCTCGCTCACTGAGGCCGCCCG |
| | forward | GGCAAAGCCCGGGCGTCGGGCGACCTTTGGTCGCCCGGCCTCA |
| | | GTGAGCGAGCGAGCGCGCAGAGAGGGAGTGGCCAACTCCATCA |
| | | CTAGGGGTTCCT |
| 32 | ITR - reverse | AGGAACCCCTAGTGATGGAGTTGGCCACTCCCTCTCTGCGCGC |
| | | TCGCTCGCTCACTGAGGCCGGGCGACCAAAGGTCGCCCGACGC |
| | | CCGGGCTTTGCCCGGGCGGCCTCAGTGAGCGAGCGAGCGCGCA |
| | | GCTGCCTGCAGG |
| 35 | OCT4-2A- | CCTTAATTAGGCTGCGCGCTCGCTCGCTCACTGAGGCCGCCCG |
| | SOX2-2A- | GGCAAAGCCCGGGCGTCGGGCGACCTTTGGTCGCCCGGCCTCA |
| | KLF4 | GTGAGCGAGCGAGCGCGCAGAGAGGGAGTGGCCAACTCCATCA |
| | (Whole insert | CTAGGGGTTCCTTGTAGTTAATGATTAACCCGCCATGCTACTT |
| | sequence | ATCTACGTAGCCATGCTCTAGGAAGATCGGAATTCTTTACTCC |
| | including Tet | CTATCAGTGATAGAGAACGTATGAAGAGTTTACTCCCTATCAG |
| | operators, | TGATAGAGAACGTATGCAGACTTTACTCCCTATCAGTGATAGA |
| | SV40, and | GAACGTATAAGGAGTTTACTCCCTATCAGTGATAGAGAACGTA |
| | ITRs) | TGACCAGTTTACTCCCTATCAGTGATAGAGAACGTATCTACAG |
| | | TTTACTCCCTATCAGTGATAGAGAACGTATATCCAGTTTACTC |
| | | CCTATCAGTGATAGAGAACGTATAAGCTTTAGGCGTGTACGGT |
| | | GGGCGCCTATAAAAGCAGAGCTCGTTTAGTGAACCGTCAGATC |
| | | GCCTGGAGCAATTCCACAACACTTTTGTCTTATACCAACTTTC |
| | | CGTACCACTTCCTACCCTCGTAAAGCGGCCGCGCCACCATGGC |
| | | GGGACACCTGGCTTCGGATTTCGCCTTCTCGCCCCCTCCAGGT |
| | | GGTGGAGGTGATGGGCCAGGGGGGCCGGAGCCGGGCTGGGTTG |
| | | ATCCTCGGACCTGGCTAAGCTTCCAAGGCCCTCCTGGAGGGCC |
| | | AGGAATCGGGCCGGGGGTTGGGCCAGGCTCTGAGGTGTGGGGG |
| | | ATTCCCCCATGCCCCCCGCCGTATGAGTTCTGTGGGGGGATGG |
| | | CGTACTGTGGGCCCCAGGTTGGAGTGGGGCTAGTGCCCCAAGG |
| | | CGGCTTGGAGACCTCTCAGCCTGAGGGCGAAGCAGGAGTCGGG |
| | | GTGGAGAGCAACTCCGATGGGGCCTCCCCGGAGCCCTGCACCG |
| | | TCACCCCTGGTGCCGTGAAGCTGGAGAAGGAGAAGCTGGAGCA |
| | | AAACCCGGAGGAGTCCCAGGACATCAAAGCTCTGCAGAAAGAA |
| | | CTCGAGCAATTTGCCAAGCTCCTGAAGCAGAAGAGGATCACCC |
| | | TGGGATATACACAGGCCGATGTGGGGCTCACCCTGGGGGTTCT |
| | | ATTTGGGAAGGTATTCAGCCAAACGACCATCTGCCGCTTTGAG |
| | | GCTCTGCAGCTTAGCTTCAAGAACATGTGTAAGCTGCGGCCCT |
| | | TGCTGCAGAAGTGGGTGGAGGAAGCTGACAACAATGAAAATCT |
| | | TCAGGAGATATGCAAAGCAGAAACCCTCGTGCAGGCCCGAAAG |
| | | AGAAAGCGAACCAGTATCGAGAACCGAGTGAGAGGCAACCTGG |
| | | AGAATTTGTTCCTGCAGTGCCCGAAACCCACACTGCAGCAGAT |
| | | CAGCCACATCGCCCAGCAGCTTGGGCTCGAGAAGGATGTGGTC |
| | | CGAGTGTGGTTCTGTAACCGGCGCCAGAAGGGCAAGCGATCAA |
| | | GCAGCGACTATGCACAACGAGAGGATTTTGAGGCTGCTGGGTC |
| | | TCCTTTCTCAGGGGGACCAGTGTCCTTTCCTCTGGCCCCAGGG |
| | | CCCCATTTTGGTACCCCAGGCTATGGGAGCCCTCACTTCACTG |
| | | CACTGTACTCCTCGGTCCCTTTCCCTGAGGGGGAAGCCTTTCC |
| | | CCCTGTCTCTGTCACCACTCTGGGCTCTCCCATGCATTCAAAC |
| | | GCTAGCGGCAGCGGCGCCACGAACTTCTCTCTGTTAAAGCAAG |
| | | CAGGAGATGTTGAAGAAAACCCCGGGCCTGCATGCATGTACAA |
| | | CATGATGGAGACGGAGCTGAAGCCGCCGGGCCCGCAGCAAACT |
| | | TCGGGGGGCGGCGGCGGCAACTCCACCGCGGCGGCGGCCGGCG |
| | | GCAACCAGAAAAACAGCCCGGACCGCGTCAAGCGGCCCATGAA |
| | | TGCCTTCATGGTGTGGTCCCGCGGGCAGCGGCGCAAGATGGCC |
| | | CAGGAGAACCCCAAGATGCACAACTCGGAGATCAGCAAGCGCC |
| | | TGGGCGCCGAGTGGAAACTTTTGTCGGAGACGGAGAAGCGGCC |
| | | GTTCATCGACGAGGCTAAGCGGCTGCGAGCGCTGCACATGAAG |
| | | GAGCACCCGGATTATAAATACCGGCCCCGGCGGAAAACCAAGA |
| | | CGCTCATGAAGAAGGATAAGTACACGCTGCCCGGCGGGCTGCT |
| | | GGCCCCCGGCGGCAATAGCATGGCGAGCGGGGTCGGGGTGGGC |
| | | GCCGGCCTGGGCGCGGGCGTGAACCAGCGCATGGACAGTTACG |
| | | CGCACATGAACGGCTGGAGCAACGGCAGCTACAGCATGATGCA |
| | | GGACCAGCTGGGCTACCCGCAGCACCCGGGCCTCAATGCGCAC |
| | | GGCGCAGCGCAGATGCAGCCCATGCACCGCTACGACGTGAGCG |
| | | CCCTGCAGTACAACTCCATGACCAGCTCGCAGACCTACATGAA |
| | | CGGCTCGCCCACCTACAGCATGTCCTACTCGCAGCAGGGCACC |
| | | CCTGGCATGGCTCTTGGCTCCATGGGTTCGGTGGTCAAGTCCG |
| | | AGGCCAGCTCCAGCCCCCCTGTGGTTACCTCTTCCTCCCACTC |
| | | CAGGGCGCCCTGCCAGGCCGGGGACCTCCGGGACATGATCAGC |
| | | ATGTATCTCCCCGGCGCCGAGGTGCCGGAACCCGCCGCCCCCA |
| | | GCAGACTTCACATGTCCCAGCACTACCAGAGCGGCCCGGTGCC |
| | | CGGCACGGCCATTAACGGCACACTGCCCCTCTCACACATGGCA |
| | | TGCGGCTCCGGCGAGGGCAGGGGAAGTCTTCTAACATGCGGGG |
| | | ACGTGGAGGAAAATCCCGGCCCACTCGAGATGGCTGTCAGCGA |
| | | CGCGCTGCTCCCATCTTTCTCCACGTTCGCGTCTGGCCCGGCG |
| | | GGAAGGGAGAAGACACTGCGTCAAGCAGGTGCCCCGAATAACC |
| | | GCTGGCGGGAGGAGCTCTCCCACATGAAGCGACTTCCCCCAGT |
| | | GCTTCCCGGCCGCCCCTATGACCTGGCGGCGGCGACCGTGGCC |
| | | ACAGACCTGGAGAGCGGCGGAGCCGGTGCGGCTTGCGGCGGTA |
| | | GCAACCTGGCGCCCCTACCTCGGAGAGAGACCGAGGAGTTCAA |
| | | CGATCTCCTGGACCTGGACTTTATTCTCTCCAATTCGCTGACC |
| | | CATCCTCCGGAGTCAGTGGCCGCCACCGTGTCCTCGTCAGCGT |
| | | CAGCCTCCTCTTCGTCGTCGCCGTCGAGCAGCGGCCCTGCCAG |
| | | CGCGCCCTCCACCTGCAGCTTCACCTATCCGATCCGGGCCGGG |
| | | AACGACCCGGGCGTGGCGCCGGGCGGCACGGGCGGAGGCCTCC |
| | | TCTATGGCAGGGAGTCCGCTCCCCCTCCGACGGCTCCCTTCAA |
| | | CCTGGCGGACATCAACGACGTGAGCCCCTCGGGCGGCTTCGTG |
| | | GCCGAGCTCCTGCGGCCAGAATTGGACCCGGTGTACATTCCGC |
| | | CGCAGCAGCCGCAGCCGCCAGGTGGCGGGCTGATGGGCAAGTT |
| | | CGTGCTGAAGGCGTCGCTGAGCGCCCCTGGCAGCGAGTACGGC |
| | | AGCCCGTCGGTCATCAGCGTCAGCAAAGGCAGCCCTGACGGCA |
| | | GCCACCCGGTGGTGGTGGCGCCCTACAACGGCGGGCCGCCGCG |
| | | CACGTGCCCCAAGATCAAGCAGGAGGCGGTCTCTTCGTGCACC |
| | | CACTTGGGCGCTGGACCCCCTCTCAGCAATGGCCACCGGCCGG |
| | | CTGCACACGACTTCCCCCTGGGGCGGCAGCTCCCCAGCAGGAC |
| | | TACCCCGACCCTGGGTCTTGAGGAAGTGCTGAGCAGCAGGGAC |
| | | TGTCACCCTGCCCTGCCGCTTCCTCCCGGCTTCCATCCCCACC |
| | | CGGGGCCCAATTACCCATCCTTCCTGCCCGATCAGATGCAGCC |
| | | GCAAGTCCCGCCGCTCCATTACCAAGAGCTCATGCCACCCGGT |
| | | TCCTGCATGCCAGAGGAGCCCAAGCCAAAGAGGGGAAGACGAT |
| | | CGTGGCCCCGGAAAAGGACCGCCACCCACACTTGTGATTACGC |
| | | GGGCTGCGGCAAAACCTACACAAAGAGTTCCCATCTCAAGGCA |
| | | CACCTGCGAACCCACACAGGTGAGAAACCTTACCACTGTGACT |
| | | GGGACGGCTGTGGATGGAAATTCGCCCGCTCAGATGAACTGAC |
| | | CAGGCACTACCGTAAACACACGGGGCACCGCCCGTTCCAGTGC |
| | | CAAAAATGCGACCGAGCATTTTCCAGGTCGGACCACCTCGCCT |
| | | TACACATGAAGAGGCATTTTTAAATGACTAGTGCGCGCAGCGG |
| | | CCGACCATGGCCCAACTTGTTTATTGCAGCTTATAATGGTTAC |
| | | AAATAAAGCAATAGCATCACAAATTTCACAAATAAAGCATTTT |
| | | TTTCACTGCATTCTAGTTGTGGTTTGTCCAAACTCATCAATGT |
| | | ATCTTATCATGTCTGGATCTCGGTACCGGATCCAAATTCCCGA |
| | | TAAGGATCTTCCTAGAGCATGGCTACGTAGATAAGTAGCATGG |
| | | CGGGTTAATCATTAACTACAAGGAACCCCTA |

TABLE 7
AAV2-CMV-rtTA3V16 Vector Sequences

| SEQ | | |
|---|---|---|
| ID NO: | Component | Sequence |
| 17 | CMV promoter | GTGATGCGGTTTTGGCAGTACATCAATGGGCGTGGATAGCG |
| | | GTTTGACTCACGGGGATTTCCAAGTCTCCACCCCATTGACG |
| | | TCAATGGGAGTTTGTTTTGGCACCAAAATCAACGGGACTTT |
| | | CCAAAATGTCGTAACAACTCCGCCCCATTGACGCAAATGG |
| | | GCGGTAGGCGTGTACGGTGGGAGGTCTATATAAGCAGAGC |
| | | T |
| 18 | CMV enhancer | GACATTGATTATTGACTAGTTATTAATAGTAATCAATTACG |
| | | GGGTCATTAGTTCATAGCCCATATATGGAGTTCCGCGTTAC |
| | | ATAACTTACGGTAAATGGCCCGCCTGGCTGACCGCCCAACG |
| | | ACCCCCGCCCATTGACGTCAATAATGACGTATGTTCCCATA |
| | | GTAACGCCAATAGGGACTTTCCATTGACGTCAATGGGTGGA |
| | | GTATTTACGCTAAACTGCCCACTTGGCAGTACATCAAGTGT |
| | | ATCATATGCCAAGTACGCCCCCTATTGACGTCAATGACGGT |
| | | AAATGGCCCGCCTGGCATTATGCCCAGTACATGACCTTATG |
| | | GGACTTTCCTACTTGGCAGTACATCTACGTATTAGTCATCG |
| | | CTATTACCATG |
| 19 | rtTA3 Advanced | ATGTCTAGACTGGACAAGAGCAAAATCATAAACAGCGCTC |
| | nucleic acid | TGGAATTACTCAATGGAGTCGGTATCGAAGGCCTGACGAC |
| | sequence | AAGGAAACTCGCTCAAAAGCTGGGAGTTGAGCAGCCTACC |
| | | CTGTACTGGCACGTGAAGAACAAGCGGGCCCTGCTCGATG |
| | | CCCTGCCAATCGAGATGCTGGACAGGCATCATACCCACAG |
| | | CTGCCCCCTGGAAGGCGAGTCATGGCAAGACTTTCTGCGGA |
| | | ACAACGCCAAGTCATACCGCTGTGCTCTCCTCTCACATCGC |
| | | GACGGGGCTAAAGTGCATCTCGGCACCCGCCCAACAGAGA |
| | | AACAGTACGAAACCCTGGAAAATCAGCTCGCGTTCCTGTGT |
| | | CAGCAAGGCTTCTCCCTGGAGAACGCACTGTACGCTCTGTC |
| | | CGCCGTGGGCCACTTTACACTGGGCTGCGTATTGGAGGAAC |
| | | AGGAGCATCAAGTAGCAAAAGAGGAAAGAGAGACACCTA |
| | | CCACCGATTCTATGCCCCCACTTCTGAAGCAAGCAATTGAG |
| | | CTGTTCGACCGGCAGGGAGCCGAACCTGCCTTCCTTTTCGG |
| | | CCTGGAACTAATCATATGTGGCCTGGAGAAACAGCTAAAG |
| | | TGCGAAAGCGGCGGGCCGACCGACGCCCTTGACGATTTTG |
| | | ACTTAGACATGCTCCCAGCCGATGCCCTTGACGACTTTGAC |
| | | CTTGATATGCTGCCTGCTGACGCTCTTGACGATTTTGACCTT |
| | | GACATGCTCCCCGGGTAA |
| 20 | rtTA3 Advanced | MSRLDKSKIINSALELLNGVGIEGLTTRKLAQKLGVEQPTLYW |
| | amino acid | HVKNKRALLDALPIEMLDRHHTHSCPLEGESWQDFLRNNAKS |
| | | YRCALLSHRDGAKVHLGTRPTEKQYETLENQLAFLCQQGFSL |
| | | ENALYALSAVGHFTLGCVLEEQEHQVAKEERETPTTDSMPPL |
| | | LKQAIELFDRQGAEPAFLFGLELIICGLEKQLKCESGGPTDALD |
| | | DFDLDMLPADALDDFDLDMLPADALDDFDLDMLPG* |
| 21 | AAV2-CMV- | TCGCGCGTTTCGGTGATGACGGTGAAAACCTCTGACACATG |
| | rtTA3V16 (full | CAGCTCCCGGAGACTGTCACAGCTTGTCTGTAAGCGGATGC |
| | plasmid | CGGGAGCAGACAAGCCCGTCAGGGCGCGTCAGCGGGTGTT |
| | sequence) | GGCGGGTGTCGGGGCTGGCTTAACTATGCGGCATCAGAGC |
| | | AGATTGTACTGAGAGTGCACCATATGCGGTGTGAAATACC |
| | | GCACAGATGCGTAAGGAGAAAATACCGCATCAGGCGCCAT |
| | | TCGCCATTCAGGCTGCGCAACTGTTGGGAAGGGCGATCGGT |
| | | GCGGGCCTCTTCGCTATTACGCCAGCTGGCGAAAGGGGGA |
| | | TGTGCTGCAAGGCGATTAAGTTGGGTAACGCCAGGGTTTTC |
| | | CCAGTCACGACGTTGTAAAACGACGGCCAGTGAATTGACG |
| | | CGTATTGGGATACTATGGTTGCTTTGACGTATGCGGTGTGA |
| | | AATACCGCACAGATGCGTAAGGAGAAAATACCGCATCAGG |
| | | CGCCCCTGCAGGCAGCTGCGCGCTCGCTCGCTCACTGAGGC |
| | | CGCCCGGGCAAAGCCCGGGCGTCGGGCGACCTTTGGTCGC |
| | | CCGGCCTCAGTGAGCGAGCGAGCGCGCAGAGAGGGAGTGG |
| | | CCAACTCCATCACTAGGGGTTCCTGCGGCCGCTCGGTCCGC |
| | | ACGATCTCAATTCGGCCATTACGGCCGGATCCGGCTCGAGG |
| | | AGCTTGGCCCATTGCATACGTTGTATCCATATCATAATATG |
| | | TACATTTATATTGGCTCATGTCCAACATTACCGCCATGTTG |
| | | ACATTGATTATTGACTAGTTATTAATAGTAATCAATTACGG |
| | | GGTCATTAGTTCATAGCCCATATATGGAGTTCCGCGTTACA |
| | | TAACTTACGGTAAATGGCCCGCCTGGCTGACCGCCCAACGA |
| | | CCCCCGCCCATTGACGTCAATAATGACGTATGTTCCCATAG |
| | | TAACGCCAATAGGGACTTTCCATTGACGTCAATGGGTGGAG |
| | | TATTTACGCTAAACTGCCCACTTGGCAGTACATCAAGTGTA |
| | | TCATATGCCAAGTACGCCCCCTATTGACGTCAATGACGGTA |
| | | AATGGCCCGCCTGGCATTATGCCCAGTACATGACCTTATGG |
| | | GACTTTCCTACTTGGCAGTACATCTACGTATTAGTCATCGCT |
| | | ATTACCATGGTGATGCGGTTTTGGCAGTACATCAATGGGCG |
| | | TGGATAGCGGTTTGACTCACGGGGATTTCCAAGTCTCCACC |
| | | CCATTGACGTCAATGGGAGTTTGTTTTGGCACCAAAATCAA |
| | | CGGGACTTTCCAAAATGTCGTAACAACTCCGCCCCATTGAC |
| | | GCAAATGGGCGGTAGGCGTGTACGGTGGGAGGTCTATATA |
| | | AGCAGAGCTCGTTTAGTGAACCGTCAGATCGCCTGGAGAC |
| | | GCCATCCACGCTGTTTTGACCTCCATAGAAGACACCGGGAC |
| | | CGATCCAGCCTCCGCGGCCCCGAATTCACCATGTCTAGACT |
| | | GGACAAGAGCAAAATCATAAACAGCGCTCTGGAATTACTC |
| | | AATGGAGTCGGTATCGAAGGCCTGACGACAAGGAAACTCG |
| | | CTCAAAAGCTGGGAGTTGAGCAGCCTACCCTGTACTGGCAC |
| | | GTGAAGAACAAGCGGGCCCTGCTCGATGCCCTGCCAATCG |
| | | AGATGCTGGACAGGCATCATACCCACAGCTGCCCCCTGGA |
| | | AGGCGAGTCATGGCAAGACTTTCTGCGGAACAACGCCAAG |
| | | TCATACCGCTGTGCTCTCCTCTCACATCGCGACGGGGCTAA |
| | | AGTGCATCTCGGCACCCGCCCAACAGAGAAACAGTACGAA |
| | | ACCCTGGAAAATCAGCTCGCGTTCCTGTGTCAGCAAGGCTT |
| | | CTCCCTGGAGAACGCACTGTACGCTCTGTCCGCCGTGGGCC |
| | | ACTTTACACTGGGCTGCGTATTGGAGGAACAGGAGCATCA |
| | | AGTAGCAAAAGAGGAAAGAGAGACACCTACCACCGATTCT |
| | | ATGCCCCCACTTCTGAAGCAAGCAATTGAGCTGTTCGACCG |
| | | GCAGGGAGCCGAACCTGCCTTCCTTTTCGGCCTGGAACTAA |
| | | TCATATGTGGCCTGGAGAAACAGCTAAAGTGCGAAAGCGG |
| | | CGGGCCGACCGACGCCCTTGACGATTTTGACTTAGACATGC |
| | | TCCCAGCCGATGCCCTTGACGACTTTGACCTTGATATGCTG |
| | | CCTGCTGACGCTCTTGACGATTTTGACCTTGACATGCTCCCC |
| | | GGGTAACTAAGTAAGGATCATCTTAATTAAATCGATAAGG |
| | | ATCTGGCCGCCTCGGCCTAATCAACCTCTGGATTACAAAAT |
| | | TTGTGAAAGATTGACTGGTATTCTTAACTATGTTGCTCCTTT |
| | | TACGCTATGTGGATACGCTGCTTTAATGCCTTTGTATCATGC |
| | | TATTGCTTCCCGTATGGCTTTCATTTTCTCCTCCTTGTATAA |
| | | ATCCTGGTTGCTGTCTCTTTATGAGGAGTTGTGGCCCGTTGT |
| | | CAGGCAACGTGGCGTGGTGTGCACTGTGTTTGCTGACGCAA |
| | | CCCCCACTGGTTGGGGCATTGCCACCACCTGTCAGCTCCTT |
| | | TCCGGGACTTTCGCTTTCCCCCTCCCTATTGCCACGGCGGA |
| | | ACTCATCGCCGCCTGCCTTGCCCGCTGCTGGACAGGGGCTC |
| | | GGCTGTTGGGCACTGACAATTCCGTGGTGTTGTCGGGGAAA |
| | | TCATCGTCCTTTCCTTGGCTGCTCGCCTGTGTTGCCACCTGG |
| | | ATTCTGCGCGGGACGTCCTTCTGCTACGTCCCTTCGGCCCTC |
| | | AATCCAGCGGACCTTCCTTCCCGCGGCCTGCTGCCGGCTCT |
| | | GCGGCCTCTTCCGCGTCTTCGCCTTCGCCCTCAGACGAGTC |
| | | GGATCTCCCTTTGGGCCGCCTCCCCGCCAGACATGATAAGA |
| | | TACATTGATGAGTTTGGACAAACCACAACTAGAATGCAGT |
| | | GAAAAAAATGCTTTATTTGTGAAATTTGTGATGCTATTGCT |
| | | TTATTTGTAACCATTATAAGCTGCAATAAACAAGTTAACAA |
| | | CAACAATTGCATTCATTTTATGTTTCAGGTTCAGGGGGAGA |
| | | TGTGGGAGGTTTTTTAAAGCAAGTAAAACCTCTACAAATGT |
| | | GGTAACTAGCGCGTGCGGCCGCAGGAACCCCTAGTGATGG |
| | | AGTTGGCCACTCCCTCTCTGCGCGCTCGCTCGCTCACTGAG |
| | | GCCGGGCGACCAAAGGTCGCCCGACGCCCGGGCTTTGCCC |
| | | GGGCGGCCTCAGTGAGCGAGCGAGCGCGCAGCTGCCTGCA |
| | | GGATCCCAATGGCGCGCCGAGCTTGGCTCGAGCATGGTCAT |
| | | AGCTGTTTCCTGTGTGAAATTGTTATCCGCTCACAATTCCAC |
| | | ACAACATACGAGCCGGAAGCATAAAGTGTAAAGCCTGGGG |
| | | TGCCTAATGAGTGAGCTAACTCACATTAATTGCGTTGCGCT |
| | | CACTGCCCGCTTTCCAGTCGGGAAACCTGTCGTGCCAGCTG |
| | | CATTAATGAATCGGCCAACGCGCGGGGAGAGGCGGTTTGC |
| | | GTATTGGGCGCTGTTCCGCTTCCTCGCTCACTGACTCGCTGC |
| | | GCTCGGTCGTTCGGCTGCGGCGAGCGGTATCAGCTCACTCA |
| | | AAGGCGGTAATACGGTTATCCACAGAATCAGGGGATAACG |
| | | CAGGAAAGAACATGTGAGCAAAAGGCCAGCAAAAGGCCA |
| | | GGAACCGTAAAAAGGCCGCGTTGCTGGCGTTTTTCCATAGG |
| | | CTCCGCCCCCCTGACGAGCATCACAAAAATCGACGCTCAA |
| | | GTCAGAGGTGGCGAAACCCGACAGGACTATAAAGATACCA |
| | | GGCGTTTCCCCCTGGAAGCTCCCTCGTGCGCTCTCCTGTTCC |
| | | GACCCTGCCGCTTACCGGATACCTGTCCGCCTTTCTCCCTTC |
| | | GGGAAGCGTGGCGCTTTCTCATAGCTCACGCTGTAGGTATC |
| | | TCAGTTCGGTGTAGGTCGTTCGCTCCAAGCTGGGCTGTGTG |
| | | CACGAACCCCCCGTTCAGCCCGACCGCTGCGCCTTATCCGG |
| | | TAACTATCGTCTTGAGTCCAACCCGGTAAGACACGACTTAT |
| | | CGCCACTGGCAGCAGCCACTGGTAACAGGATTAGCAGAGC |
| | | GAGGTATGTAGGCGGTGCTACAGAGTTCTTGAAGTGGTGG |
| | | CCTAACTACGGCTACACTAGAAGAACAGTATTTGGTATCTG |
| | | CGCTCTGCTGAAGCCAGTTACCTTCGGAAAAAGAGTTGGTA |
| | | GCTCTTGATCCGGCAAACAAACCACCGCTGGTAGCGGTGGT |
| | | TTTTTTGTTTGCAAGCAGCAGATTACGCGCAGAAAAAAAGG |
| | | ATCTCAAGAAGATCCTTTGATCTTTTCTACGGGGTCTGACG |
| | | CTCAGTGGAACGAAAACTCACGTTAAGGGATTTTGGTCATG |
| | | AGATTATCAAAAAGGATCTTCACCTAGATCCTTTTAAATTA |
| | | AAAATGAAGTTTTAAATCAATCTAAAGTATATATGAGTAAA |
| | | CTTGGTCTGACAGTTAGAAAAACTCATCGAGCATCAAATGA |
| | | AACTGCAATTTATTCATATCAGGATTATCAATACCATATTTT |
| | | TGAAAAAGCCGTTTCTGTAATGAAGGAGAAAACTCACCGA |
| | | GGCAGTTCCATAGGATGGCAAGATCCTGGTATCGGTCTGCG |
| | | ATTCCGACTCGTCCAACATCAATACAACCTATTAATTTCCC |
| | | CTCGTCAAAAATAAGGTTATCAAGTGAGAAATCACCATGA |
| | | GTGACGACTGAATCCGGTGAGAATGGCAAAAGTTTATGCA |
| | | TTTCTTTCCAGACTTGTTCAACAGGCCAGCCATTACGCTCGT |
| | | CATCAAAATCACTCGCATCAACCAAACCGTTATTCATTCGT |
| | | GATTGCGCCTGAGCGAAACGAAATACGCGATCGCTGTTAA |
| | | AAGGACAATTACAAACAGGAATCGAATGCAACCGGCGCAG |
| | | GAACACTGCCAGCGCATCAACAATATTTTCACCTGAATCAG |
| | | GATATTCTTCTAATACCTGGAATGCTGTTTTCCCAGGGATC |
| | | GCAGTGGTGAGTAACCATGCATCATCAGGAGTACGGATAA |
| | | AATGCTTGATGGTCGGAAGAGGCATAAATTCCGTCAGCCA |
| | | GTTTAGTCTGACCATCTCATCTGTAACATCATTGGCAACGC |
| | | TACCTTTGCCATGTTTCAGAAACAACTCTGGCGCATCGGGC |
| | | TTCCCATACAATCGATAGATTGTCGCACCTGATTGCCCGAC |
| | | ATTATCGCGAGCCCATTTATACCCATATAAATCAGCATCCA |
| | | TGTTGGAATTTAATCGCGGCCTAGAGCAAGACGTTTCCCGT |
| | | TGAATATGGCTCATACTCTTCCTTTTTCAATATTATTGAAGC |
| | | ATTTATCAGGGTTATTGTCTCATGAGCGGATACATATTTGA |
| | | ATGTATTTAGAAAAATAAACAAATAGGGGTTCCGCGCACA |
| | | TTTCCCCGAAAAGTGCCACCTGACGTCTAAGAAACCATTAT |
| | | TATCATGACATTAACCTATAAAAATAGGCGTATCACGAGGC |
| | | CCTTTTGTC |
| 22 | ITR-forward | CCTGCAGGCAGCTGCGCGCTCGCTCGCTCACTGAGGCCGCC |
| | | CGGGCAAAGCCCGGGCGTCGGGCGACCTTTGGTCGCCCGG |
| | | CCTCAGTGAGCGAGCGAGCGCGCAGAGAGGGAGTGGCCAA |
| | | CTCCATCACTAGGGGTTCCT |
| 33 | ITR-reverse | AGGAACCCCTAGTGATGGAGTTGGCCACTCCCTCTCTGCGC |
| | | GCTCGCTCGCTCACTGAGGCCGGGCGACCAAAGGTCGCCC |
| | | GACGCCCGGGCTTTGCCCGGGCGGCCTCAGTGAGCGAGCG |
| | | AGCGCGCAGCTGCCTGCAGG |
| 23 | WPRE | AATCAACCTCTGGATTACAAAATTTGTGAAAGATTGACTGG |
| | | TATTCTTAACTATGTTGCTCCTTTTACGCTATGTGGATACGC |
| | | TGCTTTAATGCCTTTGTATCATGCTATTGCTTCCCGTATGGC |
| | | TTTCATTTTCTCCTCCTTGTATAAATCCTGGTTGCTGTCTCTT |
| | | TATGAGGAGTTGTGGCCCGTTGTCAGGCAACGTGGCGTGGT |
| | | GTGCACTGTGTTTGCTGACGCAACCCCCACTGGTTGGGGCA |
| | | TTGCCACCACCTGTCAGCTCCTTTCCGGGACTTTCGCTTTCC |
| | | CCCTCCCTATTGCCACGGCGGAACTCATCGCCGCCTGCCTT |
| | | GCCCGCTGCTGGACAGGGGCTCGGCTGTTGGGCACTGACA |
| | | ATTCCGTGGTGTTGTCGGGGAAATCATCGTCCTTTCCTTGG |
| | | CTGCTCGCCTGTGTTGCCACCTGGATTCTGCGCGGGACGTC |
| | | CTTCTGCTACGTCCCTTCGGCCCTCAATCCAGCGGACCTTCC |
| | | TTCCCGCGGCCTGCTGCCGGCTCTGCGGCCTCTTCCGCGTCT |
| | | TCGCCTTCGCCCTCAGACGAGTCGGATCTCCCTTTGGGCCG |
| | | CCTCCCCGC |
| 36 | Full CMV- | CCTGCAGGCAGCTGCGCGCTCGCTCGCTCACTGAGGCCGCC |
| | rtTA3V16 | CGGGCAAAGCCCGGGCGTCGGGCGACCTTTGGTCGCCCGG |
| | sequence | CCTCAGTGAGCGAGCGAGCGCGCAGAGAGGGAGTGGCCAA |
| | (including ITRs) | CTCCATCACTAGGGGTTCCTGCGGCCGCTCGGTCCGCACGA |
| | | TCTCAATTCGGCCATTACGGCCGGATCCGGCTCGAGGAGCT |
| | | TGGCCCATTGCATACGTTGTATCCATATCATAATATGTACA |
| | | TTTATATTGGCTCATGTCCAACATTACCGCCATGTTGACATT |
| | | GATTATTGACTAGTTATTAATAGTAATCAATTACGGGGTCA |
| | | TTAGTTCATAGCCCATATATGGAGTTCCGCGTTACATAACT |
| | | TACGGTAAATGGCCCGCCTGGCTGACCGCCCAACGACCCCC |
| | | GCCCATTGACGTCAATAATGACGTATGTTCCCATAGTAACG |
| | | CCAATAGGGACTTTCCATTGACGTCAATGGGTGGAGTATTT |
| | | ACGCTAAACTGCCCACTTGGCAGTACATCAAGTGTATCATA |
| | | TGCCAAGTACGCCCCCTATTGACGTCAATGACGGTAAATGG |
| | | CCCGCCTGGCATTATGCCCAGTACATGACCTTATGGGACTT |
| | | TCCTACTTGGCAGTACATCTACGTATTAGTCATCGCTATTAC |
| | | CATGGTGATGCGGTTTTGGCAGTACATCAATGGGCGTGGAT |
| | | AGCGGTTTGACTCACGGGGATTTCCAAGTCTCCACCCCATT |
| | | GACGTCAATGGGAGTTTGTTTTGGCACCAAAATCAACGGG |
| | | ACTTTCCAAAATGTCGTAACAACTCCGCCCCATTGACGCAA |
| | | ATGGGCGGTAGGCGTGTACGGTGGGAGGTCTATATAAGCA |
| | | GAGCTCGTTTAGTGAACCGTCAGATCGCCTGGAGACGCCAT |
| | | CCACGCTGTTTTGACCTCCATAGAAGACACCGGGACCGATC |
| | | CAGCCTCCGCGGCCCCGAATTCACCATGTCTAGACTGGACA |
| | | AGAGCAAAATCATAAACAGCGCTCTGGAATTACTCAATGG |
| | | AGTCGGTATCGAAGGCCTGACGACAAGGAAACTCGCTCAA |
| | | AAGCTGGGAGTTGAGCAGCCTACCCTGTACTGGCACGTGA |
| | | AGAACAAGCGGGCCCTGCTCGATGCCCTGCCAATCGAGAT |
| | | GCTGGACAGGCATCATACCCACAGCTGCCCCCTGGAAGGC |
| | | GAGTCATGGCAAGACTTTCTGCGGAACAACGCCAAGTCAT |
| | | ACCGCTGTGCTCTCCTCTCACATCGCGACGGGGCTAAAGTG |
| | | CATCTCGGCACCCGCCCAACAGAGAAACAGTACGAAACCC |
| | | TGGAAAATCAGCTCGCGTTCCTGTGTCAGCAAGGCTTCTCC |
| | | CTGGAGAACGCACTGTACGCTCTGTCCGCCGTGGGCCACTT |
| | | TACACTGGGCTGCGTATTGGAGGAACAGGAGCATCAAGTA |
| | | GCAAAAGAGGAAAGAGAGACACCTACCACCGATTCTATGC |
| | | CCCCACTTCTGAAGCAAGCAATTGAGCTGTTCGACCGGCAG |
| | | GGAGCCGAACCTGCCTTCCTTTTCGGCCTGGAACTAATCAT |
| | | ATGTGGCCTGGAGAAACAGCTAAAGTGCGAAAGCGGCGGG |
| | | CCGACCGACGCCCTTGACGATTTTGACTTAGACATGCTCCC |
| | | AGCCGATGCCCTTGACGACTTTGACCTTGATATGCTGCCTG |
| | | CTGACGCTCTTGACGATTTTGACCTTGACATGCTCCCCGGG |
| | | TAACTAAGTAAGGATCATCTTAATTAAATCGATAAGGATCT |
| | | GGCCGCCTCGGCCTAATCAACCTCTGGATTACAAAATTTGT |
| | | GAAAGATTGACTGGTATTCTTAACTATGTTGCTCCTTTTACG |
| | | CTATGTGGATACGCTGCTTTAATGCCTTTGTATCATGCTATT |
| | | GCTTCCCGTATGGCTTTCATTTTCTCCTCCTTGTATAAATCC |
| | | TGGTTGCTGTCTCTTTATGAGGAGTTGTGGCCCGTTGTCAG |
| | | GCAACGTGGCGTGGTGTGCACTGTGTTTGCTGACGCAACCC |
| | | CCACTGGTTGGGGCATTGCCACCACCTGTCAGCTCCTTTCC |
| | | GGGACTTTCGCTTTCCCCCTCCCTATTGCCACGGCGGAACT |
| | | CATCGCCGCCTGCCTTGCCCGCTGCTGGACAGGGGCTCGGC |
| | | TGTTGGGCACTGACAATTCCGTGGTGTTGTCGGGGAAATCA |
| | | TCGTCCTTTCCTTGGCTGCTCGCCTGTGTTGCCACCTGGATT |
| | | CTGCGCGGGACGTCCTTCTGCTACGTCCCTTCGGCCCTCAA |
| | | TCCAGCGGACCTTCCTTCCCGCGGCCTGCTGCCGGCTCTGC |
| | | GGCCTCTTCCGCGTCTTCGCCTTCGCCCTCAGACGAGTCGG |
| | | ATCTCCCTTTGGGCCGCCTCCCCGCCAGACATGATAAGATA |
| | | CATTGATGAGTTTGGACAAACCACAACTAGAATGCAGTGA |
| | | AAAAAATGCTTTATTTGTGAAATTTGTGATGCTATTGCTTTA |
| | | TTTGTAACCATTATAAGCTGCAATAAACAAGTTAACAACAA |
| | | CAATTGCATTCATTTTATGTTTCAGGTTCAGGGGGAGATGT |
| | | GGGAGGTTTTTTAAAGCAAGTAAAACCTCTACAAATGTGGT |
| | | AACTAGCGCGTGCGGCCGCAGGAACCCCTAGTGATGGAGT |
| | | TGGCCACTCCCTCTCTGCGCGCTCGCTCGCTCACTGAGGCC |
| | | GGGCGACCAAAGGTCGCCCGACGCCCGGGCTTTGCCCGGG |
| | | CGGCCTCAGTGAGCGAGCGAGCGCGCAGCTGCCTGCAGG |

TABLE 8
AAV2-CMV-rtTA4 V16 Vector Sequences

| SEQ | | |
|---|---|---|
| ID NO: | Component | Sequence |
| 24 | CMV | GTGATGCGGTTTTGGCAGTACATCAATGGGCGTGGATAGCGGT |
| | promoter | TTGACTCACGGGGATTTCCAAGTCTCCACCCCATTGACGTCAAT |
| | | GGGAGTTTGTTTTGGCACCAAAATCAACGGGACTTTCCAAAAT |
| | | GTCGTAACAACTCCGCCCCATTGACGCAAATGGGCGGTAGGCG |
| | | TGTACGGTGGGAGGTCTATATAAGCAGAGCT |
| 25 | CMV | GACATTGATTATTGACTAGTTATTAATAGTAATCAATTACGGG |
| | enhancer | GTCATTAGTTCATAGCCCATATATGGAGTTCCGCGTTACATAAC |
| | | TTACGGTAAATGGCCCGCCTGGCTGACCGCCCAACGACCCCCG |
| | | CCCATTGACGTCAATAATGACGTATGTTCCCATAGTAACGCCA |
| | | ATAGGGACTTTCCATTGACGTCAATGGGTGGAGTATTTACGCT |
| | | AAACTGCCCACTTGGCAGTACATCAAGTGTATCATATGCCAAG |
| | | TACGCCCCCTATTGACGTCAATGACGGTAAATGGCCCGCCTGG |
| | | CATTATGCCCAGTACATGACCTTATGGGACTTTCCTACTTGGCA |
| | | GTACATCTACGTATTAGTCATCGCTATTACCATG |
| 26 | rtTA4 | ATGTCCCGCTTGGATAAGAGCAAGGTAATAAATAGCGCACTCG |
| | Advanced | AACTCCTCAACGGCGTGGGCATCGAAGGTCTGACTACTCGAAA |
| | nucleic acid | GCTCGCCCAGAAATTGGGTGTGGAGCAACCTACATTGTATTGG |
| | sequence | CATGTCAAGAACAAAAGAGCCCTGCTGGACGCTCTTCCTATTG |
| | | AAATGCTTGACAGGCATCACACTCATTCCTGCCCCCTTGAGGT |
| | | CGAGAGTTGGCAAGATTTTCTCCGAAACAATGCAAAGTCCTAC |
| | | CGCTGCGCACTTTTGTCCCATAGGGATGGAGCAAAAGTGCACC |
| | | TGGGAACCAGGCCAACAGAGAAACAATACGAGACTCTCGAGA |
| | | ACCAGTTGGCTTTCTTGTGCCAACAGGGGTTCTCACTTGAAAAT |
| | | GCCCTTTACGCACTGTCAGCCGTTGGACATTTTACCCTGGGGTG |
| | | CGTTCTTGAGGAGCAAGAACATCAGGTTGCTAAGGAGGAGCG |
| | | CGAGACTCCAACCACTGATTCTATGCCACCTTTGCTGAAACAG |
| | | GCCATTGAACTTTTCGATAGACAGGGTGCTGAACCTGCCTTTCT |
| | | CTTCGGGTTGGAGCTGATTATTTGTGGTCTCGAAAAACAGCTG |
| | | AAATGTGAAAGTGGTGGCCCTACTGACGCCCTCGATGATTTCG |
| | | ACCTGGATATGCTGCCAGCCGATGCACTTGATGATTTCGATTTG |
| | | GATATGCTTCCAGCCGACGCACTGGACGACTTCGATTTGGACA |
| | | TGCTTCCCGGTTAA |
| 27 | rtTA4 | MSRLDKSKVINSALELLNGVGIEGLTTRKLAQKLGVEQPTLYWH |
| | Advanced | VKNKRALLDALPIEMLDRHHTHSCPLEVESWQDFLRNNAKSYRC |
| | amino acid | ALLSHRDGAKVHLGTRPTEKQYETLENQLAFLCQQGFSLENALY |
| | sequence | ALSAVGHFTLGCVLEEQEHQVAKEERETPTTDSMPPLLKQAIELF |
| | | DRQGAEPAFLFGLELIICGLEKQLKCESGGPTDALDDFDLDMLPA |
| | | DALDDFDLDMLPADALDDFDLDMLPG |
| 28 | AAV2- | TCGCGCGTTTCGGTGATGACGGTGAAAACCTCTGACACATGCA |
| | CMV- | GCTCCCGGAGACTGTCACAGCTTGTCTGTAAGCGGATGCCGGG |
| | rtTA4V16 | AGCAGACAAGCCCGTCAGGGCGCGTCAGCGGGTGTTGGCGGG |
| | | TGTCGGGGCTGGCTTAACTATGCGGCATCAGAGCAGATTGTAC |
| | | TGAGAGTGCACCATATGCGGTGTGAAATACCGCACAGATGCGT |
| | | AAGGAGAAAATACCGCATCAGGCGCCATTCGCCATTCAGGCTG |
| | | CGCAACTGTTGGGAAGGGCGATCGGTGCGGGCCTCTTCGCTAT |
| | | TACGCCAGCTGGCGAAAGGGGGATGTGCTGCAAGGCGATTAA |
| | | GTTGGGTAACGCCAGGGTTTTCCCAGTCACGACGTTGTAAAAC |
| | | GACGGCCAGTGAATTGACGCGTATTGGGATACTATGGTTGCTT |
| | | TGACGTATGCGGTGTGAAATACCGCACAGATGCGTAAGGAGA |
| | | AAATACCGCATCAGGCGCCCCTGCAGGCAGCTGCGCGCTCGCT |
| | | CGCTCACTGAGGCCGCCCGGGCAAAGCCCGGGCGTCGGGCGA |
| | | CCTTTGGTCGCCCGGCCTCAGTGAGCGAGCGAGCGCGCAGAGA |
| | | GGGAGTGGCCAACTCCATCACTAGGGGTTCCTGCGGCCGCTCG |
| | | GTCCGCACGATCTCAATTCGGCCATTACGGCCGGATCCGGCTC |
| | | GAGGAGCTTGGCCCATTGCATACGTTGTATCCATATCATAATA |
| | | TGTACATTTATATTGGCTCATGTCCAACATTACCGCCATGTTGA |
| | | CATTGATTATTGACTAGTTATTAATAGTAATCAATTACGGGGTC |
| | | ATTAGTTCATAGCCCATATATGGAGTTCCGCGTTACATAACTTA |
| | | CGGTAAATGGCCCGCCTGGCTGACCGCCCAACGACCCCCGCCC |
| | | ATTGACGTCAATAATGACGTATGTTCCCATAGTAACGCCAATA |
| | | GGGACTTTCCATTGACGTCAATGGGTGGAGTATTTACGCTAAA |
| | | CTGCCCACTTGGCAGTACATCAAGTGTATCATATGCCAAGTAC |
| | | GCCCCCTATTGACGTCAATGACGGTAAATGGCCCGCCTGGCAT |
| | | TATGCCCAGTACATGACCTTATGGGACTTTCCTACTTGGCAGTA |
| | | CATCTACGTATTAGTCATCGCTATTACCATGGTGATGCGGTTTT |
| | | GGCAGTACATCAATGGGCGTGGATAGCGGTTTGACTCACGGGG |
| | | ATTTCCAAGTCTCCACCCCATTGACGTCAATGGGAGTTTGTTTT |
| | | GGCACCAAAATCAACGGGACTTTCCAAAATGTCGTAACAACTC |
| | | CGCCCCATTGACGCAAATGGGCGGTAGGCGTGTACGGTGGGA |
| | | GGTCTATATAAGCAGAGCTCGTTTAGTGAACCGTCAGATCGCC |
| | | TGGAGACGCCATCCACGCTGTTTTGACCTCCATAGAAGACACC |
| | | GGGACCGATCCAGCCTCCGCGGCCCCGAATTCACCATGTCCCG |
| | | CTTGGATAAGAGCAAGGTAATAAATAGCGCACTCGAACTCCTC |
| | | AACGGCGTGGGCATCGAAGGTCTGACTACTCGAAAGCTCGCCC |
| | | AGAAATTGGGTGTGGAGCAACCTACATTGTATTGGCATGTCAA |
| | | GAACAAAAGAGCCCTGCTGGACGCTCTTCCTATTGAAATGCTT |
| | | GACAGGCATCACACTCATTCCTGCCCCCTTGAGGTCGAGAGTT |
| | | GGCAAGATTTTCTCCGAAACAATGCAAAGTCCTACCGCTGCGC |
| | | ACTTTTGTCCCATAGGGATGGAGCAAAAGTGCACCTGGGAACC |
| | | AGGCCAACAGAGAAACAATACGAGACTCTCGAGAACCAGTTG |
| | | GCTTTCTTGTGCCAACAGGGGTTCTCACTTGAAAATGCCCTTTA |
| | | CGCACTGTCAGCCGTTGGACATTTTACCCTGGGGTGCGTTCTTG |
| | | AGGAGCAAGAACATCAGGTTGCTAAGGAGGAGCGCGAGACTC |
| | | CAACCACTGATTCTATGCCACCTTTGCTGAAACAGGCCATTGA |
| | | ACTTTTCGATAGACAGGGTGCTGAACCTGCCTTTCTCTTCGGGT |
| | | TGGAGCTGATTATTTGTGGTCTCGAAAAACAGCTGAAATGTGA |
| | | AAGTGGTGGCCCTACTGACGCCCTCGATGATTTCGACCTGGAT |
| | | ATGCTGCCAGCCGATGCACTTGATGATTTCGATTTGGATATGCT |
| | | TCCAGCCGACGCACTGGACGACTTCGATTTGGACATGCTTCCC |
| | | GGTTAACTAAGTAAGGATCATCTTAATTAAATCGATAAGGATC |
| | | TGGCCGCCTCGGCCTAATCAACCTCTGGATTACAAAATTTGTG |
| | | AAAGATTGACTGGTATTCTTAACTATGTTGCTCCTTTTACGCTA |
| | | TGTGGATACGCTGCTTTAATGCCTTTGTATCATGCTATTGCTTC |
| | | CCGTATGGCTTTCATTTTCTCCTCCTTGTATAAATCCTGGTTGCT |
| | | GTCTCTTTATGAGGAGTTGTGGCCCGTTGTCAGGCAACGTGGC |
| | | GTGGTGTGCACTGTGTTTGCTGACGCAACCCCCACTGGTTGGG |
| | | GCATTGCCACCACCTGTCAGCTCCTTTCCGGGACTTTCGCTTTC |
| | | CCCCTCCCTATTGCCACGGCGGAACTCATCGCCGCCTGCCTTGC |
| | | CCGCTGCTGGACAGGGGCTCGGCTGTTGGGCACTGACAATTCC |
| | | GTGGTGTTGTCGGGGAAATCATCGTCCTTTCCTTGGCTGCTCGC |
| | | CTGTGTTGCCACCTGGATTCTGCGCGGGACGTCCTTCTGCTACG |
| | | TCCCTTCGGCCCTCAATCCAGCGGACCTTCCTTCCCGCGGCCTG |
| | | CTGCCGGCTCTGCGGCCTCTTCCGCGTCTTCGCCTTCGCCCTCA |
| | | GACGAGTCGGATCTCCCTTTGGGCCGCCTCCCCGCCAGACATG |
| | | ATAAGATACATTGATGAGTTTGGACAAACCACAACTAGAATGC |
| | | AGTGAAAAAAATGCTTTATTTGTGAAATTTGTGATGCTATTGCT |
| | | TTATTTGTAACCATTATAAGCTGCAATAAACAAGTTAACAACA |
| | | ACAATTGCATTCATTTTATGTTTCAGGTTCAGGGGGAGATGTG |
| | | GGAGGTTTTTTAAAGCAAGTAAAACCTCTACAAATGTGGTAAC |
| | | TAGCGCGTGCGGCCGCAGGAACCCCTAGTGATGGAGTTGGCCA |
| | | CTCCCTCTCTGCGCGCTCGCTCGCTCACTGAGGCCGGGCGACC |
| | | AAAGGTCGCCCGACGCCCGGGCTTTGCCCGGGCGGCCTCAGTG |
| | | AGCGAGCGAGCGCGCAGCTGCCTGCAGGATCCCAATGGCGCG |
| | | CCGAGCTTGGCTCGAGCATGGTCATAGCTGTTTCCTGTGTGAA |
| | | ATTGTTATCCGCTCACAATTCCACACAACATACGAGCCGGAAG |
| | | CATAAAGTGTAAAGCCTGGGGTGCCTAATGAGTGAGCTAACTC |
| | | ACATTAATTGCGTTGCGCTCACTGCCCGCTTTCCAGTCGGGAA |
| | | ACCTGTCGTGCCAGCTGCATTAATGAATCGGCCAACGCGCGGG |
| | | GAGAGGCGGTTTGCGTATTGGGCGCTGTTCCGCTTCCTCGCTCA |
| | | CTGACTCGCTGCGCTCGGTCGTTCGGCTGCGGCGAGCGGTATC |
| | | AGCTCACTCAAAGGCGGTAATACGGTTATCCACAGAATCAGGG |
| | | GATAACGCAGGAAAGAACATGTGAGCAAAAGGCCAGCAAAAG |
| | | GCCAGGAACCGTAAAAAGGCCGCGTTGCTGGCGTTTTTCCATA |
| | | GGCTCCGCCCCCCTGACGAGCATCACAAAAATCGACGCTCAAG |
| | | TCAGAGGTGGCGAAACCCGACAGGACTATAAAGATACCAGGC |
| | | GTTTCCCCCTGGAAGCTCCCTCGTGCGCTCTCCTGTTCCGACCC |
| | | TGCCGCTTACCGGATACCTGTCCGCCTTTCTCCCTTCGGGAAGC |
| | | GTGGCGCTTTCTCATAGCTCACGCTGTAGGTATCTCAGTTCGGT |
| | | GTAGGTCGTTCGCTCCAAGCTGGGCTGTGTGCACGAACCCCCC |
| | | GTTCAGCCCGACCGCTGCGCCTTATCCGGTAACTATCGTCTTGA |
| | | GTCCAACCCGGTAAGACACGACTTATCGCCACTGGCAGCAGCC |
| | | ACTGGTAACAGGATTAGCAGAGCGAGGTATGTAGGCGGTGCT |
| | | ACAGAGTTCTTGAAGTGGTGGCCTAACTACGGCTACACTAGAA |
| | | GAACAGTATTTGGTATCTGCGCTCTGCTGAAGCCAGTTACCTTC |
| | | GGAAAAAGAGTTGGTAGCTCTTGATCCGGCAAACAAACCACC |
| | | GCTGGTAGCGGTGGTTTTTTTGTTTGCAAGCAGCAGATTACGC |
| | | GCAGAAAAAAAGGATCTCAAGAAGATCCTTTGATCTTTTCTAC |
| | | GGGGTCTGACGCTCAGTGGAACGAAAACTCACGTTAAGGGATT |
| | | TTGGTCATGAGATTATCAAAAAGGATCTTCACCTAGATCCTTTT |
| | | AAATTAAAAATGAAGTTTTAAATCAATCTAAAGTATATATGAG |
| | | TAAACTTGGTCTGACAGTTAGAAAAACTCATCGAGCATCAAAT |
| | | GAAACTGCAATTTATTCATATCAGGATTATCAATACCATATTTT |
| | | TGAAAAAGCCGTTTCTGTAATGAAGGAGAAAACTCACCGAGG |
| | | CAGTTCCATAGGATGGCAAGATCCTGGTATCGGTCTGCGATTC |
| | | CGACTCGTCCAACATCAATACAACCTATTAATTTCCCCTCGTCA |
| | | AAAATAAGGTTATCAAGTGAGAAATCACCATGAGTGACGACT |
| | | GAATCCGGTGAGAATGGCAAAAGTTTATGCATTTCTTTCCAGA |
| | | CTTGTTCAACAGGCCAGCCATTACGCTCGTCATCAAAATCACT |
| | | CGCATCAACCAAACCGTTATTCATTCGTGATTGCGCCTGAGCG |
| | | AAACGAAATACGCGATCGCTGTTAAAAGGACAATTACAAACA |
| | | GGAATCGAATGCAACCGGCGCAGGAACACTGCCAGCGCATCA |
| | | ACAATATTTTCACCTGAATCAGGATATTCTTCTAATACCTGGAA |
| | | TGCTGTTTTCCCAGGGATCGCAGTGGTGAGTAACCATGCATCA |
| | | TCAGGAGTACGGATAAAATGCTTGATGGTCGGAAGAGGCATA |
| | | AATTCCGTCAGCCAGTTTAGTCTGACCATCTCATCTGTAACATC |
| | | ATTGGCAACGCTACCTTTGCCATGTTTCAGAAACAACTCTGGC |
| | | GCATCGGGCTTCCCATACAATCGATAGATTGTCGCACCTGATT |
| | | GCCCGACATTATCGCGAGCCCATTTATACCCATATAAATCAGC |
| | | ATCCATGTTGGAATTTAATCGCGGCCTAGAGCAAGACGTTTCC |
| | | CGTTGAATATGGCTCATACTCTTCCTTTTTCAATATTATTGAAG |
| | | CATTTATCAGGGTTATTGTCTCATGAGCGGATACATATTTGAAT |
| | | GTATTTAGAAAAATAAACAAATAGGGGTTCCGCGCACATTTCC |
| | | CCGAAAAGTGCCACCTGACGTCTAAGAAACCATTATTATCATG |
| | | ACATTAACCTATAAAAATAGGCGTATCACGAGGCCCTTTTGTC |
| 29 | ITR- | CCTGCAGGCAGCTGCGCGCTCGCTCGCTCACTGAGGCCGCCCG |
| | forward | GGCAAAGCCCGGGCGTCGGGCGACCTTTGGTCGCCCGGCCTCA |
| | | GTGAGCGAGCGAGCGCGCAGAGAGGGAGTGGCCAACTCCATC |
| | | ACTAGGGGTTCCT |
| 34 | ITR- | AGGAACCCCTAGTGATGGAGTTGGCCACTCCCTCTCTGCGCGC |
| | reverse | TCGCTCGCTCACTGAGGCCGGGCGACCAAAGGTCGCCCGACGC |
| | | CCGGGCTTTGCCCGGGCGGCCTCAGTGAGCGAGCGAGCGCGCA |
| | | GCTGCCTGCAGG |
| 30 | SV40p | TAAGATACATTGATGAGTTTGGACAAACCACAACTAGAATGCA |
| | | GTGAAAAAAATGCTTTATTTGTGAAATTTGTGATGCTATTGCTT |
| | | TATTTGTAACCATTATAAGCTGCAATAAACAAGTT |
| 31 | WPRE | AATCAACCTCTGGATTACAAAATTTGTGAAAGATTGACTGGTA |
| | | TTCTTAACTATGTTGCTCCTTTTACGCTATGTGGATACGCTGCT |
| | | TTAATGCCTTTGTATCATGCTATTGCTTCCCGTATGGCTTTCATT |
| | | TTCTCCTCCTTGTATAAATCCTGGTTGCTGTCTCTTTATGAGGA |
| | | GTTGTGGCCCGTTGTCAGGCAACGTGGCGTGGTGTGCACTGTG |
| | | TTTGCTGACGCAACCCCCACTGGTTGGGGCATTGCCACCACCT |
| | | GTCAGCTCCTTTCCGGGACTTTCGCTTTCCCCCTCCCTATTGCC |
| | | ACGGCGGAACTCATCGCCGCCTGCCTTGCCCGCTGCTGGACAG |
| | | GGGCTCGGCTGTTGGGCACTGACAATTCCGTGGTGTTGTCGGG |
| | | GAAATCATCGTCCTTTCCTTGGCTGCTCGCCTGTGTTGCCACCT |
| | | GGATTCTGCGCGGGACGTCCTTCTGCTACGTCCCTTCGGCCCTC |
| | | AATCCAGCGGACCTTCCTTCCCGCGGCCTGCTGCCGGCTCTGC |
| | | GGCCTCTTCCGCGTCTTCGCCTTCGCCCTCAGACGAGTCGGATC |
| | | TCCCTTTGGGCCGCCTCCCCGC |
| 37 | Full CMV- | CCTGCAGGCAGCTGCGCGCTCGCTCGCTCACTGAGGCCGCCCG |
| | rtTA4V16 | GGCAAAGCCCGGGCGTCGGGCGACCTTTGGTCGCCCGGCCTCA |
| | sequence | GTGAGCGAGCGAGCGCGCAGAGAGGGAGTGGCCAACTCCATC |
| | (including | ACTAGGGGTTCCTGCGGCCGCTCGGTCCGCACGATCTCAATTC |
| | ITRs) | GGCCATTACGGCCGGATCCGGCTCGAGGAGCTTGGCCCATTGC |
| | | ATACGTTGTATCCATATCATAATATGTACATTTATATTGGCTCA |
| | | TGTCCAACATTACCGCCATGTTGACATTGATTATTGACTAGTTA |
| | | TTAATAGTAATCAATTACGGGGTCATTAGTTCATAGCCCATAT |
| | | ATGGAGTTCCGCGTTACATAACTTACGGTAAATGGCCCGCCTG |
| | | GCTGACCGCCCAACGACCCCCGCCCATTGACGTCAATAATGAC |
| | | GTATGTTCCCATAGTAACGCCAATAGGGACTTTCCATTGACGT |
| | | CAATGGGTGGAGTATTTACGCTAAACTGCCCACTTGGCAGTAC |
| | | ATCAAGTGTATCATATGCCAAGTACGCCCCCTATTGACGTCAA |
| | | TGACGGTAAATGGCCCGCCTGGCATTATGCCCAGTACATGACC |
| | | TTATGGGACTTTCCTACTTGGCAGTACATCTACGTATTAGTCAT |
| | | CGCTATTACCATGGTGATGCGGTTTTGGCAGTACATCAATGGG |
| | | CGTGGATAGCGGTTTGACTCACGGGGATTTCCAAGTCTCCACC |
| | | CCATTGACGTCAATGGGAGTTTGTTTTGGCACCAAAATCAACG |
| | | GGACTTTCCAAAATGTCGTAACAACTCCGCCCCATTGACGCAA |
| | | ATGGGCGGTAGGCGTGTACGGTGGGAGGTCTATATAAGCAGA |
| | | GCTCGTTTAGTGAACCGTCAGATCGCCTGGAGACGCCATCCAC |
| | | GCTGTTTTGACCTCCATAGAAGACACCGGGACCGATCCAGCCT |
| | | CCGCGGCCCCGAATTCACCATGTCCCGCTTGGATAAGAGCAAG |
| | | GTAATAAATAGCGCACTCGAACTCCTCAACGGCGTGGGCATCG |
| | | AAGGTCTGACTACTCGAAAGCTCGCCCAGAAATTGGGTGTGGA |
| | | GCAACCTACATTGTATTGGCATGTCAAGAACAAAAGAGCCCTG |
| | | CTGGACGCTCTTCCTATTGAAATGCTTGACAGGCATCACACTC |
| | | ATTCCTGCCCCCTTGAGGTCGAGAGTTGGCAAGATTTTCTCCGA |
| | | AACAATGCAAAGTCCTACCGCTGCGCACTTTTGTCCCATAGGG |
| | | ATGGAGCAAAAGTGCACCTGGGAACCAGGCCAACAGAGAAAC |
| | | AATACGAGACTCTCGAGAACCAGTTGGCTTTCTTGTGCCAACA |
| | | GGGGTTCTCACTTGAAAATGCCCTTTACGCACTGTCAGCCGTTG |
| | | GACATTTTACCCTGGGGTGCGTTCTTGAGGAGCAAGAACATCA |
| | | GGTTGCTAAGGAGGAGCGCGAGACTCCAACCACTGATTCTATG |
| | | CCACCTTTGCTGAAACAGGCCATTGAACTTTTCGATAGACAGG |
| | | GTGCTGAACCTGCCTTTCTCTTCGGGTTGGAGCTGATTATTTGT |
| | | GGTCTCGAAAAACAGCTGAAATGTGAAAGTGGTGGCCCTACTG |
| | | ACGCCCTCGATGATTTCGACCTGGATATGCTGCCAGCCGATGC |
| | | ACTTGATGATTTCGATTTGGATATGCTTCCAGCCGACGCACTGG |
| | | ACGACTTCGATTTGGACATGCTTCCCGGTTAACTAAGTAAGGA |
| | | TCATCTTAATTAAATCGATAAGGATCTGGCCGCCTCGGCCTAA |
| | | TCAACCTCTGGATTACAAAATTTGTGAAAGATTGACTGGTATT |
| | | CTTAACTATGTTGCTCCTTTTACGCTATGTGGATACGCTGCTTT |
| | | AATGCCTTTGTATCATGCTATTGCTTCCCGTATGGCTTTCATTTT |
| | | CTCCTCCTTGTATAAATCCTGGTTGCTGTCTCTTTATGAGGAGT |
| | | TGTGGCCCGTTGTCAGGCAACGTGGCGTGGTGTGCACTGTGTT |
| | | TGCTGACGCAACCCCCACTGGTTGGGGCATTGCCACCACCTGT |
| | | CAGCTCCTTTCCGGGACTTTCGCTTTCCCCCTCCCTATTGCCAC |
| | | GGCGGAACTCATCGCCGCCTGCCTTGCCCGCTGCTGGACAGGG |
| | | GCTCGGCTGTTGGGCACTGACAATTCCGTGGTGTTGTCGGGGA |
| | | AATCATCGTCCTTTCCTTGGCTGCTCGCCTGTGTTGCCACCTGG |
| | | ATTCTGCGCGGGACGTCCTTCTGCTACGTCCCTTCGGCCCTCAA |
| | | TCCAGCGGACCTTCCTTCCCGCGGCCTGCTGCCGGCTCTGCGGC |
| | | CTCTTCCGCGTCTTCGCCTTCGCCCTCAGACGAGTCGGATCTCC |
| | | CTTTGGGCCGCCTCCCCGCCAGACATGATAAGATACATTGATG |
| | | AGTTTGGACAAACCACAACTAGAATGCAGTGAAAAAAATGCTT |
| | | TATTTGTGAAATTTGTGATGCTATTGCTTTATTTGTAACCATTA |
| | | TAAGCTGCAATAAACAAGTTAACAACAACAATTGCATTCATTT |
| | | TATGTTTCAGGTTCAGGGGGAGATGTGGGAGGTTTTTTAAAGC |
| | | AAGTAAAACCTCTACAAATGTGGTAACTAGCGCGTGCGGCCGC |
| | | AGGAACCCCTAGTGATGGAGTTGGCCACTCCCTCTCTGCGCGC |
| | | TCGCTCGCTCACTGAGGCCGGGCGACCAAAGGTCGCCCGACGC |
| | | CCGGGCTTTGCCCGGGCGGCCTCAGTGAGCGAGCGAGCGCGCA |
| | | GCTGCCTGCAGGATCCCAATGGCGCGCCGAGCTTGGCTCGAGC |
| | | ATGGTCATAGCTGTTTCCTGTGTGAAATTGTTATCCGCTCACAA |
| | | TTCCACACAACATACGAGCCGGAAGCATAAAGTGTAAAGCCTG |
| | | GGGTGCCTAATGAGTGAGCTAACTCACATTAATTGCGTTGCGC |
| | | TCACTGCCCGCTTTCCAGTCGGGAAACCTGTCGTGCCAGCTGC |
| | | ATTAATGAATCGGCCAACGCGCGGGGAGAGGCGGTTTGCGTAT |
| | | TGGGCGCTGTTCCGCTTCCTCGCTCACTGACTCGCTGCGCTCGG |
| | | TCGTTCGGCTGCGGCGAGCGGTATCAGCTCACTCAAAGGCGGT |
| | | AATACGGTTATCCACAGAATCAGGGGATAACGCAGGAAAGAA |
| | | CATGTGAGCAAAAGGCCAGCAAAAGGCCAGGAACCGTAAAAA |
| | | GGCCGCGTTGCTGGCGTTTTTCCATAGGCTCCGCCCCCCTGACG |
| | | AGCATCACAAAAATCGACGCTCAAGTCAGAGGTGGCGAAACC |
| | | CGACAGGACTATAAAGATACCAGGCGTTTCCCCCTGGAAGCTC |
| | | CCTCGTGCGCTCTCCTGTTCCGACCCTGCCGCTTACCGGATACC |
| | | TGTCCGCCTTTCTCCCTTCGGGAAGCGTGGCGCTTTCTCATAGC |
| | | TCACGCTGTAGGTATCTCAGTTCGGTGTAGGTCGTTCGCTCCAA |
| | | GCTGGGCTGTGTGCACGAACCCCCCGTTCAGCCCGACCGCTGC |
| | | GCCTTATCCGGTAACTATCGTCTTGAGTCCAACCCGGTAAGAC |
| | | ACGACTTATCGCCACTGGCAGCAGCCACTGGTAACAGGATTAG |
| | | CAGAGCGAGGTATGTAGGCGGTGCTACAGAGTTCTTGAAGTGG |
| | | TGGCCTAACTACGGCTACACTAGAAGAACAGTATTTGGTATCT |
| | | GCGCTCTGCTGAAGCCAGTTACCTTCGGAAAAAGAGTTGGTAG |
| | | CTCTTGATCCGGCAAACAAACCACCGCTGGTAGCGGTGGTTTT |
| | | TTTGTTTGCAAGCAGCAGATTACGCGCAGAAAAAAAGGATCTC |
| | | AAGAAGATCCTTTGATCTTTTCTACGGGGTCTGACGCTCAGTG |
| | | GAACGAAAACTCACGTTAAGGGATTTTGGTCATGAGATTATCA |
| | | AAAAGGATCTTCACCTAGATCCTTTTAAATTAAAAATGAAGTT |
| | | TTAAATCAATCTAAAGTATATATGAGTAAACTTGGTCTGACAG |
| | | TTAGAAAAACTCATCGAGCATCAAATGAAACTGCAATTTATTC |
| | | ATATCAGGATTATCAATACCATATTTTTGAAAAAGCCGTTTCTG |
| | | TAATGAAGGAGAAAACTCACCGAGGCAGTTCCATAGGATGGC |
| | | AAGATCCTGGTATCGGTCTGCGATTCCGACTCGTCCAACATCA |
| | | ATACAACCTATTAATTTCCCCTCGTCAAAAATAAGGTTATCAA |
| | | GTGAGAAATCACCATGAGTGACGACTGAATCCGGTGAGAATG |
| | | GCAAAAGTTTATGCATTTCTTTCCAGACTTGTTCAACAGGCCAG |
| | | CCATTACGCTCGTCATCAAAATCACTCGCATCAACCAAACCGT |
| | | TATTCATTCGTGATTGCGCCTGAGCGAAACGAAATACGCGATC |
| | | GCTGTTAAAAGGACAATTACAAACAGGAATCGAATGCAACCG |
| | | GCGCAGGAACACTGCCAGCGCATCAACAATATTTTCACCTGAA |
| | | TCAGGATATTCTTCTAATACCTGGAATGCTGTTTTCCCAGGGAT |
| | | CGCAGTGGTGAGTAACCATGCATCATCAGGAGTACGGATAAA |
| | | ATGCTTGATGGTCGGAAGAGGCATAAATTCCGTCAGCCAGTTT |
| | | AGTCTGACCATCTCATCTGTAACATCATTGGCAACGCTACCTTT |
| | | GCCATGTTTCAGAAACAACTCTGGCGCATCGGGCTTCCCATAC |
| | | AATCGATAGATTGTCGCACCTGATTGCCCGACATTATCGCGAG |
| | | CCCATTTATACCCATATAAATCAGCATCCATGTTGGAATTTAAT |
| | | CGCGGCCTAGAGCAAGACGTTTCCCGTTGAATATGGCTCATAC |
| | | TCTTCCTTTTTCAATATTATTGAAGCATTTATCAGGGTTATTGTC |
| | | TCATGAGCGGATACATATTTGAATGTATTTAGAAAAATAAACA |
| | | AATAGGGGTTCCGCGCACATTTCCCCGAAAAGTGCCACCTGAC |
| | | GTCTAAGAAACCATTATTATCATGACATTAACCTATAAAAATA |
| | | GGCGTATCACGAGGCCCTTTTGTC |

1. A method for treating non-arteritic anterior ischemic optic neuropathy (NAION) in a subject in need thereof,
the method comprising intravitreally administering, to one or both eyes of a subject treated with a tetracycline-class antibiotic before, during and/or after the administering step, a pharmaceutically effective amount of a composition comprising:
a first adeno-associated virus (AAV) expression vector comprising a first polynucleotide encoding octamer-binding transcription factor 4 (OCT4), sex determining region Y)-box 2 (SOX2), and Kruppel-like factor 4 (KLF4), but not Myc proto-oncogene (c-Myc), operatively linked to a tetracycline response element (TRE) promoter and flanked by inverted terminal repeats (ITRs); and
a second adeno-associated virus (AAV) expression vector comprising a second polynucleotide encoding a reverse tetracycline transactivator, operatively linked to a promoter and flanked by inverted terminal repeats (ITRs); or
sequentially, in any order, administering pharmaceutically effective amounts of the first AAV expression vector and of the second AAV expression vector.

2. The method of claim 1, wherein the composition does not reprogram a cell, tissue, or organ to a pluripotent state in the subject.

3. (canceled)

4. (canceled)

5. (canceled)

6. (canceled)

7. (canceled)

8. (canceled)

9. The method of claim 1, further comprising administering to the subject the tetracycline-class antibiotic before the adeno-associated virus (AAV) expression vectors.

10. (canceled)

11. The method of claim 1, wherein the tetracycline class antibiotic is doxycycline.

12. (canceled)

13. (canceled)

14. The method of claim 9, further comprising administering to the subject the tetracycline-class antibiotic after the adeno-associated virus (AAV) expression vectors.

15. (canceled)

16. The method of claim 1, wherein the first polynucleotide encodes a self-cleaving peptide between the OCT4, the SOX2, and/or the KLF4.

17. The method of claim 16, wherein the self-cleaving peptide is a 2A peptide.

18. (canceled)

19. (canceled)

20. The method of claim 19, wherein the AAV vector is serotype-2 (AAV2).

21. The method of claim 1, wherein the first polynucleotide comprises nucleic acid elements in the following order:
a. a first inverted terminal repeat sequence (ITR) sequence;
b. a TRE3G promoter sequence;
c. an OCT4 sequence;
d. a P2A cleavage sequence;
e. a SOX2 sequence;
f. a T2A cleavage sequence;
g. a KLF4 sequence;
h. an SV-40-derived terminator sequence; and
i. a second inverted terminal repeat (ITR) sequence.

22. The method of claim 1, wherein
i) OCT4 comprises an amino acid sequence having at least 90% identity to SEQ ID NO: 2;
ii) SOX2 comprises an amino acid sequence having at least 90% identity to SEQ ID NO: 4; and/or
iii) KLF4 comprises an amino acid sequence having at least 90% identity to SEQ ID NO: 6.

23. The method of claim 1, wherein
i) the polynucleotide comprises a nucleic acid sequence having at least 75%90% identity to SEQ ID NO: 1;
ii) the polynucleotide comprises a nucleic acid sequence having at least 75%90% identity to SEQ ID NO: 3; and/or
iii) the polynucleotide comprises a nucleic acid sequence having at least 75%90% identity to SEQ ID NO: 5.

24. (canceled)

25. (canceled)

26. The method of claim 1, wherein administering the composition improves retinal ganglion cell (RGC) function.

27. The method of claim 26, wherein RGC function is measured by electroretinogram (pERG).

28. The method of claim 1, wherein
i) OCT4 comprises an amino acid sequence having at least 95% identity to SEQ ID NO: 2;
ii) SOX2 comprises an amino acid sequence having at least 95% identity to SEQ ID NO: 4; and/or
iii) KLF4 comprises an amino acid sequence having at least 95% identity to SEQ ID NO: 6.

29. The method of claim 1, wherein
i) OCT4 comprises the amino acid sequence of SEQ ID NO: 2;
ii) SOX2 comprises the amino acid sequence of SEQ ID NO: 4; and/or
iii) KLF4 comprises the amino acid sequence of SEQ ID NO: 6.

30. (canceled)

31. A method for treating non-arteritic anterior ischemic optic neuropathy (NAION) in a subject in need thereof,
the method comprising intravitreally administering, to one or both eyes of a subject treated with a doxycycline before, during and/or after the administering step, a pharmaceutically effective amount of a composition comprising:
a first adeno-associated virus of serotype 2 (AAV2) expression vector comprising a first polynucleotide encoding only three transcription factors, operatively linked to a tetracycline response element (TRE) promoter and flanked by inverted terminal repeats (ITRs); and
a second adeno-associated virus of serotype 2 (AAV2) expression vector comprising a second polynucleotide encoding only a reverse tetracycline transactivator, operatively linked to a promoter and flanked by inverted terminal repeats (ITRs),
wherein the only three transcription factors are octamer-binding transcription factor 4 (OCT4), sex determining region Y)-box 2 (SOX2), and Kruppel-like factor 4 (KLF4); or
sequentially, in any order, administering pharmaceutically effective amounts of the first AAV expression vector and of the second AAV expression vector.

32. The method of claim 31, wherein the composition does not reprogram a cell, tissue, or organ to a pluripotent state in the subject.

33. The method of claim 31, wherein the composition does not induce c-Myc expression in the subject.

34. The method of claim 31, further comprising administering to the subject the doxycycline before the adeno-associated virus (AAV) expression.

35. The method of claim 31, further comprising administering to the subject the doxycycline after the adeno-associated virus (AAV) expression vectors.

36. The method of claim 31, further comprising administering to the subject the doxycycline before and after the adeno-associated virus (AAV) expression vectors.

37. The method of claim 31, wherein the first polynucleotide encodes OCT4, the SOX2, and the KLF4 linked by two self-cleaving peptide.

38. The method of claim 37, wherein the two self-cleaving peptide are each a 2A peptide.

39. The method of claim 31, wherein the first polynucleotide comprises nucleic acid elements in the following order:
a. a first inverted terminal repeat sequence (ITR) sequence;
b. a TRE3G promoter sequence;
c. an OCT4 sequence;
d. a P2A cleavage sequence;
e. a SOX2 sequence;
f. a T2A cleavage sequence;
g. a KLF4 sequence;
h. an SV-40-derived terminator sequence; and
i. a second inverted terminal repeat (ITR) sequence.

40. The method of claim 39, wherein
i) OCT4 comprises an amino acid sequence having at least 90% identity to SEQ ID NO: 2;
ii) SOX2 comprises an amino acid sequence having at least 90% identity to SEQ ID NO: 4; and
iii) KLF4 comprises an amino acid sequence having at least 90% identity to SEQ ID NO: 6.

41. The method of claim 1, wherein
i) OCT4 comprises an amino acid sequence having at least 95% identity to SEQ ID NO: 2;
ii) SOX2 comprises an amino acid sequence having at least 95% identity to SEQ ID NO: 4; and/or
iii) KLF4 comprises an amino acid sequence having at least 95% identity to SEQ ID NO: 6.

42. A method for treating non-arteritic anterior ischemic optic neuropathy (NAION) in a subject in need thereof,
the method comprising intravitreally administering, to one or both eyes of a subject treated with a doxycycline before, during and/or after the administering step, a pharmaceutically effective amount of a composition comprising:
a first adeno-associated virus of serotype 2 (AAV2) expression vector comprising a first polynucleotide encoding only three transcription factors, operatively linked to a TRE3G promoter, and flanked by inverted terminal repeats (ITRs); and
a second adeno-associated virus of serotype 2 (AAV2) expression vector comprising a second polynucleotide encoding only a rtTA3VP16, operatively limited to a CMV promoter, and flanked by inverted terminal repeats (ITRs),
wherein the only three transcription factors are octamer-binding transcription factor 4 (OCT4), sex determining region Y)-box 2 (SOX2), and Kruppel-like factor 4 (KLF4); or
sequentially, in any order, administering pharmaceutically effective amounts of the first AAV expression vector and of the second AAV expression vector.

43. The method of claim 1, wherein administering the composition restores visual function in the subject.

44. The method of claim 42, wherein administering the composition improves retinal ganglion cell (RGC) function.

45. The method of claim 46, wherein retinal ganglion cell (RGC) function is measured by electroretinogram (pERG).


SEQUENCE LISTING (37 sequences)

SEQ ID NO: 1 - DNA, 1080 bp, Homo sapiens
  source 1..1080: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaac

SEQ ID NO: 2 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - DNA, 951 bp, Homo sapiens
  source 1..951: mol_type = other DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatg

SEQ ID NO: 4 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - DNA, 1410 bp, Homo sapiens
  source 1..1410: mol_type = other DNA
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttt

SEQ ID NO: 6 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 7 - DNA, 376 bp, synthetic construct
  source 1..376: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 8 - DNA, 57 bp, Teschovirus A
  source 1..57: mol_type = other DNA
gccacgaacttctctctgttaaagcaagcaggagatgttgaagaaaaccccgggcct

SEQ ID NO: 9 - protein, 19 aa, Teschovirus A
ATNFSLLKQAGDVEENPGP

SEQ ID NO: 10 - DNA, 54 bp, Thosea asigna virus
  source 1..54: mol_type = other DNA
gagggcaggggaagtcttctaacatgcggggacgtggaggaaaatcccggccca

SEQ ID NO: 11 - protein, 18 aa, Thosea asigna virus
EGRGSLLTCGDVEENPGP

SEQ ID NO: 12 - DNA, 135 bp, synthetic construct
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 13 - DNA, 4933 bp, synthetic construct
  source 1..4933: mol_type = other DNA
ctatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcatcccaatggcgcgccgagcttggctcgagcatggtcat

SEQ ID NO: 14 - DNA, 3594 bp, synthetic construct
  source 1..3594: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttt

SEQ ID NO: 15 - DNA, 7250 bp, synthetic construct
  source 1..7250: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 16 - DNA, 141 bp, synthetic construct
  source 1..141: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 17 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 18 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 19 - DNA, 747 bp, synthetic construct
  source 1..747: mol_type = other DNA
atgtctagactggacaagagcaaaatcataaacagcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacagctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaagcaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 20 - protein, 248 aa, synthetic construct
MSRLDKSKIINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 21 - DNA, 5435 bp, synthetic construct
  source 1..5435: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaaatcataaacagcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacagctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaagcaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 22 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 23 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 24 - DNA, 204 bp, synthetic construct; identical to SEQ ID NO: 17
  source 1..204: mol_type = other DNA

SEQ ID NO: 25 - DNA, 380 bp, synthetic construct; identical to SEQ ID NO: 18
  source 1..380: mol_type = other DNA

SEQ ID NO: 26 - DNA, 747 bp, synthetic construct
  source 1..747: mol_type = other DNA
atgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaa

SEQ ID NO: 27 - protein, 248 aa, synthetic construct
MSRLDKSKVINSALELLNGVGIEGLTTRKLAQKLGVEQPTLYWHVKNKRALLDALPIEMLDRHHTHSCPLEVESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 28 - DNA, 5435 bp, synthetic construct
  source 1..5435: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 29 - DNA, 141 bp, synthetic construct; identical to SEQ ID NO: 22
  source 1..141: mol_type = other DNA

SEQ ID NO: 30 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 31 - DNA, 589 bp, synthetic construct; identical to SEQ ID NO: 23
  source 1..589: mol_type = other DNA

SEQ ID NO: 32 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 141 bp, synthetic construct; identical to SEQ ID NO: 32
  source 1..141: mol_type = other DNA

SEQ ID NO: 34 - DNA, 141 bp, synthetic construct; identical to SEQ ID NO: 32
  source 1..141: mol_type = other DNA

SEQ ID NO: 35 - DNA, 4460 bp, synthetic construct
  source 1..4460: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaaccccta

SEQ ID NO: 36 - DNA, 2735 bp, synthetic construct
  source 1..2735: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaaatcataaacagcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacagctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaagcaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 37 - DNA, 4946 bp, synthetic construct
  source 1..4946: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc